(19) United States
(12) Patent Application Publication
(10) Pub. No.: US 2006/0117401 A1
(43) Pub. Date: Jun. 1, 2006

(54) Method for producing aminoacids

(76) Inventors: Oliver Schmitz, Dallgow-Doberitz (DE); Piotr Puzio, Berlin (DE); Astrid Blau, Stahnsdorf (DE); Ralf Looser, Berlin (DE); Birgit Wendel, Berlin (DE); Beate Kamlage, Berlin (DE); Gunnar Plesch, Potsdam (DE)

Correspondence Address: CONNOLLY BOVE LODGE & HUTZ, LLP, P O BOX 2207, WILMINGTON, DE, 19899, US

(21) Appl. No.: 10/539,954

(22) PCT Filed: Dec. 19, 2003
(86) PCT No.: PCT/EP03/14649

§ 371 Date: Jun. 17, 2005

(30) Foreign Application Priority Data
Dec. 20, 2002 (DE) 102 61 188.2

(51) Int. Cl. A01H 1/00; C07H 21/04; C12P 13/04; C12N 15/87; C12N 1/21; C12N 1/18; C12N 15/82; C12N 15/74

(52) U.S. Cl. 800/278; 435/106; 435/252.3; 536/23.2; 435/468; 435/471; 435/254.21; 435/483

(57) ABSTRACT

The invention relates to a method for producing aminoacids in transgenic organisms. The inventive method consists of the following steps: a) introduction of nucleic acids sequence which codes threonine decomposing protein or lysine decomposing protein or codes threonine decomposing protein and lysine decomposing protein, b) introduction of nucleic acids sequence which improves the decomposition of threonine or lysine or the decomposition of threonine and lysine in the transgenic organisms; c) expression of (a) or (b) nucleic acids sequence in a transgenic organism. In a very useful manner, the nucleic acids sequence is introduced in the step a) of the method, said sequence being selected from: i) the nucleic acids sequence with the sequence present in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 and/or SEQ ID NO:25; ii) the nucleic acids sequence which is preserved as a result of a degenerate genetic code by re-recording aminoacids sequence present in SEQ ID NO: 2, SEQ ID NO: 12, SEQ ID NO: 14, SEQ ID NO: 16, SEQ ID NO:18, SEQ ID NO:20, SEQ ID NO:22, SEQ ID NO:24 and/or 26; and iii) a derivative of the nucleic add sequence present in SEQ ID NO: 1, SEQ ID NO:11, SEQ ID NO: 13, SEQ ID NO: 15, SEQ ID NO: 17, SEQ ID NO: 19, SEQ ID NO: 21, SEQ ID NO: 23 and/or SEQ ID NO:25 which codes polypeptides with the nucleic acids sequence present in SEQ ID NO: 2, SEQ ID NO: 12, SEQ ID NO: 14, SEQ ID NO: 16, SEQ ID NO: 18, SEQ ID NO:20, SEQ ID NO:22, SEQ ID NO:24 and/or 26 and which comprises at least 50% of homology in terms of aminoacids without reducing the biological activity of polypeptides.

The present invention relates to a process for preparing amino acids in transgenic organisms.

The invention further relates to nucleic acid constructs, vectors and transgenic organisms, and to the use thereof.

Amino acids form the basic structural unit of all proteins and are thus essential for normal cell functions. The term “amino acid” is known in the art. The proteogenic amino acids, of which there are 20 types, serve as structural units for proteins in which they are linked together via peptide bonds, whereas the non-proteogenic amino acids (of which hundreds are known) usually do not occur in proteins [see Ullmann's Encyclopedia of Industrial Chemistry, Vol. A2, pages 57-97 VCH: Weinheim (1985)]. The amino acids can exist in the D or L configuration, although L-amino acids are usually the only type found in naturally occurring proteins. Biosynthetic and degradation pathways of each of the 20 proteogenic amino acids are well characterized both in prokaryotic and eukaryotic cells (see, for example, Stryer, L. Biochemistry, 3rd edition, pages 578-590 (1988)). The “essential” amino acids (histidine, isoleucine, leucine, lysine, methionine, phenylalanine, threonine, tryptophan and valine), so called because they must be obtained through the diet because of the complexity of their biosynthesis, are converted by simple biosynthetic pathways into the other 11 “nonessential” amino acids (alanine, arginine, asparagine, aspartic acid, cysteine, glutamic acid, glutamine, glycine, proline, serine and tyrosine). Higher animals have the ability to synthesize some of these amino acids, but the essential amino acids must be obtained from food for normal protein synthesis to take place.

Amino acids are used in many branches of industry, including the human and animal food, cosmetics, pharmaceutical and chemical industries. Thus, L-glutamic acid is used for example in infusion solutions. Amino acids such as D,L-methionine, L-lysine or L-threonine are used in the animal food industry. Particularly important for the diet of humans and many useful animals are the essential amino acids valine, leucine, isoleucine, lysine, threonine, methionine, tyrosine, phenylalanine and tryptophan. Thus, for example, lysine is an important amino acid not only for the human diet but also for monogastric animals such as poultry and pigs. L-Lysine is the limiting amino acid in plants such as corn or wheat, which is to say that in order to enable optimal utilization of such plant food it is sensible to supplement the human or animal food with L-lysine. Glutamate is most frequently used as flavor additive (monosodium glutamate, MSG) and is used widely in the food industry, as are aspartate, phenylalanine, glycine and cysteine. Glycine, L-methionine and tryptophan are all used in the pharmaceutical industry. Glutamine, valine, leucine, isoleucine, histidine, arginine, proline, serine and alanine are used in the pharmaceutical industry and the cosmetics industry. Threonine, tryptophan and D/L-methionine are widely used animal food additives [Leuchtenberger, W. (1996) Amino acids—technical production and use, pages 466-502 in Rehm et al., (editors) Biotechnology Vol. 6, Chapter 14a, VCH: Weinheim]. In addition, amino acids are suitable for the chemical industry as precursors for synthesizing synthetic amino acids and proteins such as N-acetylcysteine, S-carboxymethyl-L-cysteine, (S)-5-hydroxytryptophan and other substances described in Ullmann's Encyclopedia of Industrial Chemistry, Vol. A2, pages 57-97. VCH, Weinheim, 1985.

The annual production of amino acids currently amounts to over 1 million t/a with a market value of more than 2 billion US$. They are at present produced by four competing processes:
1. extraction from protein hydrolysates, for example of L-cystine, L-leucine or L-tyrosine,
2. chemical synthesis, for example of D,L-methionine,
3. conversion of chemical precursors in an enzyme or cell reactor, for example L-phenylalanine and
4. fermentative preparation by large-scale culturing of bacteria developed in order to produce and separate large amounts of the particular desired molecule. An organism particularly suitable for this purpose is Corynebacterium glutamicum, which is used for example to prepare L-lysine or L-glutamic acid. Further examples of amino acids prepared by fermentation are L-threonine, L-tryptophan, L-aspartic acid and L-phenylalanine.

The biosynthesis of natural amino acids in organisms able to produce them, for example bacteria, has been well characterized [for a review of bacterial amino acid biosynthesis and its regulation, see Umbarger, H. E. (1978) Ann. Rev. Biochem. 47: 533-606]. Glutamic acid is synthesized by reductive amination of α-ketoglutarate, an intermediate in the citric acid cycle. Glutamine, proline and arginine are each produced successively from glutamate. The biosynthesis of serine takes place in a three-step process starting with 3-phosphoglycerate (a glycolysis intermediate) and resulting, after oxidation, transamination and hydrolysis steps, in this amino acid. Cysteine and glycine are each produced from serine, the former by condensation of homocysteine with serine, and the latter by transfer of the side-chain β carbon atom to tetrahydrofolate in a reaction catalyzed by serine transhydroxymethylase. Phenylalanine and tyrosine are synthesized from the precursors of the glycolysis pathway and pentose phosphate pathway, erythrose 4-phosphate and phosphoenolpyruvate in a 9-step biosynthetic pathway differing only in the last two steps after the synthesis of prephenate. Tryptophan is likewise produced from these two starting molecules but it is synthesized in an 11-step pathway. Tyrosine can also be produced from phenylalanine in a reaction catalyzed by phenylalanine hydroxylase. Alanine, valine and leucine are each biosynthetic products of pyruvate, the final product of glycolysis. Aspartic acid is formed from oxalacetate, an intermediate in the citric acid cycle. Asparagine, methionine, threonine and lysine are each produced by conversion of aspartic acid. Isoleucine is formed from threonine. Histidine is formed from 5-phosphoribosyl 1-pyrophosphate, an activated sugar, in a complex 9-step pathway.

The preparation of amino acids by fermentation of strains of coryneform bacteria, especially Corynebacterium glutamicum, is known. Because of the great importance, there is continuous work on improving the existing preparation processes. Process improvements may relate to measures of fermentation technique, such as, for example, stirring and oxygen supply, or the composition of the nutrient media, such as, for example, the sugar concentration during the fermentation, or the working up to the product, for example by ion exchange chromatography, or the intrinsic production properties of the microorganism itself. Bacteria of other genera such as Escherichia or Bacillus are also used for preparing amino acids.

A number of mutant strains producing a range of desirable compounds from the series of sulfur-containing fine chemicals have been developed by strain selection. The methods used to improve the production properties of these microorganisms in terms of the production of a particular molecule are those of mutagenesis, selection and choice of mutants. This is, however, a time-consuming and difficult process. EP-A-0 066 129 describes by way of example a process for preparing threonine using corynebacteria. Corresponding processes have also been elaborated for preparing methionine. In this way, for example, strains are obtained which are resistant to antimetabolites such as, for example, the methionine analogs α-methylmethionine, ethionine, norleucine, N-acetyinorleucine, S-trifluoromethylhomocysteine, 2-amino-5-heprenoit acid, selenomethionine, methionine sulfoximine, methoxine, 1-aminocyclopentanecarboxylic acid or auxotrophic for metabolites of regulatory importance and produce sulfur-containing fine chemicals such as, for example, L-methionine. Processes of this type developed for preparing methionine have the disadvantage that the yields are too low for economic utilization and they are therefore unable to compete with chemical synthesis.

Zeh et al. (Plant Physiol., Vol. 127, 2001: 792-802) describe an increase in the methionine content in potato plants through inhibition of threonine synthase by so-called antsense technology. This leads to a reduced activity of threonine synthase without reducing the threonine content in the plants. It is disadvantageous that this technology is very complicated and can be used only very poorly on an industrial scale, if at all. In addition, there must be highly differentiated inhibition of the enzymic activity because, otherwise, an auxotrophy for the amino acid occurs and the plant no longer grows.

Methods of recombinant DNA technology have likewise been employed for some years for strain improvement for L-amino acid-producing Corynebacterium strains by amplifying individual amino acid biosynthesis genes and examining the effect on amino acid production.

Amounts of amino acids exceeding the protein biosynthesis requirements of the cell cannot be stored and are instead degraded, so that intermediates are provided for the main metabolic pathways of the cell [for a review, see Stryer, L., Biochemistry, 3rd edition, Chapter 21 “Amino Acid Degradation and the Urea Cycle”; pages 495-516 (1988)]. Although the cell is able to convert unwanted amino acids into useful metabolic intermediates, amino acid production is costly in terms of energy, the precursor molecules and the enzymes necessary for their synthesis. It is therefore not surprising that amino acid biosynthesis is controlled by feedback inhibition, with the presence of a particular amino acid slowing down or entirely terminating its own production [for a review of the feedback mechanism in amino acid biosynthetic pathways, see Stryer, L., Biochemistry, 3rd edition, Chapter 24, “Biosynthesis of Amino Acids and Heme”, pages 575-600 (1988)]. The output of a particular amino acid is therefore restricted by the amount of this amino acid in the cell.

Improvements in the preparation of fine chemicals by fermentation usually correlate with improvements in substance fluxes and yields. It is important in this connection to prevent or reduce inhibition of important synthetic enzymes by intermediates or final products. It is likewise advantageous to prevent or reduce wastage of the carbon flux in unwanted products or side products.

The essential amino acids are, as described above, necessary for humans and many mammals, for example for domestic animals. L-Methionine is important in this connection as methyl group donor for the biosynthesis of, for example, choline, creatine, adrenaline, bases and RNA and DNA, histidine, and for transmethylation after formation of S-adenosylmethionine or as sulfhydryl group donor for cystene formation.

L-Methionine additionally appears to have a beneficial effect on depressions.

Improvement in the quality of human and animal foods is therefore an important task of the human and animal food industries. This is necessary because, for example, amino acids such as L-lysine and L-tryptophan in plants are limiting for the supply to mammals. An amino acid pattern which is as balanced as possible is particularly advantageous for the quality of human and animal foods, because a large excess of one amino acid such as, for example, L-lysine has, above a particular concentration in the foodstuff, no further beneficial effect on the utilization of the foodstuff, because other amino acids suddenly become limiting. A further increase in the quality is possible only by adding further amino acids which are limiting under these conditions. Thus, in growing pigs, lysine is initially limiting. If the food contains sufficient lysine, threonine becomes the limiting amino acid. If threonine is also added sufficiently to the food, the next limited amino acid is tryptophan. The sequence of the first three limiting amino acids for chickens is as follows: methionine, lysine and then threonine. This shows that these amino acids have an important function for optimal nutrition and must be present in a balanced ratio in the diet.

Great care is therefore necessary in specific dosage of the limiting amino acid in the form of synthetic products in order to avoid amino acid imbalances. This is because addition of an essential amino acid stimulates protein digestion, which may elicit in particular deficiency situations for limiting amino acid in second or third place.

Thus, in feeding trials for example of casein with additional doses of methionine, which is limited in casein, fatty degeneration of the liver has been found and could be eliminated only after additional dosage of tryptophan.

A balanced addition of a plurality of amino acids is therefore necessary for high quality of human and animal food, depending on the organism. The aforementioned fermentative and other synthetic processes usually make it possible to obtain only one amino acid.

It is an object of the present invention to develop a cost-effective process for synthesizing amino acids, advantageously the essential amino acids L-lysine and L-methionine, preferably L-methionine, which are among the two most common limiting amino acids.

We have found that this object is achieved by the process of the invention for preparing amino acids, advantageously L-methionine, in transgenic organisms, wherein the process comprises the following steps:
a) introduction of a nucleic acid sequence which codes for a threonine-degrading protein and/or lysine-degrading protein, or
b) introduction of a nucleic acid sequence which increases threonine degradation and/or lysine degradation in the transgenic organisms, and
c) expression of a nucleic acid sequence mentioned under (a) or (b) in the transgenic organism.

Threonine-degrading proteins advantageously mean proteins such as threonine aldolase (EC 4.1.2.5) or serine hydroxymethyltransferase (EC 2.1.2.1), which convert threonine into acetaldehyde and glycine, threonine dehydrogenase which converts threonine into L-2-aminoacetoacetate with formation of NADH+H⁺, or threonine dehydratase which converts threonine into oxobutyrate with elimination of NH₃ and water. Threonine aldolase is advantageously used as threonine-degrading activity in the process of the invention. The activity of the aforementioned proteins and/or of the nucleic acid sequences coding for them can be increased in various ways. The nucleic acid sequences are advantageously expressed in an organism, and thus the activity in an organism is increased via the gene copy number and/or else the stability of the expressed mRNA is increased and/or the stability of the gene product is increased. A further possibility is to change the regulation of the aforementioned nucleic acid sequences so that expression of the genes is increased. This can advantageously be achieved by heterologous regulatory sequences or by modifying, e.g. by mutation, the natural regulatory sequences present. It is also possible to combine the two advantageous methods together.

An advantageous embodiment of the process of the invention is a process for preparing amino acids, advantageously L-methionine, in transgenic organisms, which process comprises the following steps:

a) introduction of a nucleic acid sequence which codes for a threonine-degrading protein which comprises the following consensus sequence

H[x]₂G[X]R[X]₁₉D[X]₇K[X]₂₇G,
or
HXDGAR[X]₃A[X]₁₅D[X]₄CXSK[X]₄PXGS[X]₃G[X]₇A[X]₄
K[X]₂GGGXRQXG

b) introduction of a nucleic acid sequence which increases the threonine degradation in the transgenic organism, and
c) expression of a nucleic acid sequence mentioned under (a) or (b) in the transgenic organism.

Where the one letter amino acid code has been used in the consensus sequence. Any amino acid may be present at places where there is an X. FIG. 1 represents the consensus of threonine aldolase which are able advantageously to be used in the process of the invention.

The abbreviations of the proteins and their accession numbers mentioned in FIG. 1 mean the following: P1; T24108 is a hypothetical protein R102.4b from Caenorhabditis elegans, Q87110 is a putative L-allo-threonine aldolase from Vibrio parahaemolyticus, GLY1_YEAST is a low-specificity L-threonine aldolase from Saccharomyces cerevisiae (Baker's yeast), P1; T38302 is a possible threonine aldolase from Schizosaccharomyces pombe, P1; E75410 is an L-allo-threonine aldolase from Deinococcus radiodurans. Q9VCK6 is referred to as CG10184 protein and is derived from Drosophila melanogaster (fruit fly), Q885J1 is a low-specificity threonine aldolase from Pseudomonas syringae. P1; G83533 is a hypothetical protein PA0902 from Pseudomonas aeruginosa, Q83S08 is a putative arylsulfatase from Shigella flexneri, P1; F64825 is an L-allo threonine aldolase from Escherichia coli, as is P1; AF0608, which is derived from Salmonella enterica. Q87HF4 is an L-allo-threonine aldolase from Vibrio parahaemolyticus. The following proteins are also L-allo-threonine aldolases: P1; E82418 (Vibrio cholerae), P1; T46877 (Aeromonas jandaei), Q9M835 (Arabidopsis thaliana; mouse-ear cress), Q8RCY7 (Thermoanaerobacter tengcongensis), P1; C72215 (Thermotoga maritima), Q896G8 (Clostridium tetani), P1; C84060 (Bacillus halodurans). Q89N26 is a B114016 protein from Bradyrhizobium japonicum. Q9X8S4 is a low-specificity L-threonine aldolase from Zymomonas mobilis, P1; D84395 is an L-allo-threonine aldolase from Halobacterium sp. NRC-1. CAA02484 is sequence 33 from the PCT application WO 94/25606. The sequences derived from Tolypocladium inflatum. TOXG_COCCA is an alanine racemase TOXG from Cochliobolus carbonum (Bipolaris zeicola), P1; AF1474 is derived from Listeria innocua and is a low-specificity L-allo-threonine aldolase.

Lysine-degrading proteins advantageously mean proteins such as lysine decarboxylase (EC 4.1.1.18), L-lysine 6-monooxygenase (EC 1.14.13.59), L-lysine 2-monooxygenase (EC 1.13.12.2), lysine ketoglutarate reductase (EC 1.5.1.7) or lysine 2,3-aminomutase (EC 5.4.3.2), which convert L-lysine into cadaverine, N6-hydroxy-L-lysine, 5-aminopentanamide, saccharopin or (3S)-3,6-aminohexanoate. The lysine-degrading activity advantageously used in the process of the invention is lysine decarboxylate alone or in combination with a threonine-degrading activity, advantageously of threonine aldolase. The activity of the aforementioned proteins and/or of the nucleic acid sequences coding for them can be increased in various ways. The nucleic acid sequences are advantageously expressed in an organism, and thus the activity in an organism is increased via the gene copy number and/or else the stability of the expressed mRNA is increased and/or the stability of the gene product is increased. A further possibility is to alter the regulation of the aforementioned nucleic acid sequences so that the expression of the genes are altered so that the expression of the genes is increased. This can advantageously be achieved by heterologous regulatory sequences or by modification, e.g. by mutation of the natural regulatory sequences which are present. It is also possible for the two advantageous methods to be combined together.

One advantageous embodiment of the process of the invention is a process for preparing amino acids, advantageously L-methionine, in transgenic organisms, which process comprises the following steps:

a) introduction of a nucleic acid sequence which codes for a lysine-degrading protein which comprises the following consensus sequence

G[X]₄GIM[X]₄₅M[X]₂RK[X]₂M[X]₁₁GGXG[X]₃E[X]₂E[X]₃W,
or
LG[X]₉LVYGG[X]₃GIMGXVA[X]₉G[X]₃GXIP[X]₂₄MHXRK[X]₂
M[X]₆F[X]₃PGGXGTXEE[X]₂E[X]₂TW[X]₂IG[X]₃
KP[X]₄N[X]₃FY[X]₁₄F

b) introduction of nucleic acid sequence which increases the lysine degradation in the transgenic organism, and
c) expression of a nucleic acid sequence mentioned under (a) or (b) in the transgenic organism.

Where the one letter amino acid code has been used in the consensus sequence. Any amino acid may be present at places where there is an X. FIG. 2 represents the consensus of the lysine-degrading protein which are able advantageously to be used in the process of the invention. The amino acid sequences listed in FIG. 2 are numbered (1, 2, 3, etc.) and denote the following abbreviations of the proteins or their accession numbers: Q871Q6 [2] is a hypothetical protein from Neurospa crassa. Q815T3 [3] is a lysine decarboxylase from Bacillus cereus. Q81XE4 [4] codes for a hypothetical protein from Bacillus anthracis, just like P1; D70033 [5] codes for a hypothetical protein from Bacillus subtilis. Q8H7U8 [6] is a putative lysine decarboxylase from Oryza sativa. Q8L8B8 [7] codes for a hypothetical protein from Arabidopsis thaliana. Q8XXM6 [8] is also a hypothetical protein from Ralstonia solanacearum. The following proteins are also hypothetical proteins: Q88DF4 [9] (Pseudomonas putida), P1; A83031 [10] (Pseudomonas aeruginosa), Q8PAJ9 [11] (Xanthomonas campestris) and Q8PMA0 [12] (Xanthomonas axonopodis). Q8NN34 [13] codes for a protected Rossmann fold nucleotide binding protein (1 segment) from Corynebacterium glutamicum. P1; A13438 [14] codes for a lysine decarboxylase from Brucella melitensis. Q8G289 [15] codes for a hypothetical protein from Brucella suis, as does Q984W8 [16] (Rhizobium loti). P1; B97490 [17] is a lysine decarboxylase from Agrobacterium tumefaciens. P1; B83993 [18] also codes for a lysine decarboxylase from Bacillus halodurans. Q8A2T1 [19] is assumed to be a putative lysine decarboxylase from Bacteroides thetaiotaomicron. The following proteins code for hypothetical proteins: Q92R13 [20] (Rhizobium meliloti), Q8ETC2 [21] (Oceanobacillus iheyensis), Q8NXQ6 [22] (Staphylococcus aureus), Q8CTK0 [23] (Staphylococcus epidermis) and P1; F55578 [24] (Rhodococcus fascians). Q839D0 [25] codes for a protein of the decarboxylase family from Enterococcus faecalis. P1; D84035 [26] is a hypothetical protein from Bacillus halodurans. Q8EZ03 [27] is a lysine decarboxylase from Leptospira interrogans. Q89NP4 [28] is a hypothetical protein from Bradyrhizobium japonicum, as is Q8RFZ1 [29] (Fusobacterium nucleotum). The numbers in square brackets indicate the numbering shown in FIG. 2 and thus the sequence of the proteins. The clone YJL055w which is advantageously used in the process of the invention is given the number 1. The consensus sequence is shown in number 30.

In a further embodiment of the process, it is a process for preparing amino acids, advantageously L-methionine, in transgenic organisms, which process comprises the following steps:

a) introduction of a nucleic acid sequence which codes for a threonine-degrading protein which comprises the following consensus sequence

H[x]₂G[X]R[X]₁₉D[X]₇K[X]₂₇G,
or
HXDGAR[X]₃A[X]₁₅D[X]₄CXSK[X]₄PXGS[X]₃G[X]₇A[X]₄K[X]₂
GGGXRQXG

and introduction of a nucleic acid sequence which codes for a lysine-degrading protein which comprises the following consensus sequence

G[X]₄GIM[X]₄₅M[X]₂RK[X]₂M[X]₁₁GGXG[X]₃E[X]₂E[X]₃W,
or
LG[X]₉LVYGG[X]₃GIMGXVA[X]₉G[X]₃GXIP[X]₂₄MHXRK[X]₂M[X]₆F[X]₃
PGGXGTXEE[X]₂E[X]₂TW[X]₂IG[X]₃KP[X]₄N[X]₃FY[X]₁₄F,
or

b) introduction of a nucleic acid sequence which codes for proteins which increase threonine degradation and lysine degradation in the transgenic organisms, and
c) expression of a nucleic acid sequence mentioned under (a) or (b) in the transgenic organism.

In an advantageous embodiment of the process for preparing amino acids, advantageously L-methionine, in transgenic organisms, the process comprises introducing in the abovementioned process step (a) a nucleic acid sequence which is selected from the group of nucleic acid sequences
i) of a nucleic acid sequence having the sequence depicted in SEQ ID NO: 1; SEQ ID NO: 11; SEQ ID NO: 13; SEQ ID NO: 15; SEQ ID NO: 17; SEQ ID NO: 19; SEQ ID NO: 21; SEQ ID NO: 23 or SEQ ID NO: 25;
ii) of a nucleic acid sequence obtained owing to the degeneracy of the genetic code through back-translation of the amino acid sequence depicted in SEQ ID NO: 2, SEQ ID NO: 12; SEQ ID NO: 14; SEQ ID NO: 16; SEQ ID NO: 18; SEQ ID NO: 20; SEQ ID NO: 22; SEQ ID NO: 24 or SEQ ID NO: 26 and
iii) of a derivative of the nucleic acid sequence depicted in SEQ ID NO: 1, SEQ ID NO: 11; SEQ ID NO: 13; SEQ ID NO: 15; SEQ ID NO: 17; SEQ ID NO: 19; SEQ ID NO: 21; SEQ ID NO: 23 or SEQ ID NO: 25; which codes for a polypeptide having at least 50% homology at the amino acid level with the amino acid sequence depicted in SEQ ID NO: 2, SEQ ID NO: 12; SEQ ID NO: 14; SEQ ID NO: 16; SEQ ID NO: 18; SEQ ID NO: 20; SEQ ID NO: 22; SEQ ID NO: 24 or SEQ ID NO: 26 with a negligible reduction in the biological activity of the polypeptides; and
   subsequently expressing these nucleic acid sequences in a transgenic organism.

A further advantageous embodiment of the process for preparing amino acids, advantageously L-methionine, in transgenic organisms is the process wherein in the abovementioned process step (a) or (b) nucleic acid sequences in combination with one another or in combination with other nucleic acid sequences which are able to increase the synthesis of L-lysine and/or I-threonine in a transgenic organism. These include, besides genes which code for central metabolisms such as the utilization of sugars such as glucose within glycolysis or the citrate cycle, also genes which, starting from aspartate, are involved in the synthesis of amino acids.

This advantageous embodiment of the process for preparing amino acids, advantageously L-methionine, in transgenic organisms thus appears as follows:
a) introduction of a nucleic acid sequence selected from the group
   i) of a nucleic acid sequence having the sequence depicted in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 and/or SEQ ID NO: 25;
   ii) of a nucleic acid sequence obtained owing to the degeneracy of the genetic code through back-translation of the amino acid sequence depicted in SEQ ID NO: 2, SEQ ID NO: 12, SEQ ID NO: 14, SEQ ID NO: 16, SEQ ID NO: 18, SEQ ID NO: 20, SEQ ID NO: 22, SEQ ID NO: 24 and/or SEQ ID NO: 26, and
   iii) of a derivative of the nucleic acid sequence depicted in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 and/or SEQ ID NO: 25; which codes for a polypeptide having at least 50% homology at the amino acid level with the amino acid sequence depicted in SEQ ID NO: 2, SEQ ID NO: 12, SEQ ID NO: 14, SEQ ID NO: 16, SEQ ID NO: 18, SEQ ID NO: 20, SEQ ID NO: 22, SEQ ID NO: 24 and/or SEQ ID NO: 26 with a negligible reduction in the biological activity of the polypeptides; and
b) expression of a nucleic acid sequence mentioned under (a) in a transgenic organism.

In a further advantageous embodiment of the process for preparing amino acids, advantageously L-methionine, in transgenic organisms, the process comprises introducing in the abovementioned process step (a) one or more nucleic acid sequences which are selected from the group of nucleic acid sequences
i) of a nucleic acid sequence obtained owing to the degeneracy of the genetic code through back-translation of the amino acid sequence depicted in SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10;
ii) of a derivative of the nucleic acid sequence which is obtained by back-translation of the amino acid sequence depicted in SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 and which has at least 70% homology at the amino acid level with the aforementioned amino acid sequences, with a negligible reduction in the biological activity of the polypeptides; and
   subsequently expressing these nucleic acid sequences in a transgenic organism.

After the introduction and expression of the nucleic acid sequences used in the processes of the invention, the transgenic organism is advantageously cultured and subsequently harvested. In the case where the transgenic organism is a microorganism such as a eukaryotic organism such as a fungus, an alga or a yeast or a prokaryotic organism such as a bacterium such as a bacterium of the genera Escherichia, Bacillus, Serratia, Salmonella, Klebsiella, Enterobacter, Corynebacterium or Brevibacterium, the latter is cultured in a solid or liquid medium known to the skilled worker and usual for the particular organism. After culturing, the organisms are harvested where appropriate. The amino acids can then be further processed directly in the human or animal food or for other applications, for example as disclosed in EP-B-0 533 039 or EP-A-0 615 693, which are incorporated herein by reference, or else further purified in a conventional way by extraction and precipitation or crystallization or on an ion exchanger or combinations of these methods. Products of these various workups are amino acids or amino acid compositions still containing portions of the fermentation broth and of the cells in various amounts advantageously in the range from 0 to 100% by weight, preferably from 1 to 80% by weight, particularly preferably between 5 and 50% by weight, very particularly preferably between 5 and 40% by weight

In an advantageous embodiment of the invention, the organism is a plant whose amino acid content is advantageously modified by the introduced nucleic acid sequence. This is important for plant breeders because, for example, the nutritional value of plants for monogastric animals is limited by some essential amino acids such as lysine or methionine. Threonine also plays an important role in this connection. This transgenic plant produced in this way is, after introduction of the nucleic acid or nucleic acid combination used in the process of the invention, grown on or in a nutrient medium or else in solid culture, e.g. a soil culture and subsequently harvested. The plants can then be used directly as human or animal foods or else be further processed. It is also possible in this case to purify the amino acids further in a conventional way by extraction, crystallization and/or precipitation or on an ion exchanger or combination of these methods. Products of these various workups are amino acids or amino acid compositions still containing portions of the plant in various amounts advantageously in the range from 0 to 100% by weight, preferably from 20 to 80% by weight, particularly preferably between 50 and 90% by weight, very particularly preferably between 80 and 99% by weight. The plants are advantageously used immediately without further workup.

In a further embodiment of the invention, the organism is a microorganism such as bacteria of the genera Corynebacterium, Brevibacterium, Escherichia, Serratia, Salmonella, Klebsiella, Enterobacter or Bacillus. Microorganisms of the genera Corynebacterium, Brevibacterium, Escherichia or Bacillus are preferably used. These microorganisms are advantageously used in a fermentation process.

Besides the sequence specified in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 and/or SEQ ID NO: 25, the nucleic acid sequences which can be derived from the sequences SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10, or derivatives thereof, it is advantageous also for other genes to be expressed and/or mutated in the organisms. It is particularly advantageous for at least one further gene of the L-lysine, L-threonine and/or L-methionine biosynthetic pathway additionally to be expressed in the organisms such as plants or microorganisms, and/or for genes whose regulation have been modified to be expressed. It is also possible and advantageous to have modified the regulation of the natural genes in such a way that the gene and/or its gene product is no longer subject to the control systems present in the organisms. This results in enhanced synthesis of the desired amino acids because, for example, feedback regulation is no longer present or is no longer present to the same extent. The process of the invention advantageously produces amino acids such as L-lysine, L-threonine and/or L-methionine, preferably L-methionine.

In a further embodiment of the process of the invention, therefore, organisms are grown, advantageously bacteria of the genera Corynebacterium, Brevibacterium, Bacillus or Escherichia or plants, in which there is simultaneous overexpression of at least one nucleic acid or one of the genes which code for proteins selected from the group of gene products consisting of aspartate kinase (lysC), of aspartate-semialdehyde dehydrogenase (asd), of dihydrodipicolinate synthase, of dihydrodipicolinate reductase, of tetrahydrodipicolinate succinyltransferase, of N-succinyl-L-diaminopimelinate glutamate transaminase, of succinyl diaminopimelate desuccinylase, of diaminopimelate epimerase, of diaminopimelate decarboxylase, of glyceraldehyde-3-phosphate dehydrogenase (gap), of 3-phosphoglycerate kinase (pgk), of pyruvate carboxylase (pyc), of triosephosphate isomerase (tpi), of homoserine O-acetyltransferase (metA), of cystathionine γ-synthase (metB), of cystathionine gamma-lyase (metC), cystathionine β-lyase, of methionine synthase (metH), of serine hydroxymethyltransferase (glyA), of O-acetylhomoserine sulfhydrylase (metY), of methylenetetrahydrofolate reductase (metF), of phosphoserine aminotransferase (serC), of phosphoserine phosphatase (serB), of serine acetyltransferase (cysE), of cysteine synthase (cysK), of homoserine dehydrogenase (hom), homoserine kinase, homocystene S-methylransferase and S-adenosylmethionine synthase (metX).

In a further advantageous embodiment of the process of the invention, the organisms used in the process are those in which simultaneously at least one of the aforementioned genes or one of the aforementioned nucleic acids is mutated so that the activity of the corresponding proteins is influenced by metabolites to a smaller extent compared with the unmutated proteins, or not at all, and that in particular the production according to the invention of the amino acids is not impaired, or so that their specific enzymatic activity is increased. Little influence means in this connection that the regulation of the enzymic activity is less by at least 10%, advantageously at least 20, 30 or 40%, particularly advantageously by at least 50, 60 or 70%, compared with the starting organism, i.e. influence on their enzymatic activity by metabolites and thus the activity of the enzyme is increased by these figures mentioned compared with the starting organism. An increase in the enzymatic activity means an enzymatic activity which is increased by at least 10%, advantageously at least 20, 30 or 40%, particularly advantageously by at least 50, 60 or 70%, compared with the starting organism. This leads to an increased productivity of the desired amino acid or of the desired amino acids.

In a further advantageous embodiment of the process of the invention, the organisms used in the process are those in which simultaneously at least one of the genes which codes for an enzymatic activity selected from homoserine kinase (thrB), threonine dehydratase (ilvA), threonine synthase (thrC), meso-diaminopimelate D-dehydrogenase (ddh), phosphoenolpyruvate carboxykinase (pck), glucose-6-phosphate 6-isomerase (pgi), pyruvate oxidase (poxB), dihydrodipicolinate synthase (dapA), dihydrodipicolinate reductase (dapB) and diaminopicolinate decarboxylase (lysA) is attenuated, in particular by reducing the rate of expression of the corresponding gene.

In another embodiment of the process of the invention, the organisms used in the process are those in which simultaneously at least one of the aforementioned nucleic acids or of the aforementioned genes is mutated in such a way that the enzymatic activity of the corresponding protein is partially reduced or completely blocked. A reduction in the enzymatic activity means an enzymatic activity which is reduced by at least 10%, advantageously at least 20, 30 or 40%, particularly advantageously by at least 50, 60 or 70%, compared with the starting organism.

The activity of enzymes can be influenced in such a way that there is a reduction or increase in the reaction rate, or a modification (reduction or increase) in the affinity for the substrate.

Microorganisms of the genera Corynebacterium or Brevibacterium or plants are preferably employed in the process of the invention.

It is also possible to prepare chemically pure amino acids or amino acid compositions by the processes described above. For this purpose, the amino acids or the amino acid compositions are isolated from the organism such as the microorganisms or the plants or the culture medium in which or on which the organisms have grown, or from the organism and the culture medium, in a known manner. These chemically pure amino acids or amino acid compositions are advantageous for applications in the food industry, the cosmetics industry or the drugs industry sectors.

Amino acids such as methionine, lysine or mixtures thereof, preferably methionine, are advantageously prepared by the process of the invention.

It is moreover possible to increase the aforementioned amino acids in the process of the invention by at least a factor of 3, preferably by at least a factor of 5, particularly preferably by at least a factor of 10, very particularly preferably by at least a factor of 50, compared with the wild type of the organisms. There is a particularly advantageous effect on the amino acid productivity in the process of the invention if a combination of genes which code for a threonine aldolase or threonine aldolase-like protein or a lysine decarboxylase or lysine decarboxylase-like protein is used.

It is possible in principle to increase by the process of the invention the amino acids prepared in the organisms used in the process in two ways. It is possible advantageously to increase the pool of free amino acids and/or the proportion of amino acids prepared by the process in the proteins. The process of the invention advantageously increases the pool of free amino acids in the transgenic organisms. In the advantageous case of fermentation of microorganisms, the amino acids are enriched in the medium.

Suitable in principle for the process of the invention are all eukaryotic or prokaryotic organisms able to synthesize methionine and/or lysine. The organisms used in the process are advantageously microorganisms such as bacteria, fungi, yeasts or algae or plants such as dicotyledonous or monocotyledonous plants such as plants of the Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, lridaceae, Liliaceae, Orchidaceae, Gentianaceae, Labiaceae, Magnoliaceae, Ranunculaceae, Caprifolaceae, Rubiaceae, Scrophulariaceae, Caryophyllaceae, Ericaceae, Polygonaceae, Violaceae, Juncaceae or Poaceae families, preferably a plant selected from the group of families Apiaceae, Asteraceae, Brassicaceae, Cucurbitaceae, Fabaceae, Papaveraceae, Rosaceae, Solanaceae, Liliaceae or Poaceae.

It is advantageous to use in the process of the invention transgenic microorganisms such as fungi such as the genus Claviceps or Aspergillus or Gram-positive bacteria such as the genera Bacillus, Corynebacterium, Micrococcus, Brevibacterium, Rhodococcus, Nocardia, Caseobacter or Arthrobacter or Gram-negative bacteria such as the genera Escherichia, Flavobacterium or Salmonella or yeasts such as the genera Rhodotorula, Hansenula or Candida. Particularly advantageous organisms are selected from the group of genera Corynebacterium, Brevibacterium, Escherichia, Bacillus, Serraba, Salmonella, Klebsiella, Enterobacter, Rhodotorula, Hansenula, Candida, Claviceps or Flavobacterium. It is very particularly advantageous to use in the process of the invention microorganisms selected from the group of genera and species consisting of Hansenula anomala, Candida utilis, Claviceps purpurea, Bacillus circulans, Bacillus subtilis, Bacillus sp., Brevibacterium albidum, Brevibacterium album, Brevibacterium cerinum, Brevibacterium flavum, Brevibacterium glutamigenes, Brevibacterium iodinum, Brevibacterium ketoglutamicum, Brevibacterium lactofermentum, Brevibacterium linens, Brevibacterium roseum, Brevibacterium saccharolyticum, Brevibacterium sp., Corynebacterium acetoacidophilum, Corynebacterium acetoglutamicum, Corynebacterium ammoniagenes, Corynebacterium glutamicum (=Micrococcus glutamicum), Corynebacterium melassecola, Corynebacterium sp. or Escherichia coli, specifically Escherichia coli K12 and its described strains.

It is advantageous to use in the process of the invention transgenic plants selected from the group of useful plants. Such as plants selected from the group of peanut, oilseed rape, canola, sunflower, safflower, olive, sesame, hazelnut, almond, avocado, bay, pumpkin, flax, soybean, pistachio, borage, corn, wheat, rye, oats, millet, triticale, rice, barley, cassaya, potato, sugar beet, feed beet, aubergine, and perennial grasses and feed crops, oil palm, vegetables (brassicas, roots, tubers, legumes, fruit vegetables, bulbs, leaf and stem vegetables), buckwheat, Jerusalem artichoke, broad bean, vetches, lentil, dwarf bean, alfalfa, lupin, clover and luceme.

The nucleic acid sequence(s) used in the process for preparing amino acids in transgenic organisms are advantageously derived from a eukaryote (the plural is intended to include the singular and vice versa for the invention), but may also be derived from a prokaryote such as bacteria selected from the genera Brevibacterium, Escherichia, Salmonella, Bacillus, Corynebacterium, Serratia, Klebsiella or Enterobacter. The nucleic acid sequences are advantageously derived from a plant such as a plant selected from the Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, Iridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae families, preferably a plant selected from the group of families Apiaceae, Asteraceae, Brassicaceae, Cucurbitaceae, Fabaceae, Papaveraceae, Rosaceae, Solanaceae, Liliaceae or Proaceae, a fungus such as the genera Aspergillus, Penicillum or Claviceps or a yeast such as the genera Pichia, Torulopsis, Hansenula, Schizosaccharomyces, Candida, Rhodotorula or Saccharomyces. The sequences are particularly advantageously derived from yeasts such as the genera Pichia, Torulopsis, Hansenula, Schizosaccharomyces, Candida, Rhodotorula or Saccharomyces, very particularly advantageously from the yeast of the Saccharomycetaceae family such as the advantageous genus Saccharomyces and the particularly advantageous genus and species Saccharomyces cerevisiae.

The nucleic acid sequences used in the process of the invention and having the sequence SEQ ID NO: 1, SEQ ID NO: 13 and/or SEQ ID NO: 15 code for a threonine aldolase. This aldolase (SEQ ID NO: 1) shows the highest homology with the GLY1 protein from A. gossypii [Eremothecium ashbii, Eremothecium gossypii] (EMBL database accession No. AJ005442, CM06545.1, GENSEQ_PROT: AAY25338, identity at the amino acid level with SEQ ID NO: 1 of 76%). There is also a high degree of homology with threonine aldolases derived from rice, soybean, wheat and disclosed in US 2002123118 A1. Homologies with a large number of nucleic acids can additionally be found. The threonine aldolase from yeasts such as Candida albicans (EMBL database accession No. AF009967, AAB64198.1, identity at the amino acid level with SEQ ID NO: 1 of 56%), from Schizosaccharomyces pombe (EMBL database accession No. Z99163, CAB16235.1, identity at the amino acid level with SEQ ID NO: 1 of 49%), or from bacteria such as Aeromonas jandaei (EMBL database accession No. AF169478, AAD47837.1, identity at the amino acid level with SEQ ID NO: 1 of 41%), Pseudomonas aeruginosa (EMBL database accession No. AF011922, AAC46016.1, identity at the amino acid level with SEQ ID NO: 1 of 38%), Vibrio cholerae (EMBL database accession No. AE004405, AAF96663.1, identity at the amino acid level with SEQ ID NO: 1 of 38%), Escherichia coli (EMBL database accession No. AB005050, BAA20882.1, identity at the amino acid level with SEQ ID NO: 1 of 38%), Deinococcus radiodurans (EMBL database accession No. AE001978, AAF10885.1, identity at the amino acid level with SEQ ID NO: 1 of 38%), Bacillus halodurans (EMBL database accession No. AP001518, BAB07002.1, identity at the amino acid level with SEQ ID NO: 1 of 34%), Halobacterium sp. (EMBL database accession No. AE005124, AAG20528:1), Thermotoga maritima (EMBL database accession No. AE001813, AAD36809.1, identity at the amino acid level with SEQ ID NO: 1 of 40%) or the plants Arabidopsis thaliana (EMBL database accession No. AF325033, AAG40385.1, AC022287, AAF63783.1, AC003981, AAC14037.1, identity at the amino acid level with SEQ ID NO: 1 of in each case 40, 42 or 37%) or from nonhuman animals such as Caenorhabditis elegans (EMBL database accession No. Z70309, CM94358.1, identity at the amino acid level with SEQ ID NO: 1 of 41%) or Drosophila melanogaster (EMBL database accession No. AE003744, MF56152.1, identity at the amino acid level with SEQ ID NO: 1 of 39%) or the alanine racemase from fungi such as Cochliobolus carbonum/Bipolaris zeicola (EMBL database accession No. AF169478, AAD47837.1, identity at the amino acid level with SEQ ID NO: 1 of 38%). It is advantageous to use in the process of the invention nucleic acid sequences and proteins encoded thereby which are derived from yeasts of the genera Candida, Hansenula, Rhodotorula, Schizosaccharomyces or Saccharomyces. The aldolase which is advantageously used in the process of the invention additionally shows high homology with the sequences which are specified in SEQ ID NO: 3 (identity at the amino acid level with SEQ ID NO: 1 of 35%), SEQ ID NO: 4 (identity at the amino acid level with SEQ ID NO: 1 of 35%), SEQ ID NO: 5 (identity at the amino acid level with SEQ ID NO: 1 of 27%), SEQ ID NO: 6 (identity at the amino acid level with SEQ ID NO: 1 of 43%), SEQ ID NO: 7 (identity at the amino acid level with SEQ ID NO: 1 of 39%), SEQ ID NO: 8 (identity at the amino acid level with SEQ ID NO: 1 of 32%), SEQ ID NO: 9 (identity at the amino acid level with SEQ ID NO: 1 of 35%) or SEQ ID NO: 10 (identity at the amino acid level with SEQ ID NO: 1 of 36%) and which are derived from soybean (SEQ ID NO: 3-5), rice (SEQ ID NO: 6 and 7) and from canola (SEQ ID NO: 8-10). It is possible and advantageous to use in the process nucleic acid sequences derived from the amino acid sequences SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

The nucleic acid sequences used in the process of the invention and having the sequence of SEQ ID NO: 11, SEQ ID NO: 17, SEQ ID NO: 19, SEQ ID NO: 21, SEQ ID NO: 23 or SEQ ID NO: 25 and derivatives thereof code for a lysine decarboxylase or for a lysine decarboxylase-like protein. This advantageous lysine decarboxylase (SEQ ID NO: 11) shows the highest homology with a protein from Neurospora crasa (EMBL database. Accession No. TREMBL_NEW:CAD70937, identity at the amino acid level with SEQ ID NO: 11 of 45% over a maximum length of 231 amino acids=AA). The lysine decarboxylases from Oryza sative (EMBL database. Accession No. SPTREMBL:Q9FWG6, TREMBL_NEW:AA019380, show identity at the amino acid level with SEQ ID NO: 11 of respectively 41% and 37%) and the proteins from Arabidopsis thaliana (EMBL database. Accession No. SPTREMBL:Q9ASW6, PIR:T45885, SPTREMBL:Q9FNH8, PIR:H84789, PIR:T48348, SPTREMBL:Q9FYM7, PIR:T48554, PIR:T04966 and PIR:E84775, identity at the amino acid level with SEQ ID NO: 11 respectively of 42%, 42%, 41%, 39%, 39%, 39%, 39%, 39% and 35%) or from bacteria such as Ralstonia solanacearum [=Pseudomonas solanacearum] (EMBL database. Accession No. SPTREMBL:Q8XXM6, identity at the amino acid level with SEQ ID NO: 11 of 43%), Pseudomonas putida (EMBL database. Accession No. TREMBL_NEW:AAN70440, identity at the amino acid level with SEQ ID NO: 11 of 40%), Pseudomonas aeruginosa (EMBL database. Accession No. PIR:A83031, identity at the amino acid level with SEQ ID NO: 11 of 40%), Bacteroides thetaiotaomicron (EMBL database. Accession No. TREMBL_NEW:AA078330, identity at the amino acid level with SEQ ID NO: 11 of 39%), Brucella melitensis (EMBL database. Accession No. PIR:AI3438, identity at the amino acid level with SEQ ID NO: 11 of 43%), Bacillus subtilis (EMBL database. Accession No. PIR:D70033, identity at the amino acid level with SEQ ID NO: 11 of 36%), Rhizobium loti or Rhisobium meliloti (EMBL database. Accession No. STREMBL:Q984W8 or STREMBL:Q92R13, identity at the amino acid level with SEQ ID NO: 11 of 39% and 36% respectively), Bacillus halodurans. (EMBL database. Accession No. PIR:B83993, identity at the amino acid level with SEQ ID NO: 11 of 37%), Agrobacterium tumefaciens (EMBL database. Accession No. PIR:AI2707 or PIR:B97490, identity at the amino acid level with SEQ ID NO: 11 of respectively 41%), Staphylococcus aureus (EMBL database. Accession No. PIR:A89839, identity at the amino acid level with SEQ ID NO: 11 of respectively 34%), also show homologies with the lysine decarboxylase sequence SEQ ID NO: 11 used according to the invention. It is advantageous to use in the process of the invention nucleic acid sequences and proteins encoded thereby which are derived from yeasts of the genera Candida, Hansenula, Rhodotorula, Schizosaccharomyces or Saccharomyces. The lysine decarboxylase which is advantageously used in the process of the invention additionally shows high homology with those under SEQ ID NO: 21 (identity at the amino acid level with SEQ ID NO: 11 of 42%), SEQ ID NO: 23 (identity at the amino acid level with SEQ ID NO: 11 of 43%) or SEQ ID NO: 25 (identity at the amino acid level with SEQ ID NO: 11 of 37%) which are derived from oilseed rape, rice and maize. It is possible and advantageous to use in the process nucleic acid sequences derived from the amino acid sequences SEQ ID NO: 11, SEQ ID NO: 17, SEQ ID NO: 19, SEQ ID NO: 21, SEQ ID NO: 23 or SEQ ID NO: 25 and having lysine decarboxylase activity.

Nucleic acid sequences which are advantageous for the process of the invention and which code for polypeptides having threonine aldolase activity or lysine decarboxylase activity can be found in generally accessible databases. Particular mention should be made in this connection of general gene databases such as the EMBL database (Stoesser G. et al., Nucleic Acids Res 2001, Vol. 29, 17-21), of the GenBank database (Benson D. A. et al., Nucleic Acids Res 2000, Vol. 28, 15-18), or the PIR database (Barker W. C. et al., Nucleic Acids Res. 1999, Vol. 27, 39-43).

It is additionally possible to use organism-specific gene databases for finding advantageous sequences, e.g. advantageously for yeast the SGD database (Cherry J. M. et al., Nucleic Acids Res. 1998, Vol. 26, 73-80) or the MIPS database (Mewes H. W. et al., Nucleic Acids Res. 1999, Vol. 27, 44-48), for E coli the GenProtEC database (http://web.bham.ac.uk/bcm4ght6/res.html), for Arabidopsis the TAIR database (Huala, E. et al., Nucleic Acids Res. 2001 Vol. 29(1), 102-5) or the MIPS database.

In order to improve the introduction of the nucleic acid sequences and the expression of the sequences in the transgenic organisms used in the process, the nucleic acid sequences are inserted into a nucleic acid construct and/or a vector. In addition to the sequences described above and used in the process of the invention, further nucleic acid sequences, advantageously of biosynthesis genes of the amino acid prepared in the process, may be present in the nucleic acid construct or in the vector and are inserted together into the organism. These additional sequences may, however, also be inserted directly or via other separate nucleic acid constructs or vectors into the organisms. It is advantageous to introduce genes coding for threonine aldolases or lysine decarboxylase, alone or in combination, into an organism, advantageously a microorganism or a plant.

The nucleic acid sequences used in the process of the invention are isolated nucleic acid sequences coding for polypeptides having threonine aldolase activity or lysine decarboxylase activity.

Nucleic acids mean in the process of the invention DNA or RNA sequences which may be single- or double-stranded or may, where appropriate, have synthetic, unnatural or modified nucleotide bases which can be incorporated in DNA or RNA.

The term “expression” means the transcription and/or translation of a codogenic gene segment or gene. The resulting product is usually a protein. However, the products also include functional RNAs such as, for example, ribozymes. Expression may take place systemically or locally, e.g. confined to particular cell types, tissues or organs.

The expression products of the nucleic acids used in the process of the invention, e.g. of the codogenic gene segments (ORFs) and of their regulatory elements, can be characterized by their function. Included in this are, for example, functions in the areas of metabolism, energy, transcription, protein synthesis, protein processing, cellular transport and transport mechanisms, cellular communication and signal transduction, cell rescue, cell defense and cell virulence, regulation of the cellular environment and interaction of the cell with its environment, cell fate, transposable elements, viral proteins and plasmid proteins, cellular organization monitoring, subcellular localization, regulation of protein activity, proteins with binding function or cofactor requirement and transport facilitation. Genes of identical function are combined to so-called functional gene families.

It is possible through the biological activity of the nucleic acids which are used in the process of the invention and which code for polypeptides having threonine aldolase activity or lysine decarboxylase activity for different amino acids to be prepared or the preparation thereof to be improved and/or increased. Mixtures of the various amino acids can be prepared, depending on the selection of the organism used for the process of the invention, for example a microorganism or a plant. There is advantageously preparation of L-lysine and/or L-methionine as amino acid or amino acid mixture in the process of the invention. L-methionine is particularly preferably prepared in the process. These prepared amino acids may be present in the cells of the transgenic organisms as free amino acids and/or bound in proteins.

Transgenic organisms in the process of the invention mean when plants are concerned also plant cells, tissues, organs such as root, shoot, stem, seed, flower, tuber or leaf or whole plants grown to prepare amino acids. Growing means, for example; culturing the transgenic plant cells, tissues or organs on or in the nutrient medium or the whole plant on or in a substrate, for example in hydroculture, flowerpot soil or on a field.

If plants are chosen as donor organism in the process of the invention, it is possible in principle for this plant to have any phylogenetic relationship with the recipient plant. Thus, donor and recipient plants may belong to the same family, genus, species, variety or line, with the homology between the nucleic acids to be integrated and corresponding parts of the genome of the recipient plant increasing. The same also applies to microorganisms as donor and recipient organisms.

It is advantageous to use in the process of the invention a nucleic acid sequence having the sequence depicted in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 and/or SEQ ID NO: 25, nucleic acid sequences derived from amino acid sequences SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10, or derivative thereof or homologs which code for polypeptides which still have the enzymatic activity or biological activity. These sequences are cloned singly or in combination into expression constructs. These expression constructs make optimal synthesis of the amino acids produced in the process of the invention possible.

In a preferred embodiment, the process additionally includes the step of obtaining a cell which comprises the nucleic acid sequences which are used in the process and which code for an enzyme having threonine aldolase activity or lysine decarboxylase activity, where a cell is transformed with the nucleic acid sequences, with a gene construct (=nucleic acid construct) or with a vector, which bring about expression of the aldolase or decarboxylase nucleic acid on its own or in combination with other genes or sequences. In a further preferred embodiment, this process also includes the step of obtaining the amino acid(s) or the amino acid mixture from the culture and/or the organism. The cell prepared in this way is advantageously a cell of a plant described as advantageous as above, or of a microorganism.

Transgenic organism such as a plant or a transgenic microorganism means for the purposes of the invention that the nucleic acids used in the process are not at their natural site in the genome of an organism, and it is possible for the nucleic acids to be expressed homologously or heterologously. However, transgenic also means that the nucleic acids of the invention are in their natural place in the genome of an organism but that the sequence has been modified compared with the natural sequence and/or that the regulatory sequences of the natural sequences have been modified. Transgenic preferably means expression of the nucleic acids used in the process of the invention at an unnatural site in the genome, i.e. there is homologous or, preferably, heterologous expression of the nucleic acids. Expression may moreover take place transiently or from a sequence stably integrated in the genome. Preferred transgenic plants are, for example, the following plants selected from the Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, Iridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae families, preferably a plant selected from the group of families Apiaceae, Asteraceae, Brassicaceae, Cucurbitaceae, Fabaceae, Papaveraceae, Rosaceae, Solanaceae, Liliaceae or Poaceae. Further advantageous preferred plants are useful plants advantageously selected from the group of the genus of peanut, oilseed rape, canola, sunflower, safflower, olive, sesame, hazelnut, almond, avocado, bay, pumpkin, flax, soybean, pistachio, borage, corn, wheat, rye, oats, millet, triticale, rice, barley, cassaya, potato, sugar beet, aubergine, alfalfa and perennial grasses and feed crops, oil palm, vegetables (brassicas, roots, tubers, legumes, fruit vegetables, bulbs, leaf and stem vegetables), buckwheat, Jerusalem artichoke, broad bean, vetches, lentil, dwarf bean, lupin, clover and luceme.

The term “transgenic plant” used according to the invention also refers to the progeny of a transgenic plant, e.g. the T₁_{^{−}}, T₂_{^{−}}, T₃_{^{−}} and subsequent plant generations or the BC₁_{^{−}}, BC₂_{^{−}}, BC₃_{^{−}} and subsequent plant generations. Thus, the transgenic plants of the invention can be grown and crossed with themselves or other individuals in order to attain further transgenic plants of the invention. Transgenic plants can also be obtained by vegetative propagation of transgenic plant cells. The present invention also relates to transgenic plant material which can be derived from a population according to the invention of transgenic plants. This includes plant cells and certain tissues, organs and parts of plants in all their manifestations, such as seeds, leaves, anthers, fibers, roots, root hairs, stems, embryos, calli, cotyledons, petioles, harvest material, plant tissue, reproductive tissue and cell cultures, which is derived from the actual transgenic plant and/or can be used to produce the transgenic plant.

Transgenic plants containing the amino acids synthesized in the process of the invention can be marketed directly without isolating the synthesized compounds. Plants mean in the process of the invention all plant parts, plant organs such as leaf, stalk, root, tubers or seeds or the whole plant. The seed includes in this connection all seed parts such as the seed cases, epidermal and seed cells, endosperm or embryo tissue. The amino acids prepared in the process of the invention or the advantageously prepared amino acid L-methionine may, however, also be isolated from the plants in the form of their free amino acids or bound in proteins. Amino acids prepared by this process can be harvested by harvesting the organisms either from the culture in which they are growing, or from the field. This can take place by pressing, grinding and/or extraction, salt precipitation and/or ion exchange chromatography of the plant parts, preferably of the plant seeds, fruit, tubers, etc.

It is possible in this way to isolate more than 50% by weight, advantageously more than 60% by weight, preferably more than 70% by weight, particularly preferably more than 80% by weight, very particularly preferably more than 90% by weight, of the amino acids prepared in the process. The amino acids obtained in this way can then be further purified where appropriate, mixed if desired with other active ingredients such as vitamins, amino acids, carbohydrates, antibiotics, etc. and formulated where appropriate.

A further embodiment according to the invention is the use of the amino acids prepared in the process or of the transgenic organisms in animal or human foods, cosmetics or pharmaceuticals.

The nucleic acids used in the process can be integrated after introduction into a plant cell or plant either in the plastid genome or, preferably, in the genome of the host cell, and transient expression is possible and can be used advantageously. Production through, for example, viral infection with recombinant virus is also possible in principle, and in this case the expression of the gene or genes is advantageously increased. On integration into the genome, the integration may be random or take place via recombination such that the native gene is replaced by the introduced copy, thus modulating production of the desired compound by the cell, or by use of a gene in trans so that the gene is functionally connected to a functional expression unit which comprises at least one sequence ensuring expression of a gene and at least one sequence ensuring polyadenylation of a functionally transcribed gene. The nucleic acids are advantageously put into the plants via multiexpression cassettes or constructs for multiparallel expression of genes. In a further advantageous embodiment, the nucleic acid sequence is introduced in a simple expression cassette or a simple construct, i.e. without other different nucleic acid sequences, into the plant. Heterologous nucleic acid sequences are preferably introduced.

It is possible by using cloning vectors in plants and in the plant transformation such as those published and cited in: Plant Molecular Biology and Biotechnology (CRC Press, Boca Raton, Fla.), Chapter 617, pages 71-119 (1993); F. F. White, Vectors for Gene Transfer in Higher Plants; in: Transgenic Plants, Vol. 1, Engineering and Utilization, editors: Kung and R. Wu, Academic Press, 1993, 15-38; B. Jenes et al., Techniques for Gene Transfer, in: Transgenic Plants, Vol. 1, Engineering and Utilization, editors: Kung and R. Wu, Academic Press (1993),128-143; Potrykus, Annu. Rev. Plant Physiol. Plant Molec. Biol. 42 (1991), 205-225 to use the nucleic acids for genetic manipulation of a wide range of plants so that the latter becomes a better or more efficient producer of the amino acids prepared in the process of the invention. This improved production or efficiency of production of the amino acids or products derived therefrom, such as modified proteins, can be brought about by a direct effect of the manipulation or an indirect effect of this manipulation.

There is a number of mechanisms by which the modification of the threonine aldolase or lysine decarboxylase protein used in the process of the invention can directly influence the yield, production and/or efficiency of production of the amino acids from one of the transgenic plants or the microorganisms such as a yeast, a fungus or a bacterium on the basis of a modified protein. The number or activity of the threonine aldolase or lysine decarboxylase protein or gene can be increased so that this enzymic activity results in larger amounts of the desired product being prepared de novo because the organisms for example lacked the introduced enzymatic activity and thus the ability to increase the biosynthesis before introduction of the corresponding gene. However, expression of the gene naturally present in the organisms can also be increased, for example through a modified regulation of the gene, or the stability of the mRNA or of the gene product, i.e. of the aldolase or the decarboxylase, can be increased. Corresponding statements apply to the combination with other enzymes useful for synthesizing the amino acids from the biosynthesis metabolism. The use of various divergent sequences, i.e. ones which are different at the DNA sequence level, may also be advantageous in this connection, or the use of promoters for the gene expression which makes gene expression at a different time possible.

It is possible by introducing a threonine aldolase or lysine decarboxylase gene or a plurality of aldolase and/or decarboxylase genes into an organism alone or in combination with other genes not only to increase the biosynthetic flux to the final product but also to increase, alter or create de novo a product composition present in the organism. It is likewise possible to increase the number or activity of other genes in the import or export of nutrients of the cell(s) which are necessary for biosynthesis of the amino acids, so that the concentration of these precursors, cofactors or intermediates within the cell(s) or within the storage compartment is increased, thus further increasing the ability of the cells to produce amino acids, as described below. The yield, production and/or efficiency of production of amino acids in the host organism, such as the plants or the microorganisms, can be increased by optimizing the activity or increasing the number of threonine aldolase or lysine decarboxylase nucleic acid sequences and/or further genes involved in the biosynthesis of the amino acids, or by destroying the activity of one or more genes involved in the degradation of the amino acids.

Through this influencing of metabolism it is possible in the process of the invention to prepare further advantageous sulfur-containing compounds which comprise at least one covalently bonded sulfur atom. Examples of such compounds are besides methionine, homocysteine, S-adenosylmethionine, cysteine, advantageously methionine and S-adenosylmethionine.

The terms “L-methionine”, “methionine”, “homocysteine” and “S-adenosylmethionine” also include for the purposes of the present invention the corresponding salts such as, for example, methionine hydrochloride or methionine sulfate. The terms methionine or threonine are also intended to include the terms L-methionine or L-threonine. Also included are proteins in which the methionine prepared in the process are bound.

The isolated nucleic acid molecules used in the process of the invention code for proteins or parts thereof, where the proteins or the individual protein or parts thereof comprises an amino acid sequence which is sufficiently homologous with an amino acid sequence of the sequence SEQ ID NO: 2, SEQ ID NO: 12, SEQ ID NO: 14, SEQ ID NO: 16, SEQ ID NO: 18, SEQ ID NO: 20, SEQ ID NO: 22, SEQ ID NO: 24 or SEQ ID NO: 26 that the protein or the part thereof retains a threonine aldolase or lysine decarboxylase activity. The protein or the part thereof which is encoded by the nucleic acid molecule preferably has its essential enzymatic or biological activity and the ability to take part in the metabolism of amino acids in plants or microorganisms and generally in plant or microorganism metabolism or in the transport of molecules across membranes. The protein encoded by the nucleic acid molecules is advantageously at least about 30%, 35%, 40%, 45% or 50%, preferably at least about 60% and more preferably at least about 70%, 80% or 90% and most preferably at least about 95%, 96%, 97%, 98%, 99% or more homologous with an amino acid sequence of the sequence SEQ ID NO: 2. The protein is preferably a full-length protein which is substantially in parts homologous with a complete amino acid sequence of the SEQ ID NO: 2, SEQ ID NO: 12, SEQ ID NO: 14, SEQ ID NO: 16, SEQ ID NO: 18, SEQ ID NO: 20, SEQ ID NO: 22, SEQ ID NO: 24 or SEQ ID NO: 26 (which is derived from the open reading frame shown in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25). Further advantageous further nucleic acid sequences used in the process of the invention are derived from the amino acid sequences SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. The proteins encoded by these derived nucleic acid molecules are advantageously at least about 70% or 75%, preferably about at least 80% or 85%, more preferably at least about 90%, 91%, 92% or 94% and most preferably at least about 95%, 96%, 97%, 98%, 99% or more homologous with the amino acid sequences encoded by them or with an amino acid sequence of the sequence SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. Homology or homologous means for the purposes of the invention identity or identical.

Essential enzymatic or biological activity of the enzymes used means that, compared with the proteins/enzymes encoded by the sequences having SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 or the sequences derived from the sequences SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10, and derivatives thereof, they still have at least an enzymatic or biological activity of at least 10%, preferably 20%, particularly preferably 30% and very especially 40% and are thus able to take part in the metabolism of amino acids in compounds necessary for a plant or microorganism cell or in the transport of molecules across membranes, where the amino acids methionine or lysine are advantageously meant.

Nucleic acids which can be used in the process are advantageously derived from yeasts such as of the Saccharomycetaceae family such as the advantageous genus Saccharomyces or yeast genera such as Candida, Hansenula, Rhodotorula or Schizosaccharomyces and the particularly advantageous genus and species Saccharomyces cerevisiae. Its sequence is deposited under the EMBL accession numbers Z49330, Y13136 or YJL055W in the EMBL database as “hypothetical 26.9 kDa protein” or U18779, L10830 and U00092 in the EMBL database as product Gly1p (protein required for glycine prototrophy), CDS complement (14603.15766) with the protein ID=“AAB64996.1” and db_xref=“GI: 603634”.

An alternative possibility is to use in the process of the invention isolated nucleotide sequences which code for putative aldolases or decarboxylases and which hybridize onto a nucleotide sequence of SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 or, in another advantageous embodiment, onto a sequence derived from the sequences SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10, e.g hybridize under stringent conditions. The hybridization should advantageously be carried out with fragments of a length of at least 200 bp, advantageously at least 400 bp, preferably at least 600 bp, particularly preferably of at least 800 bp, very particularly preferably of at least 1000 bp. In a particularly preferred embodiment, the hybridization should be carried out with the complete nucleic acid sequence.

The nucleic acid sequences used in the process are advantageously introduced in an expression cassette (=nucleic acid construct) which makes expression of the nucleic acids possible in an organism, advantageously a plant or a microorganism.

For the introduction, the codogenic gene segment is advantageously subjected to an amplification and ligation in a known manner. The procedure is preferably based on the Pfu DNA polymerase protocol or a Pfu/Taq DNA polymerase mixture protocol. The primers are chosen on the basis of the sequence to be amplified. The primers should expediently be chosen so that the amplicon includes the complete codogenic sequence from start codon to stop codon. Following the amplification, the amplicon is expediently analyzed. The analysis can take place for example with regard to quality and quantity after fractionation by gel electrophoresis. The amplicon can then be purified in accordance with a standard protocol (e.g. Qiagen). An aliquot of the purified amplicon is then available for subsequent cloning. Suitable cloning vectors are generally known to the skilled worker. These include in particular vectors which are able to replicate in bacterial systems, i.e. especially vectors which ensure efficient cloning in E. coli, and which make stable transformation of plants possible. Mention should be made in particular of various binary and cointegrated vector systems suitable for T-DNA-mediated transformation. Vector systems of this type are usually characterized by comprising at least the vir genes necessary for agrobacterium-mediated transformation, and the T-DNA border sequences. These vector systems preferably also comprise further cis-regulatory regions such as promoters and terminators and/or selection markers with which appropriately transformed organisms can be identified. Whereas vir genes and T-DNA sequences are arranged on the same vector in cointegrated vector systems, binary systems are based on at least two vectors, one of which harbors a vir gene but no T-DNA, and a second harbors T-DNA but no vir gene. This makes the latter vectors relatively small, easy to manipulate and easy to replicate both in E. coli and in Agrobacterium. These binary vectors include vectors of the pBIB-HYG, pPZP, pBecks, pGreen series. Preferably used according to the invention are Bin19, pBI101, pBinAR, pGPTV and pCAMBIA. A review of binary vectors and their use is given by Hellens et al, Trends in Plant Science (2000) 5, 446-451. For vector preparation, the vectors can be initially linearized with restriction endonuclease(s) and then enzymatically modified in a suitable way. The vector is subsequently purified, and an aliquot is employed for the cloning. In the cloning, the enzymatically cut and, if necessary, purified amplicon is cloned with similarly prepared vector fragments using ligase. It is moreover possible for a particular nucleic acid construct or vector or plasmid construct to have one or else more than one codogenic gene segments. The codogenic gene segments in these constructs are preferably functionally linked to regulatory sequences. The regulatory sequences include in particular plant sequences such as the promoters and terminators described above. The constructs are advantageously capable of stable propagation in microorganisms, especially Escherichia coli and Agrobacterium tumefaciens, under selective conditions, and make transfer of heterologous DNA possible into plants or other microorganisms. In a particular embodiment, the constructs are based on binary vectors (review of binary vectors in Hellens et al., 2000). The latter usually comprise prokaryotic regulatory sequences such as origin of replication and selection markers for replication in microorganisms such as Escherichia coli and Agrobacterium tumefaciens, and agrobacterium T-DNA sequences for the purpose of transferring DNA into plant genomes. Of the complete Agrobacterium T-DNA sequence, at least the right border sequence comprising about 25 base pairs is required. The vector constructs of the invention usually comprise T-DNA sequences both from the right and from the left border region, which expediently comprise recognition sites for enzymes which act site-specifically and which in turn are encoded by part of the vir genes. Suitable host organisms are known to the skilled worker. Advantageous organisms are described above in this application. These include in particular bacterial hosts, of which some have already been mentioned above in connection with donor microorganisms, e.g. microorganisms such as fungi such as the genus Claviceps or Aspergillus or Gram-positive bacteria such as the genera Bacillus, Corynebacterium, Micrococcus, Brevibacterium, Rhodococcus, Nocardia, Caseobacter or Arthrobacter or Gram-negative bacteria such as the genera Escherichia, Flavobacterium or Salmonella or yeasts such as the genera Rhodotorula, Hansenula or Candida. Particularly advantageous organisms are selected from the group of genera Corynebacterium, Brevibacterium, Escherichia, Bacillus, Rhodotorula, Hansenula, Candida, Claviceps or Flavobacterium. It is very particularly advantageous to use in the process of the invention microorganisms selected from the group of genera and species consisting of Hansenula anomala, Candida ublis, Claviceps purpurea, Bacillus circulans, Bacillus subtilis, Bacillus sp., Brevibacterium albidum, Brevibacterium album, Brevibacterium cerinum, Brevibacterium flavum, Brevibacterium glutamigenes, Brevibacterium iodinum, Brevibacterium ketoglutamicum, Brevibacterium lactofermentum, Brevibacterium linens, Brevibacterium roseum, Brevibacterium saccharolyticum, Brevibacterium sp., Corynebacterium acetoacidophilum, Corynebacterium acetoglutamicum, Corynebacterium ammoniagenes, Corynebacterium glutamicum (=Micrococcus glutamicum), Corynebacterium melassecola, Corynebacterium sp. or Escherichia coli, specifically Escherichia coli K12 and its described strains. Advantageously preferred according to the invention are host organisms of the genus Escherichia, in particular Escherichia coli, and Agrobacterium, in particular Agrobacterium tumefaciens, or plants selected from the Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, Iridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae families, preferably a plant selected from the group of families Apiaceae, Asteraceae, Brassicaceae, Cucurbitaceae, Fabaceae, Papaveraceae, Rosaceae, Solanaceae, Liliaceae or Poaceae. Further advantageous preferred plants are useful plants advantageously selected from the group of the genus of peanut, oilseed rape, canola, sunflower, safflower, olive, sesame, hazelnut, almond, avocado, bay, pumpkin, flax, soybean, pistachio, borage, corn, wheat, rye, oats, millet, triticale, rice, barley, cassaya, potato, sugar beet, feed beet, aubergine and perennial grasses and feed crops, oil palm, vegetables (brassicas, roots, tubers, legumes, fruit vegetables, bulbs, leaf and stem vegetables), buckwheat, Jerusalem artichoke, broad bean, vetches, lentil, alfalfa, dwarf bean, lupin, clover and luceme. For introducing the nucleic acids used in the process of the invention into a plant it has proved to be advantageous initially to transfer them into an intermediate host, e.g. a bacterium. Transformation into E. coli has proved expedient in this connection and can be carried out in a manner known per se, e.g. by heat shock or electroporabon. Thus, the transformed E. coli colonies can be investigated for the cloning efficiency. This can take place with the aid of a PCR. It is moreover possible to examine both the identity and the integrity of the plasmid construct on the basis of a defined number of colonies by subjecting an aliquot of the colonies to said PCR. The primers employed for this purpose are generally universal primers derived from vector sequences, with the forward primer being disposed upstream of the start ATG and the reverse primer being disposed downstream of the stop codon of the codogenic gene segment. The amplicons are fractionated by electrophoresis and assessed for quantity and quality. Detection of a fragment of the appropriate size leads to a positive assessment. The plasmid constructs which are examined where appropriate are subsequently used for transforming the plants. It may for this purpose initially be necessary to obtain the constructs from the intermediate host. The constructs can, for example, be obtained as plasmids from bacterial hosts on the basis of a conventional plasmid isolation. Numerous processes for transforming plants are known. Since stable integration of heterologous DNA into the genome of plants is advantageous according to the invention, T-DNA-mediated transformation has proved to be particularly expedient. It is for this purpose initially necessary to transform suitable vehicles, especially agrobacteria, with the codogenic gene segment or the corresponding plasmid construct. This can take place in a manner known per se. For example, the plasmid construct produced in accordance with the above statements can be transformed by means of electroporation or heat shock into competent agrobacteria. A distinction must be made in this connection in principle between the formation of cointegrated vectors on the one hand and transformation with binary vectors. In the first alternative, the vector constructs including the codogenic gene segment have no T-DNA sequences; on the contrary, the formation of the cointegrated vectors takes place in the agrobacteria through homologous recombination of the vector construct with T-DNA. The T-DNA is present in the agrobacteria in the form of Ti or Ri plasmids in which the oncogenes have expediently been replaced by exogenous DNA On use of binary vectors it is possible to transfer them by bacterial conjugation or direct transfer to agrobacteria. These agrobacteria expediently already contain the vector which harbors the vir genes (frequently referred to as helper Ti(Ri) plasmid). Together with the plasmid construct and T-DNA it is expediently possible also to use one or more markers using which it is possible to select transformed agrobacteria and transformed plant cells. A large number of markers has been developed for this purpose. These include, for example, those conferring resistance to chloramphenicol, kanamycin, the aminoglycoside G418, hygromycin and the like. It is usually desired for the plasmid constructs to be flanked on one or both sides of the codogenic gene segment by T-DNA This is particularly useful when bacteria of the gene species Agrobacterium tumefaciens or Agrobacterium rhizogenes are used for the transformation. A method preferred according to the invention is transformation using Agrobacterium tumefaciens. However, biolistic methods can also be used advantageously for inserting the sequences in the process of the invention, and insertion using PEG is also possible. The transformed agrobacteria can be cultured in a manner known per se and are thus available for expedient transformation of the plants. The plants or plant parts to be transformed are grown or provided in a conventional way. The plants or plant parts are then exposed to the transformed agrobacteria until an adequate transformation rate is reached. The plants and plant parts can be exposed to agrobacteria in various ways. For example, a culture of morphogenic plant cells or tissues can be used. Following the T-DNA transfer, the bacteria are usually eliminated by antibiotics, and the regeneration of plant tissue is induced. Suitable plant hormones are used in particular for this purpose in order, after initial callus formation, to promote the formation of shoots. An advantageous transformation method is in planta transformation. For this purpose, it is possible to expose plant seeds for example to the agrobacteria, or to inoculate plant meristem with agrobacteria. It has proved particularly expedient according to the invention to expose the whole plant or at least the flower primordia to a suspension of transformed agrobacteria. The former is then grown further until seeds of the treated plant are obtained (Clough and Bent, Plant J. (1998) 16, 735-743). To select transformed plants, the plant material obtained from the transformation is usually subjected to selective conditions so that transformed plants can be distinguished from untransformed plants. For example, the seeds obtained in the manner described above can be sown anew and, after growing, subjected to a suitable spray selection. A further possibility is to grow the seeds, if necessary after sterilization, on agar plates using a suitable selecting agent in such a way that only the transformed seeds are able to grow to plants. Further advantageous transformation methods in particular of plants are known to the skilled worker and are described below.

The nucleic acid sequences coding for the threonine aldolase and/or lysine decarboxylase used in the process of the invention are functionally linked to one or more regulatory signals, advantageously for increasing gene expression, in the process of the invention. These regulatory sequences are intended to make specific expression of the genes and protein expression possible. This may mean, for example, depending on the host organism (=transgenic organism, e.g. plant or microorganism), that the gene is expressed and/or overexpressed only after induction, or that it is immediately expressed and/or overexpressed. Examples of these regulatory sequences are sequences to which inducers or repressors bind and thus regulate the expression of the nucleic acid. In addition to these new regulatory sequences or in place of these sequences it is possible for the natural regulation of these sequences still to be present in front of the actual structural genes and, where appropriate, to have been genetically modified so that the natural regulation has been switched off and the expression of the genes has been increased. The expression cassette (=expression construct=gene construct=nucleic acid construct) may, however, also have a simpler structure, i.e. no additional regulatory signals have been inserted in front of the nucleic acid sequence or its derivatives, and the natural promoter with its regulation has not been deleted. Instead, the natural regulatory sequence has been mutated so that regulation no longer takes place and/or gene expression is increased. These modified promoters can also be put in the form of partial sequences (=promoter with parts of the nucleic acid sequences of the invention) alone in front of the natural gene to increase the activity. The gene construct may additionally advantageously also comprise one or more so-called “enhancer sequences” functionally linked to the promoter, which make increased expression of the nucleic acid sequence possible. Additional advantageous sequences can also be inserted at the 3′ end of the DNA sequences, such as further regulatory elements or terminators. The nucleic acid sequence(s) coding for the threonine aldolase proteins may be present in one or more copies in the expression cassette (=nucleic acid construct). It is advantageous for only one copy in each case of the genes to be present in the expression cassette. This nucleic acid construct or the nucleic acid constructs may be expressed together in the host organism. It is moreover possible for the nucleic acid construct or the nucleic acid constructs to be inserted, advantageously, in one or more vectors and be present free in the cell, or else be inserted in the genome. In the case of plants, integration into the plastid genome or, preferably, into the cell genome can take place. It is advantageous for insertion of further genes in the host genome if the genes to be expressed are present together in one gene construct.

Regulatory sequences are usually disposed upstream (5′), within and/or downstream (3′) in relation to a particular nucleic acid or a particular codogenic gene segment. They control in particular the transcription and/or translation, and the transcript stability of the codogenic gene segment, where appropriate in cooperation with further functional systems intrinsic to the cell, such as the protein biosynthesis apparatus of the cell.

Regulatory sequences include in particular sequences disposed upstream (5′), which relate in particular to regulation of transcription initiation, such as promoters, and sequences disposed downstream (3′), which relate in particular to regulation of transcription termination, such as polyadenylation signals.

Promoters which can be employed are in principle all those able to stimulate transcription of genes in organisms such as microorganisms, plants or nonhuman animals. Suitable promoters able to function in these organisms are generally known. They may be constitutive or inducible promoters. Suitable promoters may in multicellular eukaryotes make development- and/or tissue-specific expression possible, and it is thus possible in plants advantageously to use leaf-, root-, flower-, seed-, guard cell- or fruit-specific promoters.

The regulatory sequences or factors may moreover, as described above, preferably have a positive influence, and thus increase, gene expression of the introduced genes. Thus, the regulatory elements can advantageously be strengthened at the level of transcription by using strong transcription signals such as promoters and/or enhancers. Besides this, however, it is also possible to enhance translation by, for example, introducing translation enhancer sequences or improving the stability of the mRNA.

One or more nucleic acid constructs comprising one or more nucleic acid sequences which are defined by SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 and code for the polypeptides represented in SEQ ID NO: 2, SEQ ID NO: 12, SEQ ID NO: 14, SEQ ID NO: 16, SEQ ID NO: 18, SEQ ID NO: 20, SEQ ID NO: 22, SEQ ID NO: 24 or SEQ ID NO: 26 are a further embodiment of the invention. One or more nucleic acid constructs comprising one or more nucleic acid sequences which can be derived from the sequences of the invention SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 are a further advantageous embodiment of the invention. Said polypeptides advantageously have threonine aldolase activity. The same applies to their homologs, derivatives or analogs which are functionally connected to one or more regulatory signals, advantageously to increase gene expression.

Advantageous regulatory sequences for the novel process are present for example in promoters such as the cos, tac, rha, trp, tet, trp-tet, lpp, lac, lpp-lac, lacl^{q-}, T7, T5, T3, gal, trc, ara, SP6, λ-P_{R} or λ-P_{L} promoter, which are advantageously used in Gram-negative bacteria. Further advantageous regulatory sequences are present for example in the Gram-positive promoters amy, dnaK, xylS and SPO₂, in the yeast or fungus promoters ADC1, MF□, AC, P-60, UASH, MCB, PHO, CYC1, GAPDH, TEF, rp28, ADH or in the plant promoters CaMV/35S [Franck et al., Cell 21 (1980) 285-294, U.S. Pat. No. 5,352,605], PRP1 [Ward et al., Plant. Mol. Biol. 22 (1993)], SSU, PGEL1, OCS [Leisner and Gelvin (1988) Proc Natl Acad Sci USA 85(5):2553-2557], lib4, usp, mas [Comai et al. (1990) Plant Mol Biol 15 (3):373-381], STLS1, ScBV Schenk et al. (1999) Plant Mol Biol 39(6):1221-1230, B33, SAD1 or SAD2 (Flachspromotoren, Jain et al., Crop Science, 39 (6), 1999: 1696-1701) or nos [Shaw et al. (1984) Nucleic Acids Res. 12(20):7831-7846]. It is also possible and advantageous to use the various ubiquitin promoters from Arabidopsis [Callis et al. (1990) J. Biol. Chem., 265:12486-12493; Holtorf S et al. (1995) Plant. Mol. Biol., 29:637-747], Pinus, corn [(Ubi1 und Ubi2), U.S. Pat. No. 5,510,474; U.S. Pat. No. 6,020,190 and U.S. Pat. No. 6,054,574] or parsley [Kawalleck et al., Plant Molecular Biology, 21, 1993: 673-684] or phaseolin promoter. Likewise advantageous in this connection are inducible promoters such as the promoters described in EP-A-0 388 186 (benzylsulfonamide-inducible), Plant J. 2, 1992:397-404 (Gatz et al., tetracycline-inducible), EP-A-0 335 528 (abscisic acid-inducible) or WO 93/21334 (ethanol- or cyclohexenol-inducible). Further suitable plant promoters are the promoter of cytosilic FBPase or the potato ST-LSI promoter (Stockhaus et al., EMBO J. 8, 1989, 2445), Glycine max phosphoribosyl-pyrophosphate amidotransferase promoter (Genbank access No. U87999) or the node-specific promoter described in EP-A-0 249 676. Particularly advantageous promoters are promoters which make expression possible in specific tissues or show a preferential expression in certain tissues. Also advantageous are seed-specific promoters such as the USP promoter of the embodiment, but also other promoters such as the LeB4, DC3, SAD1, phaseolin or napin promoter. Further particularly advantageous promoters are seed-specific promoters which can be used for monocotyledonous or dicotyledonous plants and are described in U.S. Pat. No. 5,608,152 (oilseed rape napin promoter), WO 98/45461 (Arabidopsis oleosin promoter), U.S. Pat. No. 5,504,200 (Phaseolus vulgaris phaseolin promoter), WO 91/13980 (brassica Bce4 promoter), and by Baeumlein et al., Plant J., 2, 2, 1992:233-239 (legume LeB4 promoter), these promoters being suitable for dicotyledons. The following promoters are suitable for example for monocotyledons barley Ipt-2 or Ipt-1 promoter (WO 95/15389 and WO 95/23230), barley hordein promoter, the corn ubiquitin promoter and other suitable promoters described in WO 99/16890.

It is possible in principle to use all natural promoters with their regulatory sequences, such as the abovementioned, for the novel process. It is likewise possible and advantageous to use synthetic promoters additionally or alone, especially if they mediate seed-specific expression as described, for example, in WO 99/16890.

In order to achieve a particularly effective content of threonine aldolase and/or lysine decarboxylase proteins in transgenic plants, the encoded biosynthesis genes can advantageously be expressed constitutively and/or seed-, fruit- or tuber-specifically in plants. In a further advantageous embodiment, however, they may also be inducibly expressed, so that they are induced, and thus expressed, specifically in a desired growth phase of the plant. It is possible to use for this purpose seed-specific promoters or promoters which are active in the embryo and/or in the endosperm. Seed-specific promoters can in principle be isolated both from dicotyledonous and from monocotyledonous plants. Advantageous preferred promoters are listed in the following: USP (=unknown seed protein) and vicilin (Vicia faba) [Bäumlein et al., Mol. Gen Genet., 1991, 225(3)], napin (oilseed rape) [U.S. Pat. No. 5,608,152], acyl carrier protein (oilseed rape) [U.S. Pat. No. 5,315,001 and WO 92/18634], oleosin (Arabidopsis thaliana) [WO 98/45461 and WO 93/20216], phaseolin (Phaseolus vulgaris) [U.S. Pat. No. 5,504,200], Bce4 [WO 91/13980], legume B4 (LegB4 promoter) [Baumlein et al., Plant J., 2, 2, 1992], Lpt2 and Ipt1 (barley) [WO 95/15389 and WO95/23230], seed-specific promoters from rice, corn and wheat [WO 99/16890], Amy32b, Amy 6-6 and aleurain [U.S. Pat. No. 5,677,474], Bce4 (oilseed rape) [U.S. Pat. No. 5,530,149], glycinin (soybean) [EP 571 741], phosphoenolpyruvate carboxylase (soybean) [JP 06/62870], ADR12-2 (soybean) [WO 98/08962], isocitrate lyase (oilseed rape) [U.S. Pat. No. 5,689,040] or Omylase (barley) [EP 781 849].

Plant gene expression can also be facilitated by a chemically inducible promoter (see a review in Gatz 1997, Annu. Rev. Plant Physiol. Plant Mol. Biol., 48:89-108). Chemically inducible promoters are particularly suitable when it is desired for gene expression to take place in a time-specific manner. Examples of such promoters are a salicylic acid-inducible promoter (WO 95/19443), tetracycline-inducible promoter (Gatz et al. (1992) Plant J. 2, 397-404) and ethanol-inducible promoter.

Expression specifically in gymnosperms or angiosperms is also possible in principle.

In order to ensure stable integration of nucleic acid sequences used in the process of the invention in combination with further biosynthesis genes in the transgenic plant over several generations, each of the nucleic acids which are used in the process and code for the aldolases and/or decarboxylases should be expressed under the control of its own, preferably of a different, promoter, because repeating sequence motifs may lead to instability of the T-DNA or to recombination events or to silencing. The structure of the expression cassette is advantageously such that a promoter is followed by a suitable cleavage site for inserting the nucleic acid to be expressed, advantageously in a polylinker subsequently where appropriate a terminator is located behind the polylinker. This successive arrangement is repeated a plurality of times, preferably three, four or five times, so that up to five genes can be combined in a construct and thus be introduced for expression into the transgenic plant. The successive arrangement is advantageously repeated up to three times. The nucleic acid sequences are inserted for expression via the suitable cleavage site, for example in the polylinker behind the promoter. It is advantageous for each nucleic acid sequence to have its own promoter and, where appropriate, its own terminator. However, it is also possible for a plurality of nucleic acid sequences to be inserted behind a promoter and, where appropriate, in front of a terminator. The insertion site or the successive arrangement of the inserted nucleic acids in the expression cassette is not of crucial importance, which means that a nucleic acid sequence can be inserted in first or last place in the cassette with the expression being negligibly influenced thereby. It is possible and advantageous to use in the expression cassette different promoters such as, for example, the USP, the LegB4, the DC3 promoter or the ubiquitin promoter from parsley and different terminators. It is, however, also possible to use only one type of promoter in the cassette. This may, however, lead to unwanted recombination events or silencing effects. A further advantageous nucleic acid sequence which can be expressed in combination with the sequences used in the process and/or the aforementioned biosynthesis genes is the sequence for an ATP/ADP translocator as described in WO 01/20009. This ATP/ADP translocator leads to an increase in the synthesis of the essential amino acids lysine and/or methionine, advantageously methionine.

As described above, the transcription of the introduced genes should advantageously be stopped by suitable terminators at the 3′ end of the introduced biosynthesis genes (behind the stop codon). It is possible to use for this purpose, for example, the OCS1 terminator. Just as for the promoters, different terminator sequences should be used for each gene here.

The gene construct may, as described above, also include other genes which are to be introduced into the organisms. It is possible and advantageous for regulatory genes, such as genes for inducers, repressors or enzymes, which intervene through their enzymic activity in the regulation of one or more genes of a biosynthetic pathway, to be introduced into the host organisms and to be expressed therein. These genes may be of heterologous or homologous origin. The nucleic acid construct or gene construct may also advantageously contain further biosynthesis genes, or else these genes may be located on another or a plurality of other nucleic acid constructs. Biosynthesis genes advantageously used are genes of amino acid metabolism, of glycolysis, of tricarboxylic acid metabolism or combinations thereof.

It is moreover possible for the aforementioned polypeptides or enzymes to be cloned in combination with further genes in the nucleic acid constructs or vectors and be employed for transforming microorganisms or plants with the aid of, for example, Agrobacterium.

The regulatory sequences or factors may moreover, as described above, preferably have a positive influence, and thus increase, gene expression of the introduced genes. Thus, the regulatory elements can advantageously be strengthened at the level of transcription by using strong transcription signals such as promoters and/or enhancers. Besides this, however, it is also possible to enhance translation by, for example, introducing translation enhancer sequences or improving the stability of the mRNA The expression cassettes can in principle be used directly for introduction into the plant, or else be introduced into a vector.

These advantageous vectors, preferably expression vectors, comprise the nucleic acid which are used in the process and which code for threonine aldolase and/or lysine decarboxylase proteins, or a nucleic acid construct which comprises the nucleic acid used, alone or in combination with further genes such as the biosynthesis genes of amino acid metabolism. The term “vector”, as used herein, relates to a nucleic acid molecule which is able to transport another nucleic acid to which it is linked. One type of vector is a “plasmid” which stands for a circular double-stranded DNA loop into which additional DNA segments can be ligated. A further type of vector is a viral vector, in which case additional DNA segments can be ligated into the viral genome. Certain vectors are capable of autonomous replication in a host cell into which they have been introduced (e.g. bacterial vectors with bacterial origin of replication). Other preferred vectors are advantageously integrated on introduction into the host cell into the genome of a host cell and thus replicated together with the host genome. In addition, certain vectors are able to control the expression of genes to which they are functionally connected. These vectors are referred to here as “expression vectors. As mentioned above, they are capable of autonomous replication or may be integrated into the host genome. Expression vectors suitable for DNA recombination techniques are usually in the form of plasmids. “Plasmid” and “vector” can be used exchangeably in the present description because the plasmid is the most commonly used vector form. However, the invention is intended to encompass these other expression vector forms such as viral vectors, which exercise similar functions. The term vector is also intended to encompass other vectors known to the skilled worker, such as phages, viruses such as SV40, CMV, TMV, transposons, IS elements, phasmids, phagemids, cosmids, linear or circular DNA.

The recombinant expression vectors advantageously used in the process include the nucleic acids of the invention or the nucleic acid construct of the invention in a form suitable for expression of the nucleic acids used in a host cell, meaning that the recombinant expression vectors include one or more regulatory sequences selected on the basis of the host cells to be used for the expression, which is functionally connected to the nucleic acid sequence to be expressed. In a recombinant expression vector, “functionally connected” means that the nucleotide sequence of interest is linked to the regulatory sequence(s) in such a way that expression of the nucleotide sequence is possible and they are linked to one another so that both sequences comply with the predicted function ascribed to the sequence (e.g. in an in vitro transcription/translation system or in a host cell when the vector is introduced into the host cell). The term “regulatory sequence” is intended to include promoters, enhancers and other expression control elements (e.g. polyadenylation signals). These regulatory sequences are described, for example, in Goeddel: Gene Expression Technology: Methods in Enzymology 185, Academic Press, San Diego, Calif. (1990), or see: Gruber and Crosby, in: Methods in Plant Molecular Biology and Biotechnolgy, CRC Press, Boca Raton, Fla., editors: Glick and Thompson, Chapter 7, 89-108, including the references therein. Regulatory sequences include those which control constitutive expression of a nucleotide sequence in many types of host cell, and those which control direct expression of the nucleotide sequence only in particular host cells under particular conditions. The skilled worker is aware that the design of the expression vector may depend on factors such as the choice of host cell to be transformed, the extent of expression of the desired protein etc.

The recombinant expression vectors used may be designed specifically for the expression of nucleic acid sequences used in the process in prokaryotic or eukaryotic cells. This is advantageous because intermediate steps of vector construction are often carried out for simplicity in microorganisms. For example, the amino acid genes, lysine decarboxylase genes and/or threonine aldolase genes can be expressed in bacterial cells, insect cells (using baculovirus expression vectors), yeast and other fungus cells [see Romanos, M. A, et al. (1992)° Foreign gene expression in yeast a review”, Yeast 8:423-488; van den Hondel, C. M. J. J., et al. (1991) “Heterologous gene expression in filamentous fungi”, in: More Gene Manipulations in Fungi, J. W. Bennet & L. L. Lasure, editors, pp. 396-428: Academic Press: San Diego; and van den Hondel, C. A. M. J. J., & Punt, P. J. (1991) “Gene transfer systems and vector development for filamentous fungi, in: Applied Molecular Genetics of Fungi, Peberdy, J. F., et al., editor, pp. 1-28, Cambridge University Press: Cambridge], algae [Falciatore et al., 1999, Marine Biotechnology.1, 3:239-251] with vectors in a transformation process as described in WO 98/01572, and preferably in cells of multicellular plants [see Schmidt, R. and Willmitzer, L. (1988) “High efficiency Agrobacterium tumefaciens-mediated transformation of Arabidopsis thaliana leaf and cotyledon explants” Plant Cell Rep.:583-586; Plant Molecular Biology and Biotechnology, C Press, Boca Raton, Fla., Chapter 617, pp. 71-119 (1993); F. F. White, B. Jenes et al., Techniques for Gene Transfer, in: Transgenic Plants, Vol. 1, Engineering and Utilization, editors: Kung and R. Wu, Academic Press (1993), 12843; Potrykus, Annu. Rev. Plant Physiol. Plant Molec. Biol. 42 (1991), 205-225 (and references cited therein)]. Suitable host cells are also discussed in Goeddel, Gene Expression Technology: Methods in Enzymology 185, Academic Press, San Diego, Calif. (1990). The sequence of the recombinant expression vector may alternatively be transcribed and translated in vitro, for example using T7 promoter regulatory sequences and T7 polymerase.

Expression of proteins in prokaryotes usually takes place with vectors containing constitutive or inducible promoters which control the expression of fusion or nonfusion proteins. Typical fusion expression vectors are, inter alia, pGEX (Pharmacia Biotech Inc; Smith, D. B., and Johnson, K. S. (1988) Gene 67:31-40), pMAL (New England Biolabs, Beverly, Mass.) and pRIT5 (Pharmacia, Piscataway, N.J.), in which glutathione S-transferase (GST), maltose E-binding protein and protein A, respectively, are fused to the recombinant target protein.

Examples of suitable inducible nonfusion E. coli expression vectors are, inter alia, pTrc (Amann et al. (1988) Gene 69:301-315) and pET 11d [Studier et al., Gene Expression Technology: Methods in Enzymology 185, Academic Press, San Diego, Calif. (1990) 60-89]. Target gene expression from the pTrc vector is based on transcription by host RNA polymerase from a hybrid trp-lac fusion promoter. Target gene expression from the pET 11d vector is based on transcription from a T7-gn10-lac fusion promoter which is mediated by a coexpressed viral RNA polymerase (T7 gn1). This viral polymerase is provided by the host strains BL21 (DE3) or HMS174 (DE3) by a resident α-prophage which harbors a T7 gn1 gene under the transcriptional control of the lacUV 5 promoter.

Other vectors suitable in prokaryotic organisms are known to the skilled worker, these vectors being, for example, in E. coli pLG338, pACYC184, the pBR series, such as pBR322, the pUC series such as pUC18 or pUC19, the M113 mp series, pKC30, pRep4, pHS1, pHS2, pPLc236, pMBL24, pLG200, pUR290, pIN-III¹¹³-B1, λgt11 or pBdCl, in Streptomyces pIJ101, pIJ364, pIJ702 or pIJ361, in Bacillus pUB110, pCl 94 or pBD214, in Corynebacterium pSA77 or pAJ667.

In a further embodiment, the expression vector is a yeast expression vector. Examples of vectors for expression in the yeast S. cerevisiae include pYe desaturase c1 (Baldari et al. (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.). Vectors and processes for constructing vectors suitable for use in other fungi such as the filamentous fungi include those described in detail in: van den Hondel, C. A. M. J. J., & Punt, P. J. [(1991) “Gene transfer systems and vector development for filamentous fungi, in: Applied Molecular Genetics of fungi, J. F. Peberdy et al., editors, pp. 1-28, Cambridge University Press: Cambridge; or in: More Gene Manipulations in Fungi; J. W. Bennet & L. L. Lasure, editors, pp. 396-428: Academic Press: San Diego]. Further suitable yeast vectors are, for example, 2□M, pAG-1, YEp6, YEp13 or pEMBLYe23.

Further vectors which may be mentioned by way of example are pALS1, pIL2 or pBB116 in fungi or pLGV23, pGHlac⁺, pBIN19, pAK2004 or pDH51 in plants.

An alternative possibility is to express the nucleic acid sequences in insect cells using baculovirus expression vectors. Baculovirus vectors available for expression of proteins in cultured insect cells (e.g. Sf9 cells) include the pAc series (Smith et al. (1983) Mol. Cell Biol. 3:2156-2165) and the pVL series (Lucklow and Summers (1989) Virology 170:31-39).

The abovementioned vectors provide only a small review of possible suitable vectors. Further plasmids are known to the skilled worker and are described for example in: Cloning Vectors (editors Pouwels, P. H., et al., Elsevier, Amsterdam-New York-Oxford, 1985, ISBN 0 444 904018). For further suitable expression systems for prokaryotic and eukaryotic cells, see Chapters 16 and 17 of Sambrook, J., Fritsch, E. F., and Maniatis, T., Molecular Cloning: A Laboratory Manual, 2nd edition, Cold Spring Harbor Laboratory, Cold Spring Harbor Laboratory Press, Cold Spring Harbor, N.Y., 1989.

In a further advantageous embodiment of the process, the nucleic acid sequences can be expressed in unicellular plant cells (such as algae), see Falciatore et al., 1999, Marine Biotechnology 1 (3):239-251 and references cited therein, and plant cells from higher plants (e.g. spermatophytes such as crops). Examples of plant expression vectors include those described in detail in: Becker, D., Kemper, E., Schell, J., and Masterson, R. [(1992) “New plant binary vectors with selectable markers located proximal to the left border, Plant Mol. Biol. 20:1195-1197] and Bevan, M. W. [(1984) “Binary Agrobacterium vectors for plant transformation”, Nucl. Acids Res. 12:8711-8721; Vectors for Gene Transfer in Higher Plants; in: Transgenic Plants, Vol. 1, Engineering and Utilization, editors: Kung and R. Wu, Academic Press, 1993, pp. 15-38]. A review of binary vectors and their use is also to be found in Hellens, R., Mullineaux, P. and Klee H., (2000) “A guide to Agrobacterium binary vectors, Trends in Plant Science, Vol. 5 No. 10, 446-451.

A plant expression cassette preferably comprises regulatory sequences able to control gene expression in plant cells and functionally connected so that each sequence is able to comply with its function, such as termination and transcription, for example polyadenylation signals. Preferred polyadenylation signals are those derived from Agrobacterium tumefaciens T-DNA, such as the gene 3 known as octopine synthase of the Ti plasmid pTiACH5 (Gielen et al., EMBO J. 3 (1984) 835ff.) or functional equivalents thereof, but all other terminators functionally active in plants are also suitable.

Since plant gene expression is very often not restricted at the levels of transcription, a plant expression cassette preferably comprises other functionally connected sequences such as translation enhancers, for example the overdrive sequence which comprises the 5′-untranslated leader sequence from tobacco mosaic virus which increases the protein/RNA ratio (Gallie et al., 1987, Nucl. Acids Research 15:8693-8711).

For expression in plants, the nucleic acid sequences must, as described above, be functionally connected to a suitable promoter which carries out gene expression in a timely, cell- or tissue-specific manner. Promoters which can be used are constitutive promoters (Benfey et al., EMBO J. 8 (1989) 2195-2202), such as those derived from plant viruses such as ³⁵S CAMV (Franck et al., Cell 21 (1980) 285-294), 19S CaMV (see also U.S. Pat. No. 5,352,605 and WO 84/02913), 34S FMV (Sanger et al., Plant. Mol. Biol., 14, 1990: 433-443), the parsley ubiquitin promoter or plant promoters such as that described in U.S. Pat. No. 4,962,028 of the rubisco small subunit.

Other preferred sequences for use for functional connection in plant gene expression cassettes are targeting sequences which are necessary for guiding the gene product into its appropriate cell compartment (see a review in Kermode, Crit. Rev. Plant Sci. 15, 4 (1996) 285-423 and references cited therein), for example into the vacuoles, the cell nucleus, all types of plastids such as amyloplasts, chloroplasts, chromoplasts, the extracellular space, the mitochondria, the endoplasmic reticulum, elaioplast, peroxisomes and other compartments of plant cells.

Plant gene expression can also be facilitated as described above by a chemically inducible promoter (see a review in Gatz 1997, Annu. Rev. Plant Physiol. Plant Mol. Biol., 48:89-108). Chemically inducible promoters are particularly suitable when time-specific gene expression is desired. Examples of such promoters are a salicylic acid-inducible promoter (WO 95/19443), a tetracycline-inducible promoter (Gatz et al. (1992) Plant J. 2, 397-404) and an ethanol-inducible promoter.

Promoters which respond to biotic or abiotic stress conditions are also suitable promoters, for example the pathogen-induced PRP1 gene promoter (Ward et al., Plant Mol. Biol. 22 (1993) 361-366), the heat-inducible tomato hsp80 promoter (U.S. Pat. No. 5,187,267), the cold-inducible potato alpha-amylase promoter (WO 96/12814) or the pinII promoter which is inducible by wounding (EP-A-0 375 091).

Particularly preferred promoters are those which bring about gene expression in tissues and organs in which amino acid biosynthesis takes place, in seed cells such as the cells of the endosperm and of the developing embryo. Suitable promoters are the oilseed rape napin gene promoter (U.S. Pat. No. 5,608,152), the Vicia faba USP promoter (Baeumlein et al., Mol Gen Genet, 1991, 225 (3):459-67), the Arabidopsis oleosin promoter (WO 98/45461), the Phaseolus vulgaris phaseolin promoter (U.S. Pat. No. 5,504,200), the brassica Bce4 promoter (WO 91/13980), the bean arc5 promoter, the carrot DcG3 promoter or the legumin B4 promoter (LeB4; Baeumlein et al., 1992, Plant Journal, 2 (2):233-9) and promoters which bring about seed-specific expression in monocotyledonous plants such as corn, barley, wheat, rye, rice etc. Advantageous seed-specific promoters are the sucrose binding protein promoter (WO 00/26388), the phaseolin promoter and the napin promoter. Suitable promoters worthy of note are the barley Ipt2 or Ipt1 gene promoter (WO 95/15389 and WO 95/23230) or those described in WO 99/16890 (promoters from the barley hordein gene, the rice glutelin gene, the rice oryzin gene, the rice prolamin gene, the wheat gliadin gene, wheat glutelin gene, the corn zein gene, the oats glutelin gene, the sorghum kasirin gene, the rye secalin gene).

In particular, multiparallel expression of the nucleic acids used in the process may be desired, alone or in combination with other genes or nucleic acids. Such expression cassettes can be introduced via simultaneous transformation of a plurality of individual expression constructs or, preferably, by combining a plurality of expression cassettes on one construct. It is also possible for a plurality of vectors to be transformed each with a plurality of expression cassettes and be transferred to the host cell.

Promoters which bring about plastid-specific expression are likewise particularly suitable.

Suitable promoters such as the viral RNA polymerase promoter are described in WO 95/16783 and WO 97/06250, and the Arabidopsis clpP promoter is described in WO 99/46394.

For strong expression of heterologous sequences in as many tissues as possible, especially including leaves, besides various of the abovementioned viral and bacterial promoters, preferably plant promoters of actin or ubiquitin genes such as, for example, the rice actin1 promoter are used. The sugar beet V-ATPase promoters (WO 01/14572) represent a further example of constitutive plant promoters. Examples which should be mentioned of synthetic constitutive promoters are the super promoter (WO 95/14098) and promoters derived from G boxes (WO 94/12015). A further possibility in some circumstances is also to utilize chemically inducible promoters, compare EP-A 388186, EP-A 335528, WO 97/06268. Also available for expression of genes in plants are leaf-specific promoters as described in DE-A 19644478, or photoregulated promoters such as, for example, the pea pete promoter.

Of the polyadenylation signals, particular mention should be made of the Poly-A addition sequence from the ocs gene or nos gene of Agrobacterium tumefaciens. Further regulatory sequences which are expedient where appropriate also include sequences which control the transport and/or the localization of the expression products (targeting). In this connection, mention should be made particularly of the signal peptide- or transit peptide-encoding sequences known per se. For example, it is possible with the aid of plastid transit peptide-encoding sequences to guide the expression product into the plastids of a plant cell. Plants particularly preferred as recipient plants are, as described above, those which can be transformed in an expedient manner. These include mono- and dicotyledonous plants. Particular mention should be made of agricultural crop plants such as cereals and grasses, e.g. Triticum spp., Zea mays, Hordeum vulgare, Hafer, Secale cereale, Oryza sativa, Pennisetum glaucum, Sorghum bicolor, Triticale, Agrosts spp., Cenchrus ciliaris, Dactylis glomerata, Festuca arundinacea, Lolium spp., Medicago spp. and Saccharum spp., legumes and oilseed crops, e.g. Brassica juncea, Brassica napus, Glycine max, Arachis hypogaea, Gossypium hirsutum, Cicer arietinum, Helianthus annuus, Lens culinaris, Linum usitatissimum, Sinapis alba, Trifolium repens and Vicia narbonensis, vegetables and fruits, e.g. bananas, grapes, Lycopersicon esculentum, asparagus, cabbage, water melons, kiwis, Solanum tuberosum, Beta vulgaris, cassaya and chicory, trees, e.g. Coffea species, Citrus spp., Eucalyptus spp., Piceaspp., Pinus spp. and Populus spp., medicinal plants and trees, and flowers. In a particular embodiment, the present invention relates to transgenic plants of the genus Arabidopsis, e.g. Arabidopsis thaliana and of the genus Oryza.

Vector DNA can be introduced into prokaryotic or eukaryotic cells by conventional transformation or transfection techniques. The terms “transformaton” and “transfection”, conjugation and transduction, as used herein, are intended to include a large number of processes known in the art for introducing foreign nucleic acid (e.g. DNA) into a host cell, including calcium phosphate or calcium chloride coprecipitation, DEAE-dextran-mediated transfection, PEG-mediated transfection, lipofection, natural competence, chemically mediated transfer, electroporation or particle bombardment. Processes suitable for the transformation or transfection of host cells, including plant cells, are to be found in Sambrook et al. (Molecular Cloning: A Laboratory Manual, 2nd edition, Cold Spring Harbor Laboratory, Cold Spring Harbor Laboratory Press, Cold Spring Harbor, N.Y., 1989) and other laboratory handbooks such as Methods in Molecular Biology, 1995, Vol. 44, Agrobacterium protocols, editors: Gartland and Davey, Humana Press, Totowa, N.J.

The term “nucleic acid (molecule or sequence)”, as used herein, may additionally include the untranslated sequence located at the 3′ end and at the 5′ end of the coding gene region: at least 500, preferably 200, particularly preferably 100, nucleotides of the sequence upstream of the 5′ end of the coding region and at least 100, preferably 50, particularly preferably 20, nucleotides of the sequence downstream of the 3′ end of the coding gene region. It is advantageous to take only the coding region for cloning and expression. An “isolated” nucleic acid molecule is separated from other nucleic acid molecules present in the natural source of the nucleic acid. An isolated” nucleic acid preferably has no sequences which naturally flank the nucleic acid in the genomic DNA of the organism from which the nucleic acid is derived (e.g. sequences located at the 5′ and 3′ ends of the nucleic acid). In various embodiments, the isolated nucleic acid molecule used in the process of the invention may comprise for example fewer than about 5 kb, 4 kb, 3 kb, 2 kb, 1 kb, 0.5 kb or 0.1 kb of nucleotide sequences which naturally flank the nucleic acid molecule in the genomic DNA of the cell from which the nucleic acid is derived.

The nucleic acid molecules used in the process, e.g. a nucleic acid molecule having a nucleotide sequence of SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25 or of a part thereof, can be isolated by use of standard techniques of molecular biology and the sequence information provided herein. It is also possible with the aid of comparison algorithms to identify for example a homologous sequence or homologous, conserved sequence regions at the DNA or amino acid level. These can be used as hybridization probe as well as standard hybridization techniques (as described, for example, in Sambrook et al., Molecular Cloning: A Laboratory Manual, 2nd edition, Cold Spring Harbor Laboratory, Cold Spring Harbor Laboratory Press, Cold Spring Harbor, N.Y., 1989) for isolating further nucleic acid sequences useful in the process. Moreover, a nucleic acid molecule comprising a complete sequence of SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25 or a part thereof can be isolated by polymerase chain reaction using oligonucleotide primers based on this sequence or parts thereof (e.g. a nucleic acid molecule comprising the complete sequence or a part thereof can be isolated by polymerase chain reaction using oligonucleotide primers constructed on the basis of this same sequence). For example, mRNA can be isolated from cells (e.g. by the guanidinium thiocyanate extraction process of Chirgwin et al. (1979) Biochemistry 18:5294-5299) and cDNA can be prepared using reverse transcriptase (e.g. Moloney MLV reverse transcriptase obtainable from Gibco/BRL, Bethesda, Md., or AMV reverse transcriptase obtainable from Seikagaku America, Inc., St. Petersburg, Fla.). Synthetic oligonucleotide primers for amplification using the polymerase chain reaction can be designed on the basis of one of the amino acid sequences depicted in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25 or with the aid of the amino acid sequences depicted in SEQ ID NO: 2, SEQ ID NO: 12, SEQ ID NO: 14, SEQ ID NO: 16, SEQ ID NO: 18, SEQ ID NO: 20. SEQ ID NO: 22. SEQ ID NO: 24, or SEQ ID NO: 26. A further Possibility is to identify, by protein sequence comparisons of threonine aldolases or lysine decarboxylases from various organisms, conserved regions from which in turn degenerate primers can then be derived. Such degenerate primers may be derived from the consensus sequences H[x]₂G[X]R[X]₁₉D[X]₇K[X]₂₇G, HXDGAR[X]₃A[X]₁₅D[X]₄CXSK[X]₄PXGS[X]₃G[X]₇A[X]₄K[X]₂GGGXRQXG, G[X]₄GIM[X]₄₅ M[X]₂RK[X]₂M[X]₁₁GGXG[X]₃E[X]₂E[X]₃W, or LG[X]₉LVYGG[X]₃GIMGXVA[X]₉G[X]₃GXIP[X]₂₄ MHXRK[X]₂ M[X]₆F[X]₃PGGXGTXEEp[X]₂E[X]₂TW[X]₂IG[X]₃ KP[X]₄N[X]₃FY[X]₁₄F. These degenerate primers can then be utilized for amplifying fragments of new threonine aldolases and/or lysine decarboxylases from other organisms by PCR. These fragments can then be utilized as hybridization probe for isolating the complete gene sequence. An alternative possibility is to isolate the missing 5′ and 3′ sequences by means of RACE-PCR. A nucleic acid of the invention can be amplified using cDNA or, alternatively, genomic DNA as template and suitable oligonucleotide primers in standard PCR amplification techniques. The nucleic acid amplified in this way can be cloned into a suitable vector and characterized by DNA sequence analysis. Oligonucleotides corresponding to a nucleotide sequence used in the process can be prepared by standard synthetic processes, for example using an automatic DNA synthesizer.

Nucleic acid molecules advantageous for the process of the invention can be isolated on the basis of their homology with the nucleic acids disclosed herein, using the sequences or a part thereof as hybridization probe in standard hybridization techniques under stringent hybridization conditions. In these cases it is possible for example to use isolated nucleic acid molecules which are at least 15 nucleotides long and hybridize under stringent conditions with the nucleic acid molecules comprising a nucleotide sequence of SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25. Nucleic acids of at least 25, 50, 100, 250 or more nucleotides can also be used. The term “hybridizes under stringent conditions”, as used herein, is intended to describe hybridization and washing conditions under which nucleotide sequences which are at least 60% homologous with one another usually remain hybridized together. The conditions are preferably such that sequences which are at least about 65%, more preferably at least about 70% and even more preferably at least about 75% or more homologous with one another usually remain hybridized together. Homolog or homology mean for the purposes of the invention identical or identity. These stringent conditions are known to the skilled worker and can be found in Current Protocols in Molecular Biology, John Wiley & Sons, N.Y. (1989), 6.3.1-6.3.6. A preferred, non-restrictive example of stringent hybridization conditions are hybridizations in 6× sodium chloride/sodium citrate (=SSC) at about 45° C., followed by one or more washing steps in 0.2×SSC, 0.1% SDS at 50 to 65° C. The skilled worker is aware that these hybridization conditions differ according to the type of nucleic acid and, if for example organic solvents are present, with regard to the temperature and concentration of the buffer. The temperature differs for example under “standard hybridization conditions” depending on the type of nucleic acid between 42° C. and 58° C. in aqueous buffer with a concentration of from 0.1 to 5×SSC (pH 7.2). If organic solvent is present in the abovementioned buffer, for example 50% formamide, the temperature under standard conditions is about 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably for example 0.1×SSC and 30° C. to 55° C., preferably between 45° C. and 55° C. The aforementioned hybridization temperatures are intended for example for a nucleic acid with a length of about 100 bp (=base pairs) and a G+C content of 50% in the absence of formamide. The skilled worker is aware of how the necessary hybridization conditions can be determined from textbooks such as the aforementioned or from the following textbooks Sambrook et al., “Molecular Cloning”, Cold Spring Harbor Laboratory, 1989; Hames and Higgins (editors) 1985, “Nucleic Acids Hybridization: A Practical Approach”, IRL Press at Oxford University Press, Oxford; Brown (editors) 1991, “Essential Molecular Biology: A Practical Approach”, IRL Press at Oxford University Press, Oxford.

To determine the percentage homology (=identity) of two amino acid sequences (e.g. of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10) or of two nucleic acids (e.g. of sequence SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25), the sequences are aligned for optimal comparison purposes (e.g. gaps can be introduced in the sequence of one protein or nucleic acid to produce optimal alignment with the other protein or other nucleic acid). The amino acid residues or nucleotides at the corresponding amino acid positions or nucleotide positions are then compared. When a position in one sequence is occupied by the same amino acid residue or the same nucleotide as the corresponding position in the other sequence, then the molecules are homologous at this position (i.e. as used herein amino acid or nucleic acid “homology” is equivalent to amino acid or nucleic acid “identity”). The percentage homology between the two sequences is a function of the number of identical positions shared by the sequences (i.e. % homology=number of identical positions/total number of positions×100). The terms homology and identity are thus to be regarded as synonymous.

An isolated nucleic acid molecule coding for a threonine aldolase or lysine decarboxylase homologous to a protein sequence of SEQ ID NO: 2, SEQ ID NO: 12, SEQ ID NO: 14, SEQ ID NO: 16, SEQ ID NO: 18, SEQ ID NO: 20, SEQ ID NO: 22, SEQ ID NO: 24 or SEQ ID NO: 26 or the sequences SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 can be generated by introducing one or more nucleotide substitutions, additions or deletions into a nucleotide sequence of SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 or into the nucleic acid sequences derived from the aforementioned amino acid sequences so that one or more amino acid substitutions, additions or deletions are introduced into the encoded protein. Mutations can be introduced into one of the sequences of SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 by standard techniques, such as site-specific mutagenesis and PCR-mediated mutagenesis. Preferably, conservative amino acid substitutions are produced at one or more of the predicted nonessential amino acid residues. A “conservative amino acid substitution” is one in which the amino acid residue is replaced by an amino acid residue having a similar side chain. Families of amino acid residues having similar side chains have been defined in the art. These families include amino acids having basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine). A predicted nonessential amino acid residue in a protein sequence such as SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24 or SEQ ID NO: 26 is thus preferably replaced by another amino acid residue from the same side-chain family. Alternatively, in another embodiment, the mutations can be introduced randomly along all or part of the coding sequence, e.g. by saturation mutagenesis, and the resulting mutants can be screened for their biological activity, i.e. amino acid production, in order to identify mutants which retain the biological activity or have increased it. After mutagenesis of one of the sequences of SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 or of the nucleic acid sequence which can be derived from the aforementioned sequences, the encoded protein can be expressed recombinantly, and the activity of the protein can be determined for example using the assays described herein.

Homologs of the nucleic acid sequences used with the sequence SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 or the nucleic acid sequences derived from the sequences SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 mean, for example, allelic variants having at least about 30 to 50%, preferably at least about 50 to 70%, more preferably at least about 70 to 80%, 80 to 90% or 90 to 95% and even more preferably at least about 95%, 96%, 97%, 98%, 99% or more homology with one of the nucleotide sequences shown in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 or the aforementioned derived nucleic acid sequences or their homologs, derivatives or analogs or parts thereof. In addition, isolated nucleic acid molecules of a nucleotide sequence which hybridize onto one of the nucleotide sequences shown in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25, the derived nucleic acid sequences or a part thereof are, e.g. hybridizes under stringent conditions. Allelic variants include in particular functional variants which can be obtained by deletion, insertion or substitution of nucleotides from/in the sequence depicted in SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 or the derived nucleic acid sequences, the intention being, however, that the enzymic activity or the biological activity of the synthesized proteins originating therefrom advantageously be retained for the insertion of one or more genes. Proteins which still have the essential enzymatic activity of threonine aldolase, i.e. their activity is negligibly reduced, means proteins having at least 10%, preferably 20%, particularly preferably 30%, very particularly preferably 40%, of the original biological or enzymic activity, advantageously compared with the protein encoded by SEQ ID NO: 2, SEQ ID NO: 12, SEQ ID NO: 14, SEQ ID NO: 16, SEQ ID NO: 18, SEQ ID NO: 20, SEQ ID NO: 22, SEQ ID NO: 24 or SEQ ID NO: 26.

Homologs of SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 or of the derived sequences also mean, for example, bacterial, fungal and plant homologs, truncated sequences, single-stranded DNA or RNA of the coding and noncoding DNA sequence.

Homologs of SEQ ID NO: 1, SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 or of the derived sequences also mean derivatives such as, for example, promoter variants. The promoters upstream of the indicated nucleotide sequences may be modified by one or more nucleotide exchanges, by insertion(s) and/or deletion(s) without, however, impairing the functionality or activity of the promoters. It is additionally possible for the activity of the promoters to be increased by modifying their sequence, or for them to be completely replaced by more active promoters, even from heterologous organisms.

The aforementioned nucleic acids and protein molecules having threonine aldolase activity and/or lysine decarboxylase activity which are involved in the amino acid metabolism are used to increase the yield, production and/or efficiency of production of a desired compound or a decrease in unwanted compounds.

The organisms used in the process of the invention are grown or cultured in a manner known to the skilled worker depending on the host organism. Microorganisms are ordinarily grown in a liquid medium which contains a carbon source, usually in the form of sugars, a nitrogen source, usually in the form of organic nitrogen sources such as yeast extract or salts such as ammonium sulfate, trace elements such as iron, manganese, magnesium salts and, where appropriate, vitamins, at temperatures between 0° C. and 100° C., preferably between 10° C. to 60° C., while passing in oxygen. The pH of the nutrient liquid can be kept at a fixed value during this, i.e. controlled during the cultivation, or not. The cultivation can be carried out batchwise, semibatchwise or continuously. Nutrients can be introduced at the start of the fermentation or be subsequently fed in semicontinuously or continuously. The produced amino acids can be isolated from the organisms by processes known to the skilled worker. For example by extraction, salt precipitation and/or ion exchange chromatography. The organisms may also for this purpose be disrupted beforehand.

The process of the invention is, when the host organisms are microorganisms, advantageously carried out at a temperature between 0° C. to 95° C., preferably between 10° C. to 85° C., particularly preferably between 15° C. to 75° C., very particularly preferably between 15° C. to 45° C.

The pH is advantageously kept at between pH 4 and 12, preferably between pH 6 and 9, particularly preferably between pH 7 and 8, during this.

The process of the invention can be operated batchwise, semibatchwise or continuously. A summary of known cultivation methods is to be found in the textbook by Chmiel (Bioprozeβtechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used must meet the requirements of the respective strains in a suitable manner. Descriptions of culture media for various microorganisms are present in the handbook “Manual of Methods for General Bacteriology” of the American Society for Bacteriology (Washington D.C., USA, 1981).

These media which can be employed according to the invention include, as described above, usually one or more carbon sources, nitrogen sources, inorganic salts, vitamins and/or trace elements.

Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars can also be added to the media via complex compounds such as molasses, or other byproducts of sugar refining. It may also be advantageous to add mixtures of various carbon sources. Other possible carbon sources are oils and fats such as, for example, soybean oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol and/or organic acids such as, for example, acetic acid and/or lactic acid.

Nitrogen sources are usually organic or inorganic nitrogen compounds or materials which contain these compounds. Examples of nitrogen sources include ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as corn steep liquor, soybean meal, soybean protein, yeast extract, meat extract and others. The nitrogen sources may be used singly or as a mixture.

Inorganic salt compounds which may be present in the media include the chloride, phosphorus or sulfate salts of calcium, magnesium, sodium, cobalt, molybdenum, potassium, manganese, zinc, copper and iron.

For preparing sulfur-containing fine chemicals, in particular methionine, it is possible to use as sulfur source inorganic sulfur-containing compounds such as, for example, sulfates, sulfites, dithionites, tetrathionates, thiosulfates, sulfides or else organic sulfur compounds such as mercaptans and thiols.

It is possible to use as phosphorus source phosphoric acid, potassium dihydrogenphosphate or dipotassium hydrogenphosphate or the corresponding sodium-containing salts.

Chelating agents can be added to the medium in order to keep the metal ions in solution. Particularly suitable chelating agents include dihydroxyphenols such as catechol or protocatechuate, or organic acids such as citric acid.

The fermentation media employed according to the invention for cultivating microorganisms normally also contain other growth factors such as vitamins or growth promoters, which include, for example, biotin, riboflavin, thiamine, folic acid, nicotinic acid, pantothenate and pyridoxine.

Growth factors and salts are often derived from complex media components such as yeast extract, molasses, corn steep liquor and the like. Suitable precursors can moreover be added to the culture medium. The exact composition of the media compounds depends greatly on the particular experiment and is chosen individually for each specific case. Information about media optimization is obtainable from the textbook “Applied Microbiol. Physiology, A Practical Approach” (editors P. M. Rhodes, P. F. Stanbury, IRL Press (1997) pp. 53-73, ISBN 0 19 963577 3). Growth media can also be purchased from commercial suppliers such as Standard 1 (Merck) or BHI (Brain heart infusion, DIFCO) and the like.

All media components are sterilized either by heat (1.5 bar and 121° C. for 20 min) or by sterilizing filtration. The components can be sterilized either together or, if necessary, separately. All media components can be present at the start of the cultivation or optionally be added continuously or batchwise.

The temperature of the culture is normally between 15° C. and 45° C., preferably at 25° C. to 40° C., and can be kept constant or changed during the experiment. The pH of the medium should be in the range from 5 to 8.5, preferably around 7. The pH for the cultivation can be controlled during the cultivation by adding basic compounds such as sodium hydroxide, potassium hydroxide, ammonia or aqueous ammonia or acidic compounds such as phosphoric acid or sulfuric acid. Foaming can be controlled by employing antifoams such as, for example, fatty acid polyglycol esters. The stability of plasmids can be maintained by adding to the medium suitable substances having a selective effect, for example antibiotics. Aerobic conditions are maintained by introducing oxygen or oxygen-containing gas mixtures such as, for example, ambient air into the culture. The temperature of the culture is normally from 20° C. to 45° C. and preferably from 25° C. to 40° C. The culture is continued until formation of the desired product is at a maximum. This aim is normally achieved within 10 hours to 160 hours.

The fermentation broths obtained in this way, containing in particular L-methionine and/or L-lysine, advantageously L-methionine, normally have a dry matter content of from 7.5 to 25% by weight.

Sugar-limited fermentation is additionally advantageous, at least at the end, but especially over at least 30% of the fermentation time. This means that the concentration of utilizable sugar in the fermentation medium is kept at, or reduced to, ≧0 to 3 g/l during this time.

The fermentation broth is then processed further. Depending on requirements, the biomass can be removed entirely or partly by separation methods, such as, for example, centrifugation, filtration, decantation or a combination of these methods, from the fermentation broth or left completely in it.

The fermentation broth can then be thickened or concentrated by known methods, such as, for example, with the aid of a rotary evaporator, thin-film evaporator, falling film evaporator, by reverse osmosis or by nanofiltration. This concentrated fermentation broth can then be worked up by freeze drying, spray drying, spray granulation or by other processes.

However, it is also possible to purify the amino acid further. For this purpose, the product-containing broth after removal of the biomass is subjected to a chromatography on a suitable resin, in which case the desired product or the impurities are retained wholly or partly on the chromatography resin. These chromatography steps can be repeated if necessary, using the same or different chromatography resins. The skilled worker is familiar with the choice of suitable chromatography resins and their most effective use. The purified product can be concentrated by filtration or ultrafiltrabon and stored at a temperature at which the stability of the product is a maximum.

The identity and purity of the isolated compound(s) can be determined by prior art techniques. These include high performance liquid chromatography (HPLC), spectroscopic methods, mass spectrometry, staining methods, thin-layer chromatography, NIRS, enzyme assay or microbiological assays. These analytical methods are summarized in: Patek et al. (1994) Appl. Environ. Microbiol. 60:133-140; Malakhova et al. (1996) Biotekhnologiya 11 27-32; and Schmidt et al. (1998) Bioprocess Engineer. 19:67-70. Ulmann's Encyclopedia of Industrial Chemistry (1996) Vol. A27, VCH: Weinheim, pp. 89-90, pp. 521-540, pp. 540-547, pp. 559-566, 575-581 and pp. 581-587; Michal, G (1999) Biochemical Pathways: An Atlas of Biochemistry and Molecular Biology, John Wiley and Sons; Fallon, A et al. (1987) Applications of HPLC in Biochemistry in: Laboratory Techniques in Biochemistry and Molecular Biology, Vol. 17.

The amino acids obtained in the process are suitable as starting material for synthesizing further products of value. They can be used for example in combination with one another or alone for producing drugs, human foods, animal feeds or cosmetics.

The transfer of foreign genes into the genome of a plant is referred to, as described above, as transformation. In this case, the methods described for transformation and regeneration of plants from plant tissues or plant cells are utilized for transient or stable transformation. Suitable methods are protoplast transformation by polyethylene glycol-induced DNA uptake, the biolistic method with the gene gun—the so-called particle bombardment method, electroporation, incubation of dry embryos in DNA-containing solution, microinjection and Agrobacterium-mediated gene transfer. Said processes are described, for example, in B. Jenes et al., Techniques for Gene Transfer, in: Transgenic Plants, Vol. 1, Engineering and Utilization, edited by S. D. Kung and R. Wu, Academic Press (1993) 128-143 and in Potrykus Annu. Rev. Plant Physiol. Plant Molec. Biol. 42 (1991) 205-225. The construct to be expressed is preferably cloned into a vector which is suitable for transforming Agrobacterium tumefaciens, for example pBin19 (Bevan et al., Nucl. Acids Res. 12 (1984) 8711). Agrobacteria transformed with such a vector can then be used in a known manner for transforming plants, especially crop plants, such as, for example, tobacco plants, by, for example, bathing wounded leaves or pieces of leaves in a solution of agrobacteria and then cultivating in suitable media. Transformation of plants with Agrobacterium tumefaciens is described for example by Höfgen and Willmitzer in Nucl. Acid Res. (1988) 16, 9877 or is disclosed inter alia in F. F. White, Vectors for Gene Transfer in Higher Plants; in Transgenic Plants, Vol. 1, Engineering and Utilization, edited by S. D. Kung and R. Wu, Academic Press, 1993, pp. 15-38.

Marker genes are advantageously used for selection for successful introduction of the nucleic acids of the invention into a host organism. These marker genes make it possible to identify successful introduction of the nucleic acids of the invention by a number of different principles, for example by visual recognition with the aid of fluorescence, luminescence or in the wavelength range of light which is visible to humans, via a herbicide or antibiotic resistance, via so-called nutritional (auxotrophic markers) or antinutritional markers, by enzyme assays or via phyto hormones. Examples of such markers which may be mentioned here are the GFP (=green fluorescent protein); the luciferin/luceferace system; β-galactosidase with its colored substrates e.g. X-Gal; herbicide resistances to, for example, imidazolinone, glyphosate, phosphothricin or sulfonylurea; antibiotic resistances to, for example, bleomycin, hygromycin, streptomycin, kanamycin, tetracycline, chloramphenicol, ampicillin, gentamicin, geneticin (G418), spectinomycin or blasticidin to mention only a few; nutritional markers such as utilization of mannose or xylose or antinutritional markers such as 2-deoxyglucose resistance. This list represents a small section of possible markers. Markers of these types are well known to the skilled worker. Different markers are preferred, depending on organism and selection method.

It is known about stable or transient integration of nucleic acids in plant cells that, depending on the expression vector used and transfection technique used, only a small part of the cells takes up the foreign DNA and, if desired, integrates it in their genome. For identification and selection of these integrants, usually a gene which encodes a selectable marker (e.g. antibiotic resistance) is introduced together with the gene of interest into the host cells. Preferred selectable markers include in plants those which confer resistance to a herbicide such as glyphosphate or glufosinate. Further suitable markers are, for example, markers which encode genes which are involved in biosynthetic pathways of, for example, sugars or amino acids, such as β-galactosidase, ura3 or ilv2. Markers encoding genes such as luciferase, gfp or other fluorescence genes are likewise suitable. These markers can be used in mutants in which these genes are not functional because, for example, they have been deleted by conventional methods. Markers which encode a nucleic acid encoding a selectable marker can moreover be introduced into a host cell on the same vector as that coding for the threonine aldolases and/or lysine decarboxylases used in the process, or can be introduced on a separate vector. Cells stably transfected with the introduced nucleic acid can be identified for example by selection (e.g. cells which have integrated the selectable marker survive, whereas the other cells die).

Since, usually, the marker genes, specifically the antibiotic and herbicide resistance gene, are no longer required or are unwanted in the transgenic host cell after successful introduction of the nucleic acids, techniques making it possible to delete or excise these marker genes are advantageously used in the process of the invention for introducing the nucleic acids. One such method is so-called cotransformation. In cotransformation, two vectors are used simultaneously for the transformation, one vector harboring the nucleic acids of the invention and the second one harboring the marker gene(s). A large part of the transformants acquires or contains both vectors in the case of plants (up to 40% of the transformants and more). It is then possible to remove the marker genes from the transformed plant by crossing. A further method uses marker genes integrated into a transposon for the transformation together with the desired nucleic acids (so-called Ac/Ds technology). In some cases (about 10%), after successful transformation the transposon jumps out of the genome of the host cell and is lost. In a further number of cases, the transposon jumps into another site. In these cases, outcrossing of the marker gene again is necessary. Microbiological techniques enabling or facilitating detection of such events have been developed. A further advantageous method uses so-called recombination systems which have the advantage that it is possible to dispense with outcrossing. The best-known system of this type is the so-called Cre/lox system. Cre1 is a recombinase which deletes the sequences located between the loxP sequence. If the marker gene is integrated between the loxP sequence, it is deleted by expression of the recombinase after successful transformation. Further recombinase systems are the HIN/HIX, the FLP/FRT and the REP/STB systems (Tribble et al., J. Biol. Chem., 275, 2000: 22255-22267; Velmurugan et al.,. J. Cell Biol., 149, 2000: 553-566). Targeted integration of the nucleic acid sequences of the invention into the plant genome is also possible in principle but less preferred because of the large amount of work involved. These methods are, of course, also applicable to microorganisms such as yeasts, fungi or bacteria.

Agrobacteria transformed with an expression vector of the invention can likewise be used in a known manner for transforming plants such as test plants such as Arabidopsis or crop plants such as, for example, cereals, corn, oats, rye, barley, wheat, soybean, rice, cotton, sugar beet, canola, sunflower, flax, hemp, potato, tobacco, tomato, carrot, paprika, oilseed rape, tapioca, cassaya, arrowroot, tagetes, alfalfa, lettuce and the various tree, nut and grape species, especially oil-containing crop plants such as soybean, peanut, castor oil plant, sunflower, corn, cotton, flax, oilseed rape, coconut, oil palm, safflower (Carthamus tinctorius) or cocoa bean, e.g. by bathing wounded leaves or pieces of leaves in a solution of agrobacteria and then cultivating in suitable media.

The genetically modified plant cells can be regenerated by all methods known to the skilled worker. Appropriate methods can be found in the abovementioned publications by S. D. Kung and R. Wu, Potrykus or Höfgen and Willmitzer.

Besides the transformation of somatic cells, which must then be regenerated to plants, it is also possible to transform cells of plant meristems and, in particular, those cells which develop into gametes. In this case, the transformed gametes lead to transgenic plants by the route of natural plant development. Thus, for example, seeds of Arabidopsis are treated with agrobacteria, and seeds are obtained from the plants developing therefrom, which seeds show a certain transformation rate and are therefore transgenic (Feldman, K A and Marks M D (1987), Agrobacterium-mediated transformation of germinating seeds of Arabidopsis thaliana: a non tissue culture approach. Mol Gen Genet 208:274-289; Feldmann K (1992) T-DNA insertion mutagenesis in Arabidopsis: seed infection transformation. In C Koncz, N-H Chua and J Shell, eds, Methods in Arabidopsis Research. Word Scientific, Singapore, pp. 274-289). Alternative methods are based on repeated removal of the inflorescences and incubation of the severed site in the center of the rosette with transformed agrobacteria, likewise making it possible to obtain transformed seeds later (Chang, S S, Park S K, Kim, B C, Kang, B J, Kim D U and Nam, H G (1994) Stable genetic transformation of Arabidopsis thaliana by Agrobacterium inoculation in planta. Plant J. 5: 551-558; Katavic, V, Haughn, G W, Reed, D, Martin, M and Kunst, L (1994) In planta transformation of Arabidopsis thaliana. Mol Gen Genet, 245: 363-370). However, the method of vacuum infiltration with its modifications such as floral dip is particularly efficient. In the vacuum infiltration of Arabidopsis, whole plants are treated with a suspension of agrobacterium in vacuo (Bechthold, N, Ellis, J, and Pelletier, G (1993) In planta Agrobacterium-mediated gene transfer by infiltration of adult Arabidopsis thaliana plants. C R Acad Sci Paris Life Sci, 316: 1194-1199), while in the floral dip method the developing flower tissue is briefly incubated in a suspension of agrobacteria mixed with a surfactant (Clough, S J and Bent, A F (1998) Floral dip: a simple method for Agrobacterium-mediated transformation of Arabidopsis thaliana. The Plant J. 16, 735-743). In both cases, a certain percentage of transgenic seeds are harvested and can be distinguished from non-transgenic seeds by cultivation under the selective conditions described above.

A further aspect of the invention therefore relates to transgenic organisms transformed with at least one nucleic acid sequence or expression cassette of the invention or with a vector of the invention, and to cells, cell cultures, tissues, parts—such as, for example in the case of plant organisms, leaves, roots, etc.—or propagation material derived from such organisms. The terms ghost organism”, “host cell”, “recombinant (host) organism”, “recombinant (host) cell”, “transgenic (host) organism” and “transgenic (host) cell” are used interchangeably herein. It is self-evident that these terms relate not only to the particular host organism or to the particular target cell but also to the progeny or potential progeny of these organisms or cells. Since certain modifications may occur in subsequent generations owing to mutation or environmental effects, these progeny are not necessarily identical to the parental cell but are still included within the scope of the term as used herein.

The amino acid sequences classified in SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 are a further aspect of the invention.

This invention is illustrated further by the following examples, which are not to be regarded as restrictive. The contents of all the references, patent applications, patents and published patent applications cited in this patent application are incorporated herein by reference.

### EXAMPLES

### Example 1

### Cloning of SEQ ID NO: 1 into Escherichia coli

SEQ ID NO: 1 was cloned by well-known and well-established methods (see, for example, Sambrook, J. et al. (1989) “Molecular Cloning: A Laboratory Manual”. Cold Spring Harbor Laboratory Press or Ausubel, F. M. et al. (1994) “Current Protocols in Molecular Biology”, John Wiley & Sons) into the plasmids pBR322 (Sutcliffe, J. G. (1979) Proc. Natl. Acad. Sci. USA, 75: 3737-3741); pACYC177 (Change & Cohen (1978) J. Bacteriol. 134:1141-1156); plasmids of the pBS series (pBSSK+, pBSSK− and others; Stratagene, LaJolla, USA) or cosmids such as SuperCos1 (Stratagene, LaJolla, USA) or Lorist6 (Gibson, T. J. Rosenthal, A, and Waterson, R. H. (1987) Gene 53: 283-286) for expression in E. coli.

The sequences SEQ ID NO: 11, SEQ ID NO: 13, SEQ ID NO: 15, SEQ ID NO: 17, SEQ ID NO: 19, SEQ ID NO: 21, SEQ ID NO: 23 or SEQ ID NO: 25 were cloned analogously.

### Example 2

### DNA Sequencing and Computer Function Analysis

The DNA sequencing was carried out by standard methods, in particular the chain termination method with ABI377 sequencers (see, for example, Fleischman, R. D. et al. (1995) “Whole-genome Random Sequencing and Assembly of Haemophilus Influenzae Rd.”, Science 269; 496-512).

### Example 3

### In Vivo Mutagenesis

Mutagenesis of Corynebacterium glutamicum in vivo can be carried out by passing a plasmid (or other vector) DNA through E. coli or other microorganisms (e.g. Bacillus spp. or yeasts such as Saccharomyces cerevisiae) unable to maintain the integrity of their genetic information. Usual mutator strains have mutations in the genes for the DNA repair system [e.g. mutHLS, mutD, mutT, etc., for comparison, see Rupp, W. D. (1996) DNA repair mechanisms in Escherichia coli and Salmonella, pp. 2277-2294, ASM: Washington]. These strains are known to the skilled worker. The use of these strains is explained for example in Greener, k and Callahan, M. (1994) Strategies 7; 32-34.

### Example 4

### DNA Transfer Between Escherichia coli and Corynebacterium glutamicum

Several Corynebacterium and Brevibacterium species contain endogenous plasmids (such as, for example, pHM1519 or pBL1) which undergo autonomous replication (for a review, see, for example, Martin, J. F. et al. (1987) Biotechnology 5: 137-146). Shuttle vectors for Escherichia coli and Corynebacterium glutamicum can easily be constructed by means of standard vectors for E. coli (Sambrook, J. et al., (1989), “Molecular Cloning: A Laboratory Manual”, Cold Spring Harbor Laboratory Press or Ausubel, F. M. et al. (1994) “Current Protocols in Molecular Biology”, John Wiley & Sons), to which an origin of replication for and a suitable marker from Corynebacterium glutamicum is added. Such origins of replication are preferably taken from endogenous plasmids isolated from Corynebacterium and Brevibacterium species. Particular use as transformation markers for these species are genes for kanamycin resistance (such as those derived from the Tn5 or Tn-903 transposon) or for chloramphenicol (Winnacker, E. L. (1987) “From Genes to Clones—Introduction to Gene Technology, VCH, Weinheim). There are numerous examples in the literature of the preparation of a large number of shuttle vectors which are replicated in E. coli and C. glutamicum, and which can be used for various purposes, including gene overexpression (see, for example, Yoshihama, M. et al. (1) J. Bacteriol. 162: 591-597, Martin, J. F. et al., (1987) Biotechnology, 5: 137-146 and Eikmanns, B. J. et al. (1992) Gene 102: 93-98). Suitable vectors which replicate in coryneform bacteria are, for example, pZl (Menkel et al., Appl. Environ. Microbiol., 64, 1989: 549-554), pEkEx1 (Eikmanns et al., Gene 102, 1991: 93-98) or pHS2-1 (Sonnen et al, Gene 107, 1991: 69-74). These vectors are based on cryptic plasmids pHM1519, pBL1 or pGA1. Other plasmid vectors such as, for example, those based on pCG4 (U.S. Pat. No. 4,489,160), pNG2 (Serwold-Davis et al., FEMS Microbiol. Lett., 66, 1990:119-124) or pAG1 (U.S. Pat. No. 5,158,891) can be used in a similar way.

It is possible by standard methods to clone a gene of interest into one of the shuttle vectors described above, and to introduce such hybrid vectors into Corynebacterium glutamicum strains. Transformation of C. glutamicum can be achieved by protoplast transformation (Kastsumata, R. et al., (1984) J. Bacteriol. 159, 306-311), electroporation (Liebl, E. et al., (1989) FEMS Microbiol. Letters, 53: 399-303) and, in cases where specific vectors are used, also by conjugation (as described, for example, in Schafer, A., et (1990) J. Bacteriol. 172: 1663-1666). It is likewise possible to transfer the shuttle vectors for C. glutamicum to E. coli by preparing plasmid DNA from C. glutamicum (by standard methods known in the art) and transforming it into E. coli. This transformation step can take place using standard methods, but an Mcr-deficient E. coli strain is advantageously used, such as NM522 (Gough & Murray (1983) J. Mol. Biol. 166: 1-19).

If it is intended, advantageously, that the transformed sequence(s) be integrated into the genome of the coryneform bacteria, standard techniques for this are also known to the skilled worker. For example, plasmid vectors like those described by Remscheid et al. (Appl. Environ. Microbiol., 60, 1994: 126-132) for the duplication or amplification of the hom-thrB operon are used for this purpose. In this method, the complete gene is cloned into a plasmid vector able to replicate in a host such as E. coli but not in C. glutamicum. Examples of suitable vectors are pSUP301 (Simon et al., Bio/Technology 1, 1983: 784-791), pKIBmob or pK19mob (Schafer et al., Gene 145, 1994: 69-73), pGEM-T (Promega Corp., Madison, Wis., USA), pCR2.1-TOPO (Schuman, J. Biol. Chem., 269, 1994: 32678-32684, U.S. Pat. No. 5,487,993), pCR® Blunt (from Invitrogen, Groningen, The Netherlands) or pEM1 (Schrumpf et al., J. Bacteriol., 173, 1991: 4510-4516).

### Example 5

### Determination of the Expression of the Mutant/Transgenic Protein

Observations of the activity of a mutated or transgenic protein in a transformed host cell are based on the fact that the protein is expressed in a similar way and in similar quantity to the wild-type protein. A suitable method for determining the transcription rate of the mutant or transgenic gene (an indicator of the quantity of mRNA available for translation of the gene product) is to carry out a Northern blot (see, for example, Ausubel et al., (1988) Current Protocols in Molecular Biology, Wiley: New York), where a primer which is designed so that it binds to the gene of interest is provided with a detectable (usually radioactive or chemiluminescent) label so that—when the complete RNA is extracted from a culture of the organism, fractionated on a gel, transferred to a stable matrix and incubated with this probe—the binding and the quantity of the binding of the probe indicates the presence and also the quantity of mRNA for this gene. This information is a demonstration of the extent of transcription of the gene. Complete cellular RNA can be isolated from Corynebacterium glutamicum by various methods known in the art, as described in Bormann, E. R. et al., (1992) Mol. Microbiol. 6: 317-326.

The presence or the relative quantity of protein translated from this mRNA can be determined by employing standard techniques such as Western blotting (see, for example, Ausubel et al. (1988) “Current Protocols in Molecular Biology”, Wiley, New York). In this method, all cellular proteins are extracted, separated by gel electrophoresis, transferred to a matrix such as nitrocellulose, and incubated with a probe, such as an antibody, which binds specifically to the desired protein. This probe is usually provided directly or indirectly with a chemiluminescent or colorimetric label which can easily be detected. The presence and the observed quantity of labels indicates the presence and the quantity of the mutant protein which is sought in the cell.

### Example 6

### Growth of Genetically Modified Corynebacterium glutamicum—Media and Cultivation Conditions

Genetically modified corynebacteria are cultured in synthetic or natural growth media. A number of different growth media for corynebacteria are known and easily obtainable (Lieb et al. (1989) Appl. Microbiol. Biotechnol. 32: 205-210; von der Osten et al. (1998) Biotechnology Letters 11: 11-16; Patent DE 4 120 867; Liebl (1992) “The Genus Corynebacterium”, in: The Procaryotes, Vol. II, Balows, A., et al., editors, Springer-Verlag). These media consist of one or more carbon sources, nitrogen sources, inorganic salts, vitamins and trace elements. Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars can also be added to the media via complex compounds such as molasses, or other byproducts of sugar refining. It may also be advantageous to add mixtures of various carbon sources. Other possible carbon sources are alcohols and/or organic acids such as methanol, ethanol, acetic acid or lactic acid. Nitrogen sources are usually organic or inorganic nitrogen compounds or materials which contain these compounds. Examples of nitrogen sources include ammonia gas, aqueous ammonia solutions or ammonium salts such as NH₄Cl or (NH₄)₂SO₄, NH₄OH, nitrates, urea, amino acids or complex nitrogen sources such as corn steep liquor, soybean meal, soybean protein, yeast extracts, meat extracts and others. Mixtures of the aforementioned nitrogen sources may also advantageously be used.

Inorganic salt compounds which may be present in the media include the chloride, phosphorus or sulfate salts of calcium, magnesium, sodium, cobalt, molybdenum, potassium, manganese, zinc, copper and iron. Chelating agents can be added to the medium in order to keep the metal ions in solution. Particularly suitable chelating agents include dihydroxyphenols such as catechol or protocatechuate, or organic acids such as citric acid. The media normally also contain other growth factors such as vitamins or growth promoters, which include, for example, biotin, riboflavin, thiamine, folic acid, nicotinic acid, pantothenate and pyridoxine. Growth factors and salts are often derived from complex media components such as yeast extract, molasses, corn steep liquor and the like. The exact composition of the media compounds depends greatly on the particular experiment and is chosen individually for each specific case. Information about media optimization is obtainable, for example, from the textbook “Applied Microbiol. Physiology, A Practical Approach” (editors P. M. Rhodes, P. F. Stanbury, IRL Press (1997) pp. 53-73, ISBN 0 19 963577 3). Growth media can also be purchased from commercial suppliers such as Standard 1 (Merck) or BHI (Brain heart infusion, DIFCO) and the like.

All media components are sterilized either by heat (1.5 bar and 121° C. for 20 min) or by sterilizing filtration. The components can be sterilized either together or, if necessary, separately. All media components can be present at the start of the cultivation or optionally be added continuously or batchwise.

The cultivation conditions are defined separately for each experiment. The temperature should be between 15° C. and 45° C. and can be kept constant or changed during the experiment. The pH of the medium should be in the range from 5 to 8.5, preferably around 7.0, and can be maintained by adding buffers to the media. One example of a buffer for this purpose is a potassium phosphate buffer. Synthetic buffers such as MOPS, HEPES; ACES etc. can be used alternatively or simultaneously. The cultivation pH can be kept constant during the cultivation also by adding, for example, NaOH or NH₄OH. If complex media components such as yeast extract are used, the requirement for additional buffers is reduced because many complex compounds have a high buffer capacity. If a fermenter is used for cultivating microorganisms, the pH can also be controlled with gaseous ammonia.

The incubation time is usually in a range from several hours up to several days. This time is selected so that the maximum quantity of product accumulates in the fermentation broth. The disclosed growth experiments can be carried out in a large number of containers such as microtiter plates, glass tubes, glass flasks or glass or metal fermenters of various sizes. For screening a large number of clones, the microorganisms should be cultured in microtiter plates, glass tubes or shaker flasks either with or without baffles. 100 ml shaker flasks are preferably used and are charged with 10% (based on volume) of the required growth medium. The flasks should be shaken on an orbital shaker (amplitude 25 mm) with a speed in the range from 100-300 rpm. Evaporation losses can be reduced by maintaining a moist atmosphere; alternatively, a mathematical correction should be carried out for the evaporation losses.

If genetically modified clones are investigated, there should also be testing of an unmodified control clone or a control clone which contains the basic plasmid without insert. If a transgenic sequence is to be expressed, in this case too a control clone should also advantageously be tested. The medium is advantageously inoculated to an OD600 of 0.5-1.5, using cells cultured on agar plates, such as CM plates (10 g/l glucose, 2.5 g/l NaCl, 2 g/l urea, 10 g/l polypeptone, 5 g/l yeast extract, 5 g/l meat extract, 22 g/l agar, pH 6.8 with 2 M NaOH) which have been incubated at 30° C. The media are inoculated either by introducing a saline solution of C. glutamicum cells from CM plates or by adding a liquid preculture of this bacterium.

### Example 7

### In Vitro Analysis of the Function of the Proteins Encoded by the Transformed Sequences

Determination of the activities and kinetic parameters of enzymes is well known in the art. Experiments for determining the activity of a particular modified enzyme must be adapted to the specific activity of the wild-type enzyme, which is within the capabilities of the skilled worker. Reviews of enzymes in general and specific details relating to the structure, kinetics, principles, methods, applications and examples of the determination of many enzymic activities can be found for example in the following references: Dixon, M., and Webb, E. C: (1979) Enzymes, Longmans, London; Fersht (1985) Enzyme Structure and Mechanism, Freeman, New York; Walsh (1979) Enzymatic Reaction Mechanisms. Freeman, San Francisco; Price, N. C., Stevens, L. (1982) Fundamentals of Enzymology. Oxford Univ. Press: Oxford; Boyer, P. D: editor (1983) The Enzymes, 3rd edition, Academic Press, New York; Bisswanger, H. (1994) Enzymkinetik, 2nd edition, VCH, Weinheim (ISBN 3527300325); Bergmeyer, H. U., Bergmeyer, J., Graβl, M. editors (1983-1986) Methods of Enzymatic Analysis, 3rd edition, Vol. I-XII, Verlag Chemie: Weinheim; and Ullmann's Encyclopedia of Industrial Chemistry (1987) Vol. A9, “Enzymes”, VCH, Weinheim, pp. 352-363.

### Example 8

### Analysis of the Influence of the Nucleic Acids on the Production of the Amino Acids

The effect of the genetic modification in C. glutamicum on the production of an amino acid can be determined by culturing the modified microorganisms under suitable conditions (such as those described above) and investigating the medium and/or the cellular components for the increased production of the amino acid. Such analytical techniques are well known to the skilled worker and include spectroscopy, mass spectroscopy, thin-layer chromatography, staining methods of various types, enzymatic and microbiological methods, and analytical chromatography such as high performance liquid chromatography (see, for example, Ullman, Encyclopedia of Industrial Chemistry, Vol. A2, pp. 89-90 and pp. 443-613, VCH: Weinheim (1985); Fallon, A., et al., (1987) “Applications of HPLC in Biochemistry” in: Laboratory Techniques in Biochemistry and Molecular Biology, Vol. 17; Rehm et al. (1993) Biotechnology, Vol. 3, Chapter III: “Product recovery and purification”, pp. 469-714, VCH: Weinheim; Belter, P. A et al. (1988) Bioseparations: downstream processing for Biotechnology, John Wiley and Sons; Kennedy, J. F. and Cabral, J. M. S. (1992) Recovery processes for biological Materials, John Wiley and Sons; Shaeiwitz, J. A and Henry, J. D. (1988) Biochemical Separations, in Ullmann's Encyclopedia of Industrial Chemistry, Vol. B3; Chapter 11, pp. 1-27, VCH: Weinheim; and Dechow, F. J. (1989) Separation and purification techniques in biotechnology, Noyes Publications).

In addition to measurement of the final product of the fermentation, it is likewise possible to analyze other components of the metabolic pathways used to produce the desired compound, such as intermediates and byproducts, in order to determine the overall productivity of the organism, the yield and/or the efficiency of production of the compound. The analytical methods include measurements of the quantities of nutrients in the medium (e.g. sugars, hydrocarbons, nitrogen sources, phosphate and other ions), measurements of the biomass composition and of growth, analysis of the production of usual metabolites from biosynthetic pathways and measurements of gases generated during the fermentation. Standard methods for these measurements are described in Applied Microbial Physiology; A Practical Approach, P. M. Rhodes and P. F. Stanbury, editors, IRL Press, pp. 103-129; 131-163 and 165-192 (ISBN: 0199635773) and the references indicated therein.

### Example 9

### Purification of the Amino Acid from C. glutamicum Culture

The amino acid can be obtained from C. glutamicum cells and/or from the supernatant of the culture described above by various methods known in the art. For this purpose, firstly the culture supernatant is obtained, for which purpose the cells are harvested from the culture by slow centrifugation, and the cells can subsequently be fragmented or lysed by standard techniques such as mechanical force or sonication. The cell detritus is removed by centrifugation, and the supernatant fraction is taken together with the culture supernatant for further purification of the amino acid. However, it is also possible to work up the supernatant alone if the concentration of the amino acid contained in the supernatant is sufficient. The amino acid or the amino acid mixture can then be further purified by, for example, an extraction and/or salt precipitation or by an ion exchange chromatography.

If necessary and desired, further chromatography steps with a suitable resin may follow, with the amino acid either being retained on the chromatogrpahy resin, but many impurities in the sample not, or with the impurities remaining on the resin, but the sample with the product (amino acid) not. These chromatography steps may be repeated if necessary, using the same or different chromatography resins. The skilled worker is familiar with the selection of suitable chromatography resins and the most effective use for a particular molecule to be purified. The purified product can be concentrated by filtration or ultrafiltration and stored at a temperature at which the stability of the product is a maximum.

Many purification methods are known in the art and are not confined to the foregoing purification method. These are described for example in Bailey, J. E. & Ollis, D. F. Biochemical Engineering Fundamentals, McGraw-Hill: New York (1986).

The identity and purity of the isolated amino acid can be determined by standard techniques of the art. These include high performance liquid chromatography (HPLC), spectroscopic methods, staining methods, thin-layer chromatography, NIRS, enzyme assay or microbiological assays. These analytical methods are summarized in: Patek et al. (1994) Appl. Environ. Microbiol. 60: 133-140; Malakhova et al. (1996) Biotekhnologiya 11: 27-32; and Schmidt et al. (1998) Bioprocess Engineer. 19: 67-70. Ulmann's Encyclopedia of Industrial Chemistry (1996) Vol. A27, VCH: Weinheim, pp. 89-90, pp. 521-540, pp. 540-547, pp. 559-566, 575-581 and pp. 581-587; Michal, G (1999) Biochemical Pathways: An Atlas of Biochemistry and Molecular Biology, John Wiley and Sons; Fallon, A et al. (1987) Applications of HPLC in Biochemistry in: Laboratory Techniques in Biochemistry and Molecular Biology, Vol. 17.

### Example 10

### Cloning of SEQ ID NO: 1 for Expression in Plants

Unless indicated otherwise, standard methods from Sambrook et al., Molecular Cloning: A laboratory manual, Cold Spring Harbor 1989, Cold Spring Harbor Laboratory Press, are used.

The PCR amplification of SEQ ID NO: 1 took place in accordance with the protocol for Pfu Turbo DNA polymerase (from Stratagene). The composition was as follows: 1×PCR buffer [20 mM Tris-HCl (pH 8.8), 2 mM MgSO₄, 10 mM KCl, 10 mM (NH₄)SO₄, 0.1% Triton X-100, 0.1 mg/ml BSA], 0.2 mM d-Thio-dNTP and dNTP (1:125), 100 ng of genomic DNA from Saccharomyces cerevisiae (strain S288C; from Research Genetics, Inc., now Invitrogen), 50 pmol of forward primer, 50 pmol of reverse primer, 2.5 u of Pfu Turbo DNA polymerase. The amplification cycles were as follows:

1 cycle at 95° C. for 3′ followed by 36 cycles each of 1′ 95° C., 45′ 50° C., and 210″ 72° C., followed by 1 cycle at 72° C. for 8′, then 4° C.

The following primer sequences were chosen for the gene of SEQ ID NO: 1:

(SEQ ID NO:1)

i) forward primer
5′-GGAATTCCAGCTGACCACCATGACTGAATTCGAATTGCCTCCAA

(SEQ ID NO:1)

ii) reverse primer
5′-GATCCCCGGGAATTGCCATGTCAGTATTTGTAGGTTTTTATTTCGC

The first 19 nucleotides of the forward primer indicated above comprise, as universal part of the primer, cleavage sites for cloning the genes. The following part of the primer, in the indicated case 25 nucleotides, are specific for the gene to be cloned. The universal part of the reverse primer comprises at the 5′ end (20 nucleotides) again cleavage sites for the cloning. The specific part, in this 26 nucleotides, is again specific for the gene to be cloned. The universal part of the forward primer comprises and EcoRI cleavage site, whereas the universal part of the reverse primer comprises an SmaI cleavage site. Both cleavage sites were used for cloning the nucleic acid sequences. The restriction was carried out as described below. The amplicon was subsequently purified on QlAquick columns in accordance with a standard protocol (from Qiagen).

Primers for the further sequences used in the process of the invention were prepared and used analogously.

Restriction of the vector DNA (30 ng) was cut with EcoRI and SmaI by the standard protocol, and the EcoRI cleavage site was filled in by the standard protocol (MBI-Fermentas) and stopped by adding high-salt buffer. The cut vector fragments were purified on Nucleobond columns by the standard protocol (Machery-Nagel). A binary vector containing a selection cassette (promoter, selection marker, terminator) and an expression cassette with promoter, cloning cassette and terminator sequence between the T-DNA border sequences was used. The binary vector has no EcoRI and SmaI cleavage sites except in the cloning cassette. Binary vectors which can be used are known to a skilled worker, and a review of binary vectors and their use is given by Hellens, R., Mullineaux, P. and Klee H., (2000) A guide to Agrobacterium binary vectors, Trends in Plant Science, Vol. 5 No. 10, 446-451. The cloning is also advantageously possible with other restriction enzymes, depending on the vector used. Appropriate advantageous cleavage sites can be attached to the ORF by using appropriate primers for the PCR amplification.

About 30 ng of prepared vector and a defined quantity of prepared amplicon were mixed and ligated by adding ligase.

Transformation of the ligated vectors took place in the same reaction vessel by adding competent E. coli cells (strain DH5alpha) and incubating at 1° C. for 20′, followed by a heat shock at 42° C. for 90″ and cooling to 4° C. This was followed by addition of complete medium (SOC) and incubation at 37° C. for 45′. The entire mixture was then plated out on an agar plate with antibiotics (selected according to the binary vector used) and incubated at 37° C. overnight.

Successful cloning was checked by amplification using primers which bind upstream and downstream of the restriction cleavage site and thus make amplification of the insert possible. The amplification took place in accordance with the Taq DNA polymerase protocol (Gibco-BRL). The composition was as follows: 1×PCR buffer [20 mM Tris-HCL (pH 8.4), 1.5 mM MgCl₂, 50 mM KCl], 0.2 mM dNTP, 5 pmol forward primer, 5 pmol reverse primer, 0.625 u Taq DNA polymerase.

The amplification cycles were as follows: 1 cycle at 94° C. for 5′, followed by 35 cycles each of 15″ 94° C., 15″ 66° C. and 5′ 72° C., followed by 1 cycle at 72° C. for 10′, then 4° C.

Several colonies were checked, and only a colony for which a PCR product of the expected size was detected was used further.

An aliquot of this positive colony was transferred into a reaction vessel filled with complete medium (LB) and incubated at 37° C. overnight. For selection of the clone, the LB medium contained an antibiotic which was selected according to the binary vector used and the resistance gene present therein.

The plasmid preparation took place as stated in the Qiaprep standard protocol (Qiagen).

### Example 11

### Production of Transgenic Plants Expressing SEQ ID NO: 1

1 ng of the isolated plasmid DNA was transformed by electroporation into competent cells of Agrobacterium tumefaciens, for example the strain GV 3101 pMP90 (Koncz and Schell, Mol. Gen. Gent 204, 383-396, 1986). The selection of the agrobacterium strain depends on the choice of the binary vector. A review of possible strains and their properties is to be found in Hellens, R., Mullineaux, P. and Klee H., (2000) A guide to Agrobacterium binary vectors, Trends in Plant Science, Vol. 5 No. 10, 446-451. This was followed by addition of complete medium (YEP) and transfer into a new reaction vessel for 3 h at 28° C. The complete mixture was then plated out on YEP agar plates with the respective antibiotics, e.g. rifampicin and gentamycin for GV3101 pM P90, and a further antibiotic for selecting for the binary vector, and incubated at 28° C. for 48 h.

The agrobacteria with the plasmid construct generated in Example 10 were then used for plant transformation.

A colony was picked off the agar plate using a pipette tip and taken up in 3 ml of liquid TB medium which also contained appropriate antibiotics depending on the agrobacterium strain and binary plasmid. The preculture grew at 28° C. and 120 rpm for 48 h.

400 ml of LB medium which contained the same antibiotics as previously were used for the main culture. The preculture was transferred into the main culture. The latter grew at 28° C. and 120 rpm for 18 h. After centrifugation at 4000 rpm, the pellet was resuspended in infiltration medium (MS medium, 10% sucrose).

To cultivate the plants for the transformation, dishes (Piki Saat 80, green with perforated bottom, 30×20×4.5 cm, from Wiesauplast, Kunststoffltechnik, Germany) were half-filled with a GS 90 substrate (standard soil, Werkverband E.V., Germany). The dishes were watered overnight with 0.05% Previcur solution (Previcur N, Aventis CropScience or Proplant, Chimac-Agriphar, Belgium). Arabidopsis thaliana C24 seeds (Nottingham Arabidopsis Stock Centre, UK; NASC Stock N906) were scattered on the dish, about 1000 seeds per dish. The dishes were covered with a hood for the stratification (8 h, 110μ μmol/m²/s⁻¹, 22° C.; 16 h, dark, 6° C.). After 5 days, the dishes were placed in the short-day phytotron (8 h, 130 μmol/m²/s⁻¹, 22° C.; 16 h, dark, 20° C.). They remained here for about 10 days until the first true leaves were formed.

The seedlings were transferred into pots containing the same substrate (Teku pots, 7 or 10 cm, LC series, manufactured by Pöppelmann GmbH&Co, Germany). Five or nine plants were pricked out into one pot The pots were then again placed in the short-day phytotron for further growth.

After 10 days, the plants were then put in the greenhouse cubicle (additional illumination, 16 h, 340 μE, 22° C.; 8 h, dark, 20° C.). They grew here for a further 17 days.

Six-week-old, just flowering Arabidopsis plants were transformed by dipping in the suspension of agrobacteria described above for 10 sec. The latter had previously been mixed with 10 μl of Silwett L77 (Crompton S. A, Osi Specialtes, Switzerland). The corresponding method is described in Clough and Bent, 1998 (Clough, J C and Bent, A F. 1998 Floral dip: a simplified method for Agrobacterium-mediated transformation of Arabidopsis thaliana, Plant J. 16:735-743).

The plants were then laid out in a humidity chamber for 18 h. The pots were subsequently returned to the greenhouse for further growth. The plants remained there for 10 weeks until harvesting of the seeds was possible.

Depending on the resistance marker used for selecting the transformed plants, the harvested seeds were sown in a greenhouse and subjected to spray selection or else, after sterilization, cultivated on agar plates with the appropriate selecting agent After about 10-14 days, the transformed resistant plants differed distinctly from the dead wild-type seedlings and could be pricked out into 6 cm pots. The seeds of the transgenic A. thaliana plants were stored in a freezer (at −20° C.).

The other sequences used in the process were also expressed in plants analogously.

### Example 12

### Cultivation of Plants for Bioanalytical Investigations

For bioanalytical investigation of the transgenic plants they were grown uniformly in a special cultivation. For the soil mixture, the GS-90 substrate was put in a potting machine (Laible System GmbH, Singen, Germany) and used to fill pots. 35 pots were then placed together in one dish and treated with Previcur. 25 ml of Previcur were taken up in 10 l of tapwater for the treatment. This quantity was sufficient to treat about 200 pots. The pots were placed in the Previcur solution and additionally watered from above with tapwater without Previcur. The seeds were sown on the same day or within three days.

For sowing, the seeds which had been stored in the refrigerator (at −20° C.) were removed from the Eppendorf tubes using a toothpick and transferred into the pots containing the soil. In total, about 5-12 seeds were distributed in the middle of the pot.

After sowing, the dishes with the pots were covered with a matching plastic hood and placed in a stratification chamber in the dark at 4° C. for 4 days. The humidity was about 80-90%. After the stratification, the test plants were cultivated with a 16 h of light and 8 h of dark rhythm at 20° C., a humidity of 60% and a CO₂ concentration of 400 ppm for 22-23 days. The light source comprised Osram Powerstar HQI-T 250 W/D Daylight lamps which produce light of a color spectrum similar to that of the sun with a light intensity of about 220 μE/m²/s⁻¹.

The plants were subjected at an age of 8, 9 and 10 days to selection for the resistance marker. After a further 3-4 days, it was then possible clearly to differentiate the transgenic, resistant seedlings (small plants in the four-leaf stage) from the untransformed plants. The non-transgenic seedlings were bleached or dead. The transgenic resistant plants were singled out at the age of 14 days. The plants which showed the best growth in the middle of the pot were regarded as target plant. All the other plants were carefully removed with metal tweezers and discarded.

During growth, the plants were watered with distilled water from above (onto the soil) and from below into the channels. The grown plants were then harvested at an age of 23 days.

The plants having the further sequences used in the process of the invention were also analyzed analogously.

### Example 13

### Metabolic Analysis of Transformed Plants

The changes, identified according to the invention, in the contents of described metabolites were identified by the following method.

a) Sampling and Storage of Samples

Sampling took place directly in the phytotron chamber. The plants were cut with small laboratory scissors, rapidly weighed on a laboratory balance, transferred into a precooled extraction thimble and placed in an aluminum rack cooled by liquid nitrogen. If necessary, the extraction thimbles can be stored in a freezer at −80° C. The time from cutting of the plant to freezing in liquid nitrogen was not more than 10-20 s.

b) Freeze Drying

Care was taken that, during the experiment, the plants either remained in the deep-frozen state (temperatures <−40° C.) or had water removed by freeze drying before the first contact with solvents.

The aluminum rack with the plant samples in the extraction thimbles was placed in the precooled (−40° C.) freeze dryer. The initial temperature during the main drying was −35° C., and the pressure was 0.120 mbar. During the drying, the parameters were changed in accordance with a pressure and temperature program. The final temperature after 12 hours was +30° C., and the final pressure was 0.001 to 0.004 mbar. After the vacuum pump and refrigeration had been switched off, the system was ventilated with air (dried by a drying tube) or argon.

c) Extraction

The extraction thimbles with the freeze-dried plant material were transferred immediately after the ventilation of the freeze dryer into the 5 ml extraction cartridges of an ASE apparatus (Accelerated Solvent Extractor ASE 200 with Solvent Controller and AutoASE software (from DIONEX).

The 24 sample positions of the ASE apparatus were charged with plant samples.

The polar substances were extracted with about 10 ml of methanol/water (80/20, v/v) at T=70° C. and p=140 bar, 5 min heating period, 1 min static extraction. The more lipophilic substances were extracted with about 10 ml of methanol/dichloromethane (40/60, v/v) at T=70° C. and p=140 bar, 5 min heating period, 1 min static extraction. Both solvent mixtures were extracted into the same sample tubes (50 ml centrifuge tubes with screw cap and piercable septum for the ASE (DIONEX)).

The solution was mixed with internal standards: ribitol, L-glycine-2,2-d₂, L-alanine-2,3,3,3-d₄, methionine-methyl-d₃ and \overline{α}-methylglucopyranoside and methyl nonadecanoate, methyl undecanoate, methyl tridecanoate, methyl pentadecanoate, methyl nonacosanoate.

The complete extract was mixed with 8 ml of water. The solid residue of the plant sample and the extraction thimble were discarded.

The extract was shaken and then centrifuged at a minimum of 1400 g for 5 to 10 min in order to speed up phase separation. 1 ml of the supernatant methanol/water phase (“polar phase”, colorless) was removed for further GC analysis, and 1 ml was taken for LC analysis. The remainder of the methanol/water phase was discarded. 0.5 ml of the organic phase (“lipid phase”, dark green) was taken for further GC analysis, and 0.5 ml was taken for LC analysis. All the removed aliquots were evaporated to dryness using an IR Dancer infrared vacuum evaporator (Hettich). The maximum temperature during the evaporation process did not exceed 40° C. The pressure in the apparatus was not less than 10 mbar.

d) Further Processing of the Lipid Phase for LC/MS or LC/MS/MS Analysis

The lipid extract which had been evaporated to dryness was taken up in mobile phase. The HPLC run was carried out with gradient elution.

The polar extract which had been evaporated to dryness was taken up in mobile phase. The HPLC run was carried out with gradient elution.

e) Derivatization of the Lipid Phase for GC/MS Analysis

For the transmethanolysis, a mixture of 140 μl of chloroform, 37 μl of hydrochloric acid (37% by weight HCl in water), 320 μl of methanol and 20 μl of toluene was added to the evaporated extract. The vessel was tightly closed and heated at 100° C. with shaking for 2 h. The solution was then evaporated to dryness. The residue was completely dried.

The methoximation of the carbonyl groups took place by reaction with methoxyamine hydrochloride (5 mg/ml in pyridine, 100 μl in a tightly closed vessel at 60° C. for 1.5 h). 20 μl of a solution of odd-numbered, straight-chain fatty acids (0.3 mg each of fatty acids with 7 to 25 carbon atoms and 0.6 mg/ml each of fatty acids with 27, 29 and 31 carbon atoms dissolved in a mixture of 30% pyridine in toluene v/v) were added as time standards. Finally, 100 μl of N-methyl-N-(trimethylsilyl)-2,2,2-trifluoroacetamide (MSTFA) were used for derivatization in the vessel, which was again tightly closed, at 60° C. for 30 min. The final volume before GC injection was 220 μl.

f) Derivatization of the Polar Phase for GC/MS Analysis

The methoximation of the carbonyl groups took place by reaction with methoxyamine hydrochloride (5 mg/ml in pyridine, 50 μl in a tightly closed vessel at 60° C. for 1.5 h). 10 μl of a solution of odd-numbered, straight-chain fatty acids (0.3 mg each of fatty acids with 7 to 25 carbon atoms and 0.6 mg/ml each of fatty acids with 27, 29 and 31 carbon atoms dissolved in a mixture of 30% pyridine in toluene v/v) were added as time standards. Finally, 50 μl of N-methyl-N-(trimethylsilyl)-2,2,2-trifluoroacetamide (MSTFA) were used for derivatization in the vessel, which was again tightly closed, at 60° C. for 30 min. The final volume before GC injection was 110 μl.

g) Analysis of the Various Plant Samples

The plant samples were measured in single series each of 20 plant samples (so-called sequences), each sequence comprising at least 5 wild-type plants as control. The peak area or the peak height for each analyte was divided by the peak area for the respective internal standard. The data was standardized to the initial fresh weight of plant. The values calculated in this way were related to the wild-type control group by dividing them by the average of the corresponding data for the wild-type control group of the same sequence. The resulting values were referred to as x-fold, are comparable over all sequences and indicate by how much the analyte concentration differs in the mutant relative to the wild-type control.

Alternatively, the amino acids can advantageously be detected by HPLC fractionation in ethanol extracts by the method of Geigenberger et al. (Plant Cell & Environ, 19, 1996: 43-55).

The results of the various analyses of the plants are to be found in the following table:

YEL046C

| Analyte | | | | |
|---|---|---|---|---|
| No | Analyte | Ratio_by_WT | Ratio_by_median | GC/LC |
| 10000032 | Methionine | 3.46-3.58 | 3.31-3.4 | LC |
| 10000034 | Threonine | 0.45-0.15 | 0.61-0.15 | LC |
| 10000006 | Threonine | 0.17-0.16 | 0.18-0.16 | GC |
| 10000008 | Methionine | 3.31-3.67 | 3.5-3.53 | GC |

Column 1 in the table shows the sample number. The analyzed amino acid is to be found in column 2. Column 3 shows the ratio for the analyzed amino acid between the transgenic plant and the wild type. Column 4 shows the ratio for the transgenic plant compared with the median for other transgenic plants not transformed with the threonine aldolase gene. Column 5 shows the analytical method.

All the results were revealed to be significant on independent repetition of the analyses.

YJL055w

| Analyte | | | |
|---|---|---|---|
| No | Analyte | Ratio_by_WT | GC/LC |
| 10000032 | Methionine | 1.32-2.38 | LC |
| 10000034 | Threonine | 1.37-2.22 | LC 20 |
| 30000006 | Threonine | 1.19-1.89 | GC |
| 30000008 | Methionine | 1.31-2.18 | GC |

Column 1 in the table shows the analyte number. The analyzed amino acid is to be found in column 2. Column 3 shows the ratio for the analyzed amino acid between the transgenic plant and the wild type (x times according to the Ratio_by_WT method). Column 4 shows the analytical method.

All the results were revealed to be significant on independent repetition of the analyses.

1. A process for preparing amino acids selected from the group of methionine, homoserine and lysine in transgenic organisms, wherein the process comprises the following steps:
a) introduction of a nucleic acid sequence which codes for a threonine-degrading protein or lysine-degrading protein or codes for a threonine-degrading protein and lysine-degrading protein, or
b) introduction of a nucleic acid sequence which increases threonine degradation or lysine degradation or threonine degradation and lysine degradation in the transgenic organisms, and
c) expression of a nucleic acid sequence mentioned under (a) or (b) in the transgenic organism.

2. A process for preparing amino acids in transgenic organisms as claimed in claim 1, wherein the process comprises the following steps, solved:
a) introduction of a nucleic acid sequence which codes for a threonine-degrading protein which comprises the following consensus sequence
H[x]₂G[X]R[X]₁₉D[X]₇K[X]₂₇G,
or
HXDGAR[X]₃A[X]₁₅D[X]₄CXSK[X]₄PXGS[X]₃G[X]₇A[X]₄K[X]₂
GGGXRQXG
or
b) introduction of a nucleic acid sequence which increases threonine degradation in the transgenic organism, and
c) expression of a nucleic acid sequence mentioned under (a) or (b) in the transgenic organism.

3. A process for preparing amino acids in transgenic organisms as claimed in claim 1, wherein the process comprises the following steps, solved:
a) introduction of a nucleic acid sequence which codes for a lysine-degrading protein which comprises the following consensus sequence
G[X]₄GIM[X]₄₅M[X]₂RK[X]₂M[X]₁₁GGXG[X]₃E[X]₂E[X]₃W,
or
LG[X]₉LVYGG[X]₃GIMGXVA[X]₉G[X]₃GXIP[X]₂₄MHXRK[X]₂M[X]₆
F[X]₃PGGXGTXEE[X]₂E[X]₂TW[X]₂IG[X]₃KP[X]₄N[X]₃FY[X]₁₄F,
or
b) introduction of a nucleic acid sequence which increases lysine degradation in the transgenic organisms, and
c) expression of a nucleic acid sequence mentioned under (a) or (b) in the transgenic organism.

4. A process for preparing amino acids in transgenic organisms as claimed in claim 1, wherein the process comprises the following steps, solved:
a) introduction of a nucleic acid sequence which codes for a threonine-degrading protein which comprises the following consensus sequence
H[x]₂G[X]R[X]₁₉D[X]₇K[X]₂₇G,
or
HXDGAR[X]₃A[X]₁₅D[X]₄CXSK[X]₄PXGS[X]₃G[X]₇A[X]₄K[X]₂
GGGXRQXG
and introduction of a nucleic acid sequence which codes for a lysine-degrading protein which comprises the following consensus sequence
G[X]₄GIM[X]₄₅M[X]₂RK[X]₂M[X]₁₁GGXG[X]₃E[X]₂E[X]₃W,
or
LG[X]₉LVYGG[X]₃GIMGXVA[X]₉G[X]₃GXIP[X]₂₄MHXRK[X]₂M
[X]₆F[X]₃PGGXGTXEE[X]₂E[X]₂TW[X]₂IG[X]₃KP[X]₄N[X]₃FY
[X]₁₄F,
or
b) introduction of a nucleic acid sequence which codes for proteins which increase threonine degradation and lysine degradation in the transgenic organisms, and
c) expression of a nucleic acid sequence mentioned under (a) or (b) in the transgenic organism.

5. A process for preparing amino acids in transgenic organisms as claimed in claim 1, wherein there is introduction in process step (a) as set forth in claim 1 of a nucleic acid sequence which is selected from the group of nucleic acid sequences:
i) of a nucleic acid sequence having the sequence depicted in SEQ ID NO: 1, SEQ ID NO: 11; SEQ ID NO: 13; SEQ ID NO: 15; SEQ ID NO: 17; SEQ ID NO: 19; SEQ ID NO: 21; SEQ ID NO: 23 or SEQ ID NO: 25;
ii) of a nucleic acid sequence obtained owing to the degeneracy of the genetic code through back-translation of the amino acid sequence depicted in SEQ ID NO: 2, SEQ ID NO: 12; SEQ ID NO: 14; SEQ ID NO: 16; SEQ ID NO: 18; SEQ ID NO: 20; SEQ ID NO: 22; SEQ ID NO: 24 or SEQ ID NO: 26, and
iii) of a derivative of the nucleic acid sequence depicted in SEQ ID NO: 1, SEQ ID NO: 11; SEQ ID NO: 13; SEQ ID NO: 15; SEQ ID NO: 17; SEQ ID NO: 19; SEQ ID NO: 21; SEQ ID NO: 23 or SEQ ID NO: 25; which codes for polypeptides having the amino acid sequence depicted in SEQ ID NO: 2, SEQ ID NO: 12; SEQ ID NO: 14; SEQ ID NO: 16; SEQ ID NO: 18; SEQ ID NO: 20; SEQ ID NO: 22; SEQ ID NO: 24 or SEQ ID NO: 26 and have at least 50% homology at the amino acid level, with a negligible reduction in the biological activity of the polypeptides.

6. A process for preparing amino acids in transgenic organisms as claimed in claim 1, wherein there is introduction in process step (a) of a nucleic acid sequence which is selected from the group of nucleic acid sequences:
i) of a nucleic acid sequence obtained owing to the degeneracy of the genetic code through back-translation of the amino acid sequence depicted in SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10;
ii) of a derivative of the nucleic acid sequence which is obtained by back-translation of the amino acid sequence depicted in SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 and which has at least 70% homology at the amino acid level with the aforementioned amino acid sequences, with a negligible reduction in the biological activity of the polypeptides.

7. A process for preparing amino acids in transgenic organisms as claimed in claim 1, wherein the transgenic organism is cultivated and harvested after introduction and expression of the nucleic acid.

8. A process for preparing amino acids in transgenic organisms as claimed in claim 1, wherein the amino acid is isolated from the organism or the culture medium or the organism and the culture medium.

9. A process for preparing amino acids in transgenic organisms as claimed in claim 1, wherein the essential amino acid methionine is involved.

10. A process for preparing amino acids in transgenic organisms as claimed in claim 1, wherein the transgenic organism is a microorganism or a plant.

11. A process for preparing amino acids in transgenic organisms as claimed in claim 10, wherein the transgenic organism is a microorganism selected from the group of genera Corynebacterium, Brevibacterium, Escherichia, Bacillus, Rhodotorula, Hansenula, Schizosaccharomyces, Saccharomyces, Candida, Claviceps or Flavobacterium.

12. A process for preparing amino acids in transgenic organisms as claimed in claim 10, wherein the transgenic organism is a plant selected from the group of crop plants.

13. A process for preparing amino acids in transgenic organisms as claimed in claim 12, wherein the transgenic organism is a plant selected from the group of peanut, oilseed rape, canola, sunflower, safflower, olive, sesame, hazelnut, almond, avocado, bay, pumpkin, lettuce, flax, soybean, pistachio, borage, corn, wheat, rye, oats, millet, triticale, rice, barley, cassaya, potato, sugar beet, feed beet, aubergine, tomato, pea, alfalfa and perennial grasses and feed crops.

14. A process for preparing amino acids in transgenic organisms as claimed in claim 1, wherein the nucleic acid sequence is derived from a eukaryote.

15. A process for preparing amino acids in transgenic organisms as claimed in claim 1, wherein the nucleic acid sequence is derived from the genus Saccharomyces.

16. A process for preparing amino acids in transgenic organisms as claimed in claim 1, wherein the nucleic acid sequence is for introduction and for expression incorporated into a nucleic acid construct or a vector.

17. A process for preparing amino acids in transgenic organisms as claimed in claim 1, wherein additionally biosynthesis genes of the amino acid prepared in the process are introduced into the organism.

18. A nucleic acid construct comprising a nucleic acid sequence selected from the group consisting of:
a) a nucleic acid which codes for a threonine-degrading protein which comprises the following consensus sequence H[x]₂G[X]R[X]₁₉ D[X]₇K[X]₂₇G, or HXDGAR[X]₃A[X]₁₅D[X]₄CXSK[X]₄PXGS[X]₃G[X]₇A[X]₄K[X]₂GGGXRQXG
b) a nucleic acid sequence which codes for a lysine-degrading protein which comprises the following consensus sequence
G[X]₄GIM[X]₄₅M[X]₂RK[X]₂M[X]₁₁GGXG[X]₃E[X]₂E[X]₃W,
or
LG[X]₉LVYGG[X]₃GIMGXVA[X]₉G[X]₃GXIP[X]₂₄MHXRK[X]₂M[X]₆F[X]₃PGGXGT
XEE[X]₂E[X]₂TW[X]₂IG[X]₃KP[X]₄N[X]₃FY[X]₁₄F;
c) a nucleic acid sequence which comprises the nucleic acid sequence under (a) and the nucleic acid sequence under (b);
d) a nucleic acid sequence having the sequence depicted in SEQ ID NO: 1, SEQ ID NO: 1:SEQ ID NO: 13:SEQ ID NO: 15:SEQ ID NO: 17:SEQ ID NO: 19; SEQ ID NO: 21; SEQ ID NO: 23 or SEQ ID NO: 25;
e) a nucleic acid sequence obtained owing to the degeneracy of the genetic code through back-translation of the amino acid sequence depicted in SEQ ID NO: 2. SEQ ID NO: 12: SEQ ID NO: 14; SEQ ID NO: 16; SEQ ID NO: 18; SEQ ID NO: 20; SEQ ID NO: 22; SEQ ID NO: 24 or SEQ ID NO: 26;
f) a derivative of the nucleic acid sequence depicted in SEQ ID NO: 1, SEQ ID NO: 11; SEQ ID NO: 13; SEQ ID NO: 15; SEQ ID NO: 17: SEQ ID NO: 19: SEQ ID NO: 21; SEQ ID NO: 23 or SEQ ID NO: 25; which codes for polypeptides having the amino acid sequence depicted in SEQ ID NO: 2. SEQ ID NO: 12; SEQ ID NO: 14; SEQ ID NO: 16; SEQ ID NO: 18; SEQ ID NO: 20: SEQ ID NO: 22; SEQ ID NO: 24 or SEQ ID NO: 26 and have at least 50% homology at the amino acid level, with a negligible reduction in the biological activity of the polypeptides;
g) a nucleic acid sequence obtained owing to the degeneracy of the genetic code through back-translation of the amino acid sequence depicted in SEQ ID NO: 3, SEQ ID NO: 4, SEQ ID NO: 5. SEQ ID NO: 6, SEQ ID NO: 7, SEQ ID NO: 8. SEQ ID NO: 9 or SEQ ID NO: 10; and
h) a derivative of the nucleic acid sequence which is obtained by back-translation of the amino acid sequence depicted in SEQ ID NO: 3, SEQ ID NO: 4, SEQ ID NO: 5, SEQ ID NO: 6, SEQ ID NO: 7. SEQ ID NO: 8. SEQ ID NO: 9 or SEQ ID NO: 10 and which has at least 70% homology at the amino acid level with the aforementioned amino acid sequences, with a negligible reduction in the biological activity of the polypeptides;
wherein the nucleic acid sequence is functionally linked to one or more regulatory signals.

19. A vector comprising a nucleic acid sequence selected from the group consisting of:
a) a nucleic acid which codes for a threonine-degrading protein which comprises the following consensus sequence H[x]₂G[X]R[X]₁₉D[X]₇K[X]₂₇G, or HXDGAR[X]₃A[X]₁₅[X]₄CXSK[X]₄PXGS[X]₃G[X]₇A[X]₄K[X]₂GGGXRQXG
b) a nucleic acid sequence which codes for a lysine-degrading protein which comprises the following consensus sequence
G[X]₄GIM[X]₄₅M[X]₂RK[X]₂M[X]₁₁GGXG[X]₃E[X]₂E[X]₃W,
or
LG[X]₉LVYGG[X]₃GIMGXVA[X]₉G[X]₃GXIP[X]₂₄MHXRK[X]₂
M[X]₆F[X]₃PGGXGTXEE[X]₂E[X]₂TW[X]₂IG[X]₃KP[X]₄N[X]₃
FY[X]₁₄F;
c) a nucleic acid sequence which comprises the nucleic acid sequence under (a) and the nucleic acid sequence under (b);
d) a nucleic acid sequence having the sequence depicted in SEQ ID NO: 1. SEQ ID NO: 11; SEQ ID NO: 13; SEQ ID NO: 15; SEQ ID NO: 17; SEQ ID NO: 19, SEQ ID NO: 21; SEQ ID NO: 23 or SEQ ID NO: 25;
e) a nucleic acid sequence obtained owing to the degeneracy of the genetic code through back-translation of the amino acid sequence depicted in SEQ ID NO: 2, SEQ ID NO: 12; SEQ ID NO: 14; SEQ ID NO: 16; SEQ ID NO: 18; SEQ ID NO: 20; SEQ ID NO: 22; SEQ ID NO: 24 or SEQ ID NO: 26;
f) a derivative of the nucleic acid sequence depicted in SEQ ID NO: 1, SEQ ID NO: 11; SEQ ID NO: 13; SEQ ID NO: 15; SEQ ID NO: 17; SEQ ID NO: 19; SEQ ID NO: 21; SEQ ID NO: 23 or SEQ ID NO: 25; which codes for polypeptides having the amino acid sequence depicted in SEQ ID NO: 2, SEQ ID NO: 12; SEQ ID NO: 14; SEQ ID NO: 16; SEQ ID NO: 18; SEQ ID NO: 20; SEQ ID NO: 22; SEQ ID NO: 24 or SEQ ID NO: 26 and have at least 50% homology at the amino acid level, with a negligible reduction in the biological activity of the polypeptides;
g) a nucleic acid sequence obtained owing to the degeneracy of the genetic code through back-translation of the amino acid sequence depicted in SEQ ID NO: 3, SEQ ID NO: 4. SEQ ID NO: 5. SEQ ID NO: 6. SEQ ID NO: 7, SEQ ID NO: 8, SEQ ID NO: 9 or SEQ ID NO: 10; and
h) a derivative of the nucleic acid sequence which is obtained by back-translation of the amino acid sequence depicted in SEQ ID NO: 3. SEQ ID NO: 4, SEQ ID NO: 5, SEQ ID NO: 6, SEQ ID NO: 7. SEQ ID NO: 8. SEQ ID NO: 9 or SEQ ID NO: 10 and which has at least 70% homology at the amino acid level with the aforementioned amino acid sequences, with a negligible reduction in the biological activity of the polypeptides.

20. A transgenic prokaryotic or eukaryotic organism comprising at least one nucleic acid sequence selected from the group consisting of:
a) a nucleic acid which codes for a threonine-degrading protein which comprises the following consensus sequence H[x]₂G[X]R[X]₁₉D[X]₇K[X]₂₇G, or HXDGAR[X]₃A[X]₁₅D[X]₄CXSK[X]₄PXGS[X]₃G[X]₇A[X]₄K[X]₂GGGXRQXG;
b) a nucleic acid sequence which codes for a lysine-degrading protein which comprises the following consensus sequence
G[X]₄GIM[X]₄₅M[X]₂RK[X]₂M[X]₁₁GGXG[X]₃E[X]₂E[X]₃W,
or
LG[X]₉LVYGG[X]₃GIMGXVA[X]₉G[X]₃GXIP[X]₂₄MHXRK[X]₂
M[X]₆F[X]₃PGGXGTXEE[X]₂E[X]₂TW[X]₂IG[X]₃KP[X]₄N[X]₃
FY[X]₁₄F;
c) a nucleic acid sequence which comprises the nucleic acid sequence under (a) and the nucleic acid sequence under (b);
d) a nucleic acid sequence having the sequence depicted in SEQ ID NO: 1, SEQ ID NO: 11: SEQ ID NO: 13: SEQ ID NO: 15; SEQ ID NO: 17; SEQ ID NO: 19; SEQ ID NO: 21; SEQ ID NO: 23 or SEQ ID NO: 25;
e) a nucleic acid sequence obtained owing to the degeneracy of the genetic code through back-translation of the amino acid sequence depicted in SEQ ID NO: 2, SEQ ID NO: 12; SEQ ID NO: 14; SEQ ID NO: 16; SEQ ID NO: 18; SEQ ID NO: 20; SEQ ID NO: 22; SEQ ID NO: 24 or SEQ ID NO: 26;
f) a derivative of the nucleic acid sequence depicted in SEQ ID NO: 1. SEQ ID NO: 11; SEQ ID NO: 13; SEQ ID NO: 15; SEQ ID NO: 17; SEQ ID NO: 19; SEQ ID NO: 21; SEQ ID NO: 23 or SEQ ID NO: 25; which codes for polypeptides having the amino acid sequence depicted in SEQ ID NO: 2, SEQ ID NO: 12; SEQ ID NO: 14; SEQ ID NO: 16; SEQ ID NO: 18; SEQ ID NO: 20; SEQ ID NO: 22; SEQ ID NO: 24 or SEQ ID NO: 26 and have at least 50% homology at the amino acid level, with a negligible reduction in the biological activity of the polypeptides;
g) a nucleic acid sequence obtained owing to the degeneracy of the genetic code through back-translation of the amino acid sequence depicted in SEQ ID NO: 3, SEQ ID NO: 4, SEQ ID NO: 5. SEQ ID NO: 6, SEQ ID NO: 7. SEQ ID NO: 8, SEQ ID NO: 9 or SEQ ID NO: 10; and
h) a derivative of the nucleic acid sequence which is obtained by back-translation of the amino acid sequence depicted in SEQ ID NO: 3. SEQ ID NO: 4. SEQ ID NO: 5, SEQ ID NO: 6. SEQ ID NO: 7, SEQ ID NO: 8. SEQ ID NO: 9 or SEQ ID NO: 10 and which has at least 70% homology at the amino acid level with the aforementioned amino acid sequences, with a negligible reduction in the biological activity of the polypeptides.

21. A transgenic prokaryotic or eukaryotic organism as claimed in claim 20, which is a microorganism or a plant.

22. A transgenic prokaryotic or eukaryotic organism as claimed in claim 21, which is a microorganism of the genus Corynebacterium or Brevibacterium.

23. A transgenic prokaryotic or eukaryotic organism as claimed in claim 21, which is a plant selected from the group of genus of peanut, oilseed rape, canola, sunflower, safflower, olive, sesame, hazelnut, almond, avocado, bay, pumpkin, lettuce, flax, soybean, pistachio, borage, corn, wheat, rye, oats, millet, triticale, rice, barley, cassaya, potato, sugar beet, feed beet, aubergine, tomato, pea, alfalfa and perennial grasses and feed crops.

24. A process for producing an animal or human food, for producing cosmetics or pharmaceuticals, comprising growing or culturing the transgenic organism of claim 20, recovering the amino acid produced, and formulating the amino acid into an animal or human food, cosmetics or pharmaceuticals.

25. An amino acid sequence selected from the group of sequences SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.


SEQUENCE LISTING (26 sequences)

SEQ ID NO: 1 - DNA, 1164 bp, Saccharomyces cerevisiae
  CDS (1)..(1164): Threonine aldolase
atgactgaattcgaattgcctccaaaatatatcaccgctgctaacgacttgcggtcagacacattcaccactccaactgcagagatgatggaggccgctttagaggcctctatcggtgacgctgtctacggtgaagatgttgacaccgttaggctcgaacagaccgttgcccgcatggctggcaaagaagcaggtttgttctgtgtctctgggactttgtccaaccagattgccatcagaactcacttgatgcaacctccatactctattctatgtgattacagggctcacgtttacactcacgaagccgctggactggcgatcttgtctcaagcgatggtggttcctgtggttccttccaacggtgactacttgaccttggaagacatcaagtcacactacgtcccagacgacggtgatattcacggtgcccccaccagattgatttctctggaaaacactttacacggtattgtttatccattggaagaactggtccgcatcaaagcttggtgtatggaaaatggtctcaaactacattgtgacggtgccagaatctggaatgccgctgcacaatctggcgtgccattaaagcaatatggggaaatcttcgactccatctccatctgtctatccaagtctatgggtgctcctattgggtccgtcttggttgggaaccttaagtttgtcaagaaggccacccatttcagaaaacaacaaggtggtggtattagacaatctggtatgatggctagaatggctcttgtaaacatcaacaacgattggaagtcccaattgctgtactcgcactctttggctcatgaattagccgaatattgtgaggcaaagggcatcccgctagagtctccagcagacaccaactttgtctttattaacctgaaggccgctagaatggacccagatgtccttgttaagaagggtttgaagtacaacgttaagctaatgggtggtagagtctcgttccactatcaagtcaccagagatactttggaaaaagtcaaattggccatctccgaggccttcgactatgctaaagaacatcctttcgactgtaacggacctacccagatttaccgtagtgaatccaccgaggtcgacgttgatggcaacgctatccgcgaaataaaaacctacaaatactga

SEQ ID NO: 2 - protein, 387 aa, Saccharomyces cerevisiae
MTEFELPPKYITAANDLRSDTFTTPTAEMMEAALEASIGDAVYGEDVDTVRLEQTVARMAGKEAGLFCVSGTLSNQIAIRTHLMQPPYSILCDYRAHVYTHEAAGLAILSQAMVVPVVPSNGDYLTLEDIKSHYVPDDGDIHGAPTRLISLENTLHGIVYPLEELVRIKAWCMENGLKLHCDGARIWNAAAQSGVPLKQYGEIFDSISICLSKSMGAPIGSVLVGNLKFVKKATHFRKQQGGGIRQSGMMARMALVNINNDWKSQLLYSHSLAHELAEYCEAKGIPLESPADTNFVFINLKAARMDPDVLVKKGLKYNVKLMGGRVSFHYQVTRDTLEKVKLAISEAFDYAKEHPFDCNGPTQIYRSESTEVDVDGNAIREIKTYKY

SEQ ID NO: 3 - protein, 376 aa, Canola
GCFACYLVGGFSVQEKMVTRIVDLRSDTVTKPTEAMRAAMASAEVDDDVLGYDPTAFRLETEMAKTMGKEAALFVPSGTMGNLVSVLVHCDVRGSEVILGDNCHINIFENGGIATIGGVHPRQVKNNDDGTMDIDLIEAAIRDPMGELFYPTTKLICLENTHANSGGRCLSVEYTDRVGELAKKHGLKLHIDGARIFNASVALGVPVDRLVQAADSVSVCLSKGIGAPVGSVIVGSKNFIAKARRLRKTLGGGMRQIGLLCAAALVALQENVGKLESDHKKARLLADGLNEVKGLRVDACSVETNMVFIDIEEGTKTRAEKICKYMEERGILVMQESSSRMRVVLHHQISASDVQYALSCFQQALAVKGVQKEMGN

SEQ ID NO: 4 - protein, 115 aa, Soybean
LFGLLAILLEYLEKMVPRIVDLRSDTVTKPSEAMRAAMASAEVDDDVLGRDPSCFRLETEMAKILGKEGALFVPSGTMANLISVLVHCDIRGSEVILGDNSHIHIYENGGIATLG

SEQ ID NO: 5 - protein, 127 aa, Rice
  misc_feature (1)..(127): unknown or other
KTLXGGMRQVGILCAAALVALQENVGKLQSDHNKAKLLADGLNEIKGLRVDISSVETNIIYVEVEEGSRATAAKLCKDLEDYGILLMPMGSSRLRIVFHHQISASDVQYALSCFQQAVNGVRNENGN

SEQ ID NO: 6 - protein, 147 aa, Rice
GRRFRAIRDPMGELFYPTTKLICLENTHANSGGRCLSVEYTDRVGELAKKHGLKLHIDGARIFNASVALGVPVDRLVQAADSVSVCLSKGIGAPVGSVIVGSKNFIAKARRLRKTLGGGMRQIGLLCAAALVALQENVGKLESDHKK

SEQ ID NO: 7 - protein, 169 aa, Canola
  misc_feature (1)..(169): unknown or other
GIPGXTFRGDVAKSHGLKLHIDGARIFNASVALGVPVHRLVKAADSVSVCISKGLGAPVGSVIVGSTAFIEKAKILTKTLGGGMRQVGILCAAAYVAVRDTVGKLADDHRRAKVLADGLKKIKHFRVDTTSVETNMVFFDIVDSRISPDKLCQVLEQRNVLAMPAGSKR

SEQ ID NO: 8 - protein, 362 aa, Canola
IEIKMVMRTVDLRSDTVTRPTDAMREAMGSAEVDDDVLGYDPTARRLEEEIAKMMGKEAALFVPSGTMGNLICVMVHCDVRGSEVILGDNCHIHVYENGGISTIGGVHPKTIKNEEDGTMDLGAIEAAIRDPKGSTFYPSTRLICLENTHANSGGRCLSAEYTDRVGEIAKRHGLKLHIDGARLFNASIALGVPVHRLVQAADSVSVCLSKGLGAPIGSVVVGSQSFIEKAKTLRKTLGGGMRQIGVLCAAALVALQENLPKLQFDHKKTKLLAEGLNQMKGIRVNVAAMETNMIFMDMEDGSKLTAEKLRKSLTEHGILVIPENSTRIRMVLHHQITTSDVHYTLSCLQQAVQTIHEPCQN

SEQ ID NO: 9 - protein, 196 aa, Canola
GFLLKHKYIYYCCYLFESKSNNFLFSVIKMVTPVIRTVDLRSDTVTKPTESMRSAMANAEVDDDVLGNDPTAVLLEREVAEIAGKEAAMFVPSGTMGNLISVLVHCDERGSEVILGDDSHIHIYENGGVSSLGGVHPRTVKNEEDGTMEISSIEAAVRSPTGDLHYPVTKLICLENTQANCGGRCLPIEYIDKVGE

SEQ ID NO: 10 - protein, 104 aa, Soybean
IGIKMVMRIVDLRSDTVTRPTDAMREAMASAEVDDDVLGYDPTARGLEEEMAKMMGKEAALFVPSGTMGNLICVMVHCDVRGSEVILGDTCHIHVYENGGISTI

SEQ ID NO: 11 - DNA, 738 bp, Saccharomyces cerevisiae
  CDS (1)..(738): Protein similar to lysine decarboxylase
atgacaatggaaaaaaatggaggtaatagcagccgtggtggccaagtaggcggcaagtctgtgtgtgtttactgcgggtcttcatttggcgctaaggcgctatactcagaaagtgcagaagaattaggagcccttttccataagctgggatggaaattggtatacggtggaggcactactggtttgatgggcaagatagcaaggtctacgatgggacctgatttaagcggacaggttcacggtatcattccaaatgcacttgtgtctaaggaaaggacagacgaggataaagaagatgttaataaagcattgttggagtctgtagaaaatcataagggcgccactcctatttctgaagagtatggggaaacaacgattgtaccagatatgcatacgagaaaaagaatgatggcaaatttgagtgacgcgtttgttgctatgcctggtggatacgggacttttgaagaaatcatggaatgtatcacgtggtcgcaactggggattcataataaaccaattatcttgttcaatatcgatgggttctatgacaaattattggagttcctcaaacactctattcaagaacggttcatcagtgtgaagaatggtgaaatcattcaagttgcctccactccgcaggaagttgttgataaaatagagaagtacgtcgttccagagggccgtttcaatttgaattggagcgacgaaggtcacgctcacgaggattgtgctaaataa

SEQ ID NO: 12 - protein, 245 aa, Saccharomyces cerevisiae
MTMEKNGGNSSRGGQVGGKSVCVYCGSSFGAKALYSESAEELGALFHKLGWKLVYGGGTTGLMGKIARSTMGPDLSGQVHGIIPNALVSKERTDEDKEDVNKALLESVENHKGATPISEEYGETTIVPDMHTRKRMMANLSDAFVAMPGGYGTFEEIMECITWSQLGIHNKPIILFNIDGFYDKLLEFLKHSIQERFISVKNGEIIQVASTPQEVVDKIEKYVVPEGRFNLNWSDEGHAHEDCAK

SEQ ID NO: 13 - DNA, 1083 bp, Glycine max
  CDS (1)..(1083): Threonine aldolase
atggtaactagaattgtggatcttcggtcagacacagttacaaagccaactgaagcaatgagagctgctatggcaagtgctgaagttgatgacgatgttctaggctatgatccaactgcttttcgcttagaaacagagatggcaaagacaatgggcaaagaagctgctctttttgttccatctggcactatggggaaccttgtatctgtacttgttcattgtgatgtcaggggaagtgaggttattcttggagacaattgccatatcaacatttttgagaatggaggcattgcaaccattgggggagtgcatccaagacaagtgaaaaataacgatgatggaaccatggacattgatttgattgaggctgctatcagggacccaatgggggagctattctatccaaccaccaagcttatttgcttggaaaatactcatgcaaactctggtggcagatgcctctcagttgaatatacagacagagttggagagttagctaagaagcatggactgaagcttcacattgatggggcccgtatttttaacgcatcagttgcacttggtgttccagtggataggcttgtccaggcggctgattcagtttccgtttgcctatctaaaggtataggtgctccagttggatctgttattgttggttccaagaattttattgccaaggctagacgactccggaaaaccttaggaggtggaatgagacagattggcctcctttgtgccgctgcacttgttgccttgcaggaaaatgttgggaagctggaaagtgatcacaagaaagctagacttttggctgatggattaaacgaagttaaaggattgagagtggatgcctgttctgtggagaccaatatggtatttattgacattgaagagggtacaaagactagagcagaaaagatatgcaagtacatggaagaacgtggtatccttgtgatgcaagagagttcatcaagaatgagagttgttctccatcaccaaatatcagcaagtgatgtgcaatatgccttgtcgtgctttcagcaagctctagctgtcaaaggagtacaaaaggaaatgggcaactaa

SEQ ID NO: 14 - protein, 360 aa, Glycine max
MVTRIVDLRSDTVTKPTEAMRAAMASAEVDDDVLGYDPTAFRLETEMAKTMGKEAALFVPSGTMGNLVSVLVHCDVRGSEVILGDNCHINIFENGGIATIGGVHPRQVKNNDDGTMDIDLIEAAIRDPMGELFYPTTKLICLENTHANSGGRCLSVEYTDRVGELAKKHGLKLHIDGARIFNASVALGVPVDRLVQAADSVSVCLSKGIGAPVGSVIVGSKNFIAKARRLRKTLGGGMRQIGLLCAAALVALQENVGKLESDHKKARLLADGLNEVKGLRVDACSVETNMVFIDIEEGTKTRAEKICKYMEERGILVMQESSSRMRVVLHHQISASDVQYALSCFQQALAVKGVQKEMGN

SEQ ID NO: 15 - DNA, 1077 bp, Brassica napus
  CDS (1)..(1077): Threonine aldolase
atggtgatgcgaactgtggatctacggtcagacaccgtgactagacctaccgatgccatgcgtgaagcaatgggaagcgcagaagtagacgatgacgtcctcggctacgacccaacggctcgacgtcttgaagaggagatagccaagatgatggggaaagaagcagctctcttcgtgccatctggtacaatggggaacctcatatgcgttatggttcactgcgacgtgagaggcagcgaggtgattcttggagacaactgtcacatccatgtctacgagaacggagggatatcaacgataggaggcgtgcatcccaagacaatcaagaatgaagaagacgggacaatggacttgggggctatagaagcagctattagagatcctaaaggaagcacgttttatccatcaacaaggttgatttgtttggagaacacacatgccaactctggtgggagatgtttgagtgcggaatacacagatagagttggagagattgccaagagacatggattaaagcttcatatcgatggagctcgcctttttaatgcttccattgcacttggagttccagtccataggcttgtacaggctgctgactctgtttcggtgtgtctctctaaaggtcttggagctccaataggatctgtagtcgttggttcacagagtttcatagaaaaggcgaaaacgttaagaaaaacattaggtggaggaatgagacaaataggcgtcctgtgcgcagccgctttggtcgcacttcaagagaatctcccaaagttacaatttgaccacaagaagacaaaattgttagctgaagggttgaatcaaatgaaagggattagagtgaacgttgcagccatggagaccaacatgatattcatggatatggaggatggatcaaaactgaccgctgaaaaactccgcaagagtctaacggagcatggcattctcgtcatccctgaaaactctacccgaatcagaatggttctacaccaccagataacaacaagtgatgtgcattacacattgtcttgcttacaacaagcagtgcagacgattcatgaaccatgccaaaactaa

SEQ ID NO: 16 - protein, 358 aa, Brassica napus
MVMRTVDLRSDTVTRPTDAMREAMGSAEVDDDVLGYDPTARRLEEEIAKMMGKEAALFVPSGTMGNLICVMVHCDVRGSEVILGDNCHIHVYENGGISTIGGVHPKTIKNEEDGTMDLGAIEAAIRDPKGSTFYPSTRLICLENTHANSGGRCLSAEYTDRVGEIAKRHGLKLHIDGARLFNASIALGVPVHRLVQAADSVSVCLSKGLGAPIGSVVVGSQSFIEKAKTLRKTLGGGMRQIGVLCAAALVALQENLPKLQFDHKKTKLLAEGLNQMKGIRVNVAAMETNMIFMDMEDGSKLTAEKLRKSLTEHGILVIPENSTRIRMVLHHQITTSDVHYTLSCLQQAVQTIHEPCQN

SEQ ID NO: 17 - DNA, 570 bp, Glycine max
  CDS (1)..(570): Lysine decarboxylase
atggaaataagggtttcaaagttcaagaggatttgtgtcttctgtgggagtagccctggcaaaaagagaagctaccaagatgctgccattgaacttggcaatgaattggtctcaaggaacattgatctggtgtatggagggggaagcattggtctaatgggtttagtttcacaagctgttcatgatggcggtcggcatgtcatcggagttattcccaagaccctcatgcctcgagagctaactggtgaaacagtgggagaagtaaaagctgttgctgatatgcaccaaaggaaggcagagatggccaagcattcagacgcctttattgccttaccaggtggatatgggactctagaggagcttcttgaagtcataacctgggcacaacttgggattcatgacaagccggtgggattagtaaatgttgatggatactttaattccttgctgtcatttattgacaaagctgtggaagagggatttatcagtccaaatgctcgccacataattgtatcagcacccacagcaaaagagttggtgaagaaattggaggattacgttccctgttaa

SEQ ID NO: 18 - protein, 189 aa, Glycine max
MEIRVSKFKRICVFCGSSPGKKRSYQDAAIELGNELVSRNIDLVYGGGSIGLMGLVSQAVHDGGRHVIGVIPKTLMPRELTGETVGEVKAVADMHQRKAEMAKHSDAFIALPGGYGTLEELLEVITWAQLGIHDKPVGLVNVDGYFNSLLSFIDKAVEEGFISPNARHIIVSAPTAKELVKKLEDYVPC

SEQ ID NO: 19 - DNA, 675 bp, Hordeum vulgare
  CDS (1)..(675): Lysine decarboxylase
atgggcgacaccaccgcgccctcgccgccgaggaggttcggcaggatctgcgtcttctgcggcaggaactccggcaaccgcgccgtgttcggcgacgccgcgctcgagctcggccagggcctggtgacgaggggggtcgatctggtctacggcggcggcagtatcgggctgatgggcctgatcgcgcagacggttctcgacggcggctgccgcgtcctcggggtgattccaagagcactcatgcccctcgagatatccggtgcaagtgttggagaagtaaagattgtctccgacatgcatgagaggaaagctgagatggcgcgacaagccgatgcattcattgctcttccgggtgggtatggaacaatggaagagctggtagagatgatcacttggtcgcagcttggaatccatgacaaaccggtcgggttgctaaacgtcgatgggtactatgatccgttactcgcgctgttcgacaagggcgcgggggaagggttttttaaggccgattgcaggccgataatcgtgtcggcaccaactgcccacgaactgctgacaaaaatggagcaatacacccgttcaccccgggaggtggcctcgcggacgagctgggagatgaccgagatgggctccgggaaagcaccggagccggaggaggaggcggcggcatcgtaa

SEQ ID NO: 20 - protein, 224 aa, Hordeum vulgare
MGDTTAPSPPRRFGRICVFCGRNSGNRAVFGDAALELGQGLVTRGVDLVYGGGSIGLMGLIAQTVLDGGCRVLGVIPRALMPLEISGASVGEVKIVSDMHERKAEMARQADAFIALPGGYGTMEELVEMITWSQLGIHDKPVGLLNVDGYYDPLLALFDKGAGEGFFKADCRPIIVSAPTAHELLTKMEQYTRSPREVASRTSWEMTEMGSGKAPEPEEEAAAS

SEQ ID NO: 21 - DNA, 717 bp, Artificial Sequence
  CDS (1)..(717): Lysine decarboxylase
atggaggagaatcaagagaagtttgctccggagagcagcggcggcgacggtggtggctcggtgagaacgatctgcgtcttctgcggcagcaggccggggaaccggccgtccttcagcgctgcggcgctcgacctggggaagcagctggtcgagaggcagatgaacctggtgtacggcggcggcagcggcgggctgatgggcctggtgtccaaggccgtctacgaaggcggccgccacgtcctcggggtcatccctaccgccctcctacctgaagaggtgtcaggggagacattgggagaggtgaaagtggtcagggacatgcatcagcgcaaggcggaaatggcgaaacatgccgacgctttcatcgccctgccaggtggttacgggacaatcgaagaactgctggagatcatagcgtgggcgcagctgggcatccacagcaaaccggtggggttgctcaacgtggacggctactacaacagcctgctctcgctgttcgacaaggctgtcgaggagggcttcatcgacaccaaggcacggaacatcttcgtcctcgctgacaccgccgccgacctgctgactaggctcaccatgatggcgcgcctggcagccgacgacgacgatgctactactacccccagaggagacggagacggagacggagacgaacacaagggggccaccaccgctgcaggcgtcaaaaggaaaaggggctaa

SEQ ID NO: 22 - protein, 238 aa, Artificial Sequence
  Artificial
MEENQEKFAPESSGGDGGGSVRTICVFCGSRPGNRPSFSAAALDLGKQLVERQMNLVYGGGSGGLMGLVSKAVYEGGRHVLGVIPTALLPEEVSGETLGEVKVVRDMHQRKAEMAKHADAFIALPGGYGTIEELLEIIAWAQLGIHSKPVGLLNVDGYYNSLLSLFDKAVEEGFIDTKARNIFVLADTAADLLTRLTMMARLAADDDDATTTPRGDGDGDGDEHKGATTAAGVKRKRG

SEQ ID NO: 23 - DNA, 717 bp, Zea mays; identical to SEQ ID NO: 21
  CDS (1)..(717): Lysine decarboxylase

SEQ ID NO: 24 - protein, 238 aa, Zea mays; identical to SEQ ID NO: 22

SEQ ID NO: 25 - DNA, 672 bp, Oryza sativa
  CDS (1)..(672): Lysine decarboxylase
atgggcgacaacagcgccgccgcggcggccgtggccgcgccgcgcggcaggttcggcaggatctgcgtcttctgcggcagcaacgccggcaaccgcgcggtgttcggcgacgcggcgctccagctcgggcaggagctggtgtcgagagggatcgagttggtctacggtggcggcagcgtcgggttgatgggcttgatcgcgcagacggttcttgatggcggctgcggtgttctcggggtgattccaaaagcacttatgcccaccgagatatcaggtgcaagtgttggagaagtgaaaattgtgtctgacatgcatgagaggaaagctgagatggcacgccaatccgatgccttcatcgctcttcctggagggtatggaacaatggaggagttgttagagatgataacttggtcacaacttggaattcatgacaaaccagttgggttgctgaatgtggacggttactatgatccgttgcttgcgctatttgataagggtgcggcagaaggatttattaaggccgattgcagacaaataattgtttcggcaccgactgcgcatgagctgctgagaaagatggagcaatacactcgttcacaccaggaggtagcgccacgtacaagctgggagatgtcagagcttggttatggaaagacaccagaggaatcgtaa

SEQ ID NO: 26 - protein, 223 aa, Oryza sativa
MGDNSAAAAAVAAPRGRFGRICVFCGSNAGNRAVFGDAALQLGQELVSRGIELVYGGGSVGLMGLIAQTVLDGGCGVLGVIPKALMPTEISGASVGEVKIVSDMHERKAEMARQSDAFIALPGGYGTMEELLEMITWSQLGIHDKPVGLLNVDGYYDPLLALFDKGAAEGFIKADCRQIIVSAPTAHELLRKMEQYTRSHQEVAPRTSWEMSELGYGKTPEES